(19) United States
(12) Patent Application Publication
(10) Pub. No.: US 2026/0155258 A1
(43) Pub. Date: Jun. 4, 2026

(54) METHODS FOR DETECTING AGGRESSIVE PROSTATE CANCER

(71) Applicant: Minomic International Ltd., New South Wales (AU)

(72) Inventors: Douglas H Campbell, New South Wales (AU); Bradley J Walsh, New South Wales (AU); Yanling Lu, New South Wales (AU); Niantao Deng, New South Wales (AU)

(73) Assignee: Minomic International Ltd., New South Wales (AU)

(21) Appl. No.: 19/122,229

(22) PCT Filed: Oct. 20, 2023
(86) PCT No.: PCT/AU2023/051050

§ 371 Date: Apr. 17, 2025

(30) Foreign Application Priority Data
Oct. 20, 2022 (AU) 2022903101

(51) Int. Cl. G16H 50/30; G01N 33/575; G01N 33/57555; G16H 10/40; G16H 10/60

(52) U.S. Cl. CPC G16H 50/30; G01N 33/57555; G01N 33/57575; G16H 10/40; G16H 10/60

(57) ABSTRACT

The present invention provides methods for the detection of aggressive prostate cancer, and methods for detecting aggressive prostate cancer based on comparisons to a mixed control population of subjects with non-aggressive prostate cancer or not having prostate cancer.

### RELATED APPLICATIONS

This application claims priority to Australian provisional application AU2022903101, filed on 20 Oct. 2022. The contents of this application are incorporated in its entirety by reference herein.

### TECHNICAL FIELD

The present invention relates generally to the fields of immunology and medicine. More specifically, the present invention relates to the detection of aggressive of prostate cancer in subjects by assessing various combinations of biomarker/s and clinical variable/s.

### BACKGROUND

Prostate cancer is the most frequently diagnosed visceral cancer and the second leading cause of cancer death in males. According to the National Cancer Institute's SEER program and the Centers for Disease Control's National Center for Health Statistics, 164,690 cases of prostate cancer are estimated to have arisen in 2018 (9.5% of all new cancer cases) with an estimated 29,430 deaths (4.8% of all cancer deaths) (see SEER Cancer Statistics Factsheets: Prostate Cancer. National Cancer Institute. Bethesda, MD, http://seer.cancer.gov/statfacts/html/prost.html). The relative proportion of aggressive prostate cancers (defined as Gleason 3+4 or higher) to non-aggressive prostate cancers (defined as Gleason 3+3 or lower) differs between studies. A recent study of 1012 US men proceeding to prostate biopsy with elevated PSA demonstrated 542 men were negative for prostate cancer on biopsy, 239 had Gleason 3+3 prostate cancer and 231 had Gleason 3+4 or higher prostate cancer (Parekh et al. Eur. Urol. 2015 September; 68(3):464-70).

Commonly used screening tests for prostate cancer include digital rectal exam (DRE) and detection of prostate specific antigen (PSA) in blood. DRE is invasive and imprecise, and the prevalence of false negative (i.e. cancer undetected) and false positive (i.e. indication of cancer where none exists) results from PSA assays is well documented. The 2018 update to the AUA guideline for early detection of prostate cancer states that “Although DRE has been considered a mainstay of screening together with PSA, the Panel could find no evidence to support the continued use of DRE as a first line screening test.” (Carter H B, Albertsen P C, Barry M J et al: Early detection of prostate cancer: AUA Guideline. J Urol. 2013; 190: 419). DRE is being used less frequently in many countries due to patient preference and an increased use of multiparametric MRI.

In 2012, the United States Preventative Services Taskforce (USPTF) issued a recommendation against routine prostate cancer screening using the PSA test. This led to a decrease in the number of men proceeding to biopsy following elevated PSA test results and an increase in the proportion of men presenting with aggressive prostate cancer (Fleshner & Carlsson, Nature Reviews Urology, volume 15, pages 532-534, 2018).

Multiparametric Magnetic Resonance Imaging (mpMRI) is widely used in many countries following an elevated PSA. mpMRI enables visualisation of the prostate and grades the images using PIRADs or Likert scales that range from 1 (very unlikely that clinically significant prostate cancer is present) to 5 (highly likely that clinically significant prostate cancer is present). Patients with mpMRI scores of 4 and 5 will typically proceed to prostate biopsy, while those with scores of 1 and 2 will not. Biopsy decisions with patients with mpMRI scores of 3 are particularly challenging, with clinically significant cancer rates of as low as 12%, compared to 60% for PIRADS 4 and 83% PIRADs 5 (Kasivisvanathan et al 2018, PRECISION Study Group Collaborators. MRI-Targeted or Standard Biopsy for Prostate-Cancer Diagnosis. N Engl J Med. 2018; 378(19):1767). Despite current practice not recommending biopsy of patients with mpMRI scores of 1 and 2, up to 18% of such patients can harbour clinically significant prostate cancer (Doan et al, Eur. Urol. 2021 August; 80(2):260-261).

Upon a positive indication of prostate cancer due to PSA, DRE or mpMRI, confirmatory diagnostic tests include transrectal ultrasound, transrectal ultrasound guided biopsy, transperineal biopsy and MRI guided biopsies. However, these techniques are invasive and cause significant discomfort to the subject under examination.

A general need exists for more convenient, reliable and/or accurate tests capable of detecting aggressive prostate cancer.

### SUMMARY OF THE INVENTION

The present inventors have identified combinations of biomarker/s and clinical variable/s effective for detecting aggressive prostate cancer. Accordingly, the biomarker/clinical variable combinations disclosed herein can be used to detect the presence or absence of aggressive prostate cancer in a subject. In some cases, detecting aggressive prostate cancer using the biomarker/clinical variable combinations may reduce dependence on DRE and leverage information available from the mpMRI diagnostic pathway, with an emphasis, for example, on patients with mpMRI scores of 1-3.

The present invention relates at least to the following series of numbered embodiments below:

Embodiment 1. A method for detecting aggressive prostate cancer (CaP) in a test subject, comprising:
(a) having obtained an analyte level for one or more analytes in the test subject's biological sample, and having obtained a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject is likely to have aggressive CaP by comparison of the subject test score value and the threshold value,
wherein:
the one or more analyte/s comprise or consist of WAP four-disulfide core domain protein 2 (WFDC2 (HE4)), and optionally total prostate surface antigen (PSA),
the one or more clinical variables comprise at least one of: % Free PSA, Free PSA, DRE, Prostate Volume (PV), PIRADs score, Age, Family History (FH) and
the threshold value was determined by:
measuring said one or more analyte/s in a series of biological samples obtained from a population of subjects having aggressive CaP and from a population of control subjects not having aggressive CaP, to thereby obtain an analyte level for each said analyte in each said biological sample of the series;
combining each said analyte level of the series with measurements of said one or more clinical variables obtained from each said subject of the populations, in a manner that allows discrimination between aggressive CaP and an absence of aggressive CaP, to thereby generate the threshold value.

Embodiment 2. The method of embodiment 1, wherein the population of control subjects comprises or consists of subjects that do not have prostate cancer.

Embodiment 3. The method of embodiment 1, wherein the population of control subjects comprises or consists of subjects that do not have aggressive prostate cancer.

Embodiment 4. The method of embodiment 1 or embodiment 3, wherein the population of control subjects has non-aggressive CaP as defined by a Gleason score of 3+3 or do not have prostate cancer.

Embodiment 5. The method of any one of embodiments 1 to 4, wherein the threshold value is determined prior to performing the method.

Embodiment 6. The method of any one of embodiments 1 to 5, wherein the one or more clinical variables and the one or more analyte/s comprise or consist of any one of the following:
WFDC2 (HE4), total PSA, % Free PSA and Age
WFDC2 (HE4), total PSA, % Free PSA, PV and Age
WFDC2 (HE4), total PSA, % Free PSA and PIRADs
WFDC2 (HE4), total PSA, % Free PSA, PV and PIRADs
WFDC2 (HE4), total PSA, % Free PSA, PV, PIRADs and Age
WFDC2 (HE4), total PSA, % Free PSA, PV, PIRADs, Age and DRE
WFDC2 (HE4), total PSA, % Free PSA, Age and DRE
WFDC2 (HE4), total PSA, % Free PSA, Age and FH
WFDC2 (HE4), total PSA, % Free PSA, Age, DRE, FH
WFDC2 (HE4), % Free PSA and Age
WFDC2 (HE4), % Free PSA and PIRADs
WFDC2 (HE4), % Free PSA, PV and Age
WFDC2 (HE4), % Free PSA, PV and PIRADs
WFDC2 (HE4), % Free PSA, PV, Age and PIRADs
WFDC2 (HE4), % Free PSA, PV, Age, PIRADS and DRE
WFDC2 (HE4), total PSA, Free PSA
WFDC2 (HE4), total PSA, Free PSA and PV
WFDC2 (HE4), total PSA, Free PSA, PV and Age
WFDC2 (HE4), total PSA, Free PSA, PV and PIRADs
WFDC2 (HE4), total PSA, Free PSA, PV, PIRADs and Age
WFDC2 (HE4), total PSA, Free PSA and Age
WFDC2 (HE4), total PSA, Free PSA and PIRADs

Embodiment 7. The method of any one of embodiments 1 to 6, comprising selecting a subset of the combined analyte/s and/or clinical variable measurements to generate the threshold value.

Embodiment 8. The method of any one of embodiments 1 to 7, wherein said combining of each said analyte level of the series with said measurements of the one or more clinical variables comprises combining a logistic regression score of the clinical variable measurements and analyte level/s in a manner that maximizes said discrimination, in accordance with the formula:

$$\begin{matrix}Logit ( P ) = Log ( P / 1 - P ) \\ = intercept + {\sum }_{i = 1}^{N} ( {coefficient}_{i} \times transformed ( {variable}_{i} ) \\ P = \frac{exp ( Logit ( P ) )}{1 + exp ( Logit ( P ) )}\end{matrix} \qquad ( i )$$

wherein:
P is probability of that the test subject has aggressive prostate cancer,
the coefficientᵢ is the natural log of the odds ratio of the variable,
the transformed variableᵢ is the natural log of the variableᵢ value; or

$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( ii )$$

wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable,
the variableⱼ is the numerical value of the variableⱼ; or

$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( iii )$$

wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable,
the variableⱼ is the numerical value of the variableⱼ,
variableⱼ can be one or more of DRE value, Age, or PIRADS score,
if variableⱼ is DRE, a DRE value of 1 indicates an abnormal DRE status and a DRE value of 0 indicates a normal DRE status,
if variableⱼ is PIRADs score, the PIRADS score is 1, 2, 3, 4 or 5, and if a PIRADS score is not available, variableⱼ is 0, and
if variableⱼ is Age, the Age is in years.

Embodiment 9. The method of any one of embodiments 1 to 8, wherein said applying a suitable algorithm and/or transformation to the combination of the clinical variable measurements and analyte level/s comprises use of an exponential function, a logarithmic function, a power function and/or a root function.

Embodiment 10. The method according to any one of embodiments 1 to 9, wherein the suitable algorithm and/or transformation applied to the combination of the clinical variable measurements and analyte level/s of the test subject is in accordance with the formula:

$$\begin{matrix}Logit ( P ) = Log ( P / 1 - P ) \\ = intercept + {\sum }_{i = 1}^{N} ( {coefficient}_{i} \times transformed ( {variable}_{i} ) \\ P = \frac{exp ( Logit ( P ) )}{1 + exp ( Logit ( P ) )}\end{matrix} \qquad ( i )$$

wherein:
P is probability of that the test subject has aggressive prostate cancer,
the coefficientᵢ is the natural log of the odds ratio of the variable,
the transformed variableᵢ is the natural log of the variableᵢ value; or

$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( ii )$$

wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable,
the variableⱼ is the numerical value of the variableⱼ; or

$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( iii )$$

wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable,
the variableⱼ is the numerical value of the variableⱼ,
variableⱼ can be one or more of DRE value, Age, or PIRADS score,
if variableⱼ is DRE, a DRE value of 1 indicates an abnormal DRE status and a DRE value of 0 indicates a normal DRE status,
if variableⱼ is PIRADs score, the PIRADS score is 1, 2, 3, 4 or 5, and if a PIRADS score
is not available, variableⱼ is 0, and
if variableⱼ is Age, the Age is in years.

Embodiment 11. The method according to any one of embodiments 1 to 10, wherein said combining of each said analyte level of the series with measurements of said one or more clinical variables obtained from each said subject of the populations maximizes said discrimination.

Embodiment 12. The method of any one of embodiments 1 to 11, wherein said combining of each said analyte level of the series with the measurements of one or more clinical variables obtained from each said subject of the populations is conducted in a manner that:
(i) reduces the misclassification rate between the subjects likely to have aggressive CaP and said control subjects; and/or
(ii) increases sensitivity in discriminating between the subjects likely to have aggressive CaP and said control subjects; and/or
(iii) increases specificity in discriminating between the subjects likely to have aggressive CaP and said control subjects.

Embodiment 13. The method of embodiment 12, wherein said combining in a manner that reduces the misclassification rate between the subjects likely to have aggressive CaP and said control subjects comprises selecting a suitable true positive and/or true negative rate.

Embodiment 14. The method of embodiment 12, wherein said combining in a manner that reduces the misclassification rate between the subjects likely to have aggressive CaP and said control subjects minimizes the misclassification rate.

Embodiment 15. The method of embodiment 12, wherein said combining in a manner that reduces the misclassification rate between the subjects likely to have aggressive CaP and said control subjects comprises minimizing the misclassification rate between the subjects likely to have aggressive CaP and said control subjects by identifying a point where the true positive rate intersects the true negative rate.

Embodiment 16. The method of embodiment 12, wherein said selecting the threshold value from the combined clinical variable measurement/s and combined analyte level/s in a manner that increases sensitivity in discriminating between the subjects likely to have aggressive CaP and said control subjects increases or maximizes said sensitivity.

Embodiment 17. The method of embodiment 12, wherein said selecting the threshold value from the combined clinical variable measurement/s and combined analyte level/s in a manner that increases specificity in discriminating between the subjects likely to have aggressive CaP and said control subjects increases or maximizes said specificity.

Embodiment 18. The method according to any one of embodiments 1 to 17, wherein the one or more clinical variables and the one or more analytes comprise or consist of:
total PSA, % free PSA, WFDC2 (HE4), Age or
total PSA, % free PSA, WFDC2 (HE4), Age, PV.

Embodiment 19. The method according to any one of embodiments 1 to 18, wherein the test subject has previously received a positive indication of prostate cancer.

Embodiment 20. The method according to any one of embodiments 1 to 19, wherein the test subject has previously received a positive indication of prostate cancer by digital rectal exam (DRE) and/or by PSA testing.

Embodiment 21. The method according to any one of embodiments 1 to 19, wherein the test subject has previously received a positive indication of prostate cancer by MRI PIRADs score.

Embodiment 22. The method according to any one of embodiments 1 to 19, wherein the test subject has previously received a positive indication of prostate cancer by DRE and/or PSA testing with MRI PIRADs score of 1-3.

Embodiment 23. The method according to any one of embodiments 1 to 19, wherein the test subject has previously received a positive indication of prostate cancer by DRE and/or PSA testing with MRI PIRADs score of 3.

Embodiment 24. The method according to any one of embodiments 1 to 23, wherein the series of biological samples obtained from each said population and/or the test subject's biological sample are selected from: whole blood, serum, plasma, saliva, tear/s, urine, tissue, and any combination thereof.

Embodiment 25. The method according to any one of embodiments 1 to 24, wherein said test subject, said population of subjects likely to have aggressive CaP, and said population of control subjects are human.

Embodiment 26. The method of any one of embodiments 1 to 25, further comprising measuring one or more analyte/s in the test subject's biological sample to thereby obtain the analyte level for each said one or more analytes.

Embodiment 27. The method according to embodiment 26, wherein said measuring of one or more analyte/s in the test subject's biological sample comprises:
(i) measuring one or more fluorescent signals indicative of each said analyte level;
(ii) obtaining a measurement of weight/volume or molarity of said analyte/s in the biological sample;
(iii) measuring an absorbance signal indicative of each said analyte level; or
(iv) using a technique selected from the group consisting of: electrochemiluminescence, mass spectrometry, a protein array technique, high performance liquid chromatography (HPLC), gel electrophoresis, radiolabeling, and any combination thereof.

Embodiment 28. The method according to embodiment 26 or embodiment 27, wherein the test subject's biological sample is contacted, or the series of biological samples was contacted, with first and second antibody populations for detection of each said analyte, wherein each said antibody population has binding specificity for one of said analytes, and the first and second antibody populations have different analyte binding specificities.

Embodiment 29. The method according to embodiment 28, wherein the first and/or second antibody populations are labelled.

Embodiment 30. The method according to embodiment 29, wherein the first and/or second antibody populations comprise a label selected from the group consisting of a radiolabel, a fluorescent label, a biotin-avidin amplification system, a chemiluminescence system, microspheres, and colloidal gold.

Embodiment 31. The method according to embodiment 28 or 29, wherein binding of each said antibody population to the analyte is detected by a technique selected from the group consisting of: immunofluorescence, radiolabeling, immunoblotting, Western blotting, enzyme-linked immunosorbent assay (ELISA), flow cytometry, immunoprecipitation, immunohistochemistry, biofilm test, affinity ring test, antibody array, optical density test, and chemiluminescence.

Embodiment 32. The method of any one of embodiments 26 to 31, wherein said measuring of each said analyte in the biological sample from the test subject or the series of biological samples obtained from each said population comprises measuring the analytes directly.

Embodiment 33. The method of any one of embodiments 26 to 31, wherein said measuring of each said analyte in the biological sample from the test subject or the series of biological samples obtained from each said population comprises detecting a nucleic acid encoding the analytes.

Embodiment 34. The method of any one of embodiments 1 to 33, further comprising measuring the two one or more clinical variables in the test subject.

Embodiment 35. The method of any one of embodiments 1 to 34, further comprising determining said threshold value.

Embodiment 36. The method of embodiment 35, wherein determining said threshold value comprises measuring said one or more analyte/s in a series of biological samples obtained from a population of subjects having aggressive CaP and from a population of control subjects not having aggressive CaP, to thereby obtain an analyte level for each said analyte in each said biological sample of the series.

Embodiment 37. The method of any one of embodiments 1 to 36, further comprising a step of obtaining a biopsy from the test subject to confirm whether the test subject has aggressive CaP.

Embodiment 38. The method of embodiment 37, further comprising a step of treating a test subject confirmed to have aggressive CaP, optionally wherein the treatment is selected from one or more of surgery (e.g., radical prostatectomy), chemotherapy, radiation therapy (e.g., external beam radiation therapy or brachytherapy (with or without concomitant androgen deprivation therapy)), immunotherapy, hormone therapy, drug treatment and combinations thereof.

Embodiment 39. A method of treating aggressive prostate cancer in a test subject comprising:
(a) having obtained an analyte level for one or more analytes in the test subject's biological sample, and having obtained a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject is likely to have aggressive CaP by comparison of the subject test score value and the threshold value,
wherein:
the one or more analyte/s comprise or consist of WAP four-disulfide core domain protein 2 (WFDC2 (HE4)), and optionally total prostate surface antigen (PSA),
the one or more clinical variables comprise at least one of: % Free PSA, Free PSA, DRE, Prostate Volume (PV), PIRADs score, Age, Family History (FH) and the threshold value was determined by:
measuring said one or more analyte/s in a series of biological samples obtained from a population of subjects having aggressive CaP and from a population of control subjects not having aggressive CaP, to thereby obtain an analyte level for each said analyte in each said biological sample of the series;
combining each said analyte level of the series with measurements of said one or more clinical variables obtained from each said subject of the populations, in a manner that allows discrimination between aggressive CaP and an absence of aggressive CaP, to thereby generate the threshold value,
wherein the method further comprises:
(i) obtaining a biopsy from the test subject to confirm the presence of aggressive CaP; and
(ii) treating the test subject confirmed to have aggressive CaP, preferably with one or more of surgery (e.g., radical prostatectomy), chemotherapy, radiation therapy (e.g., external beam radiation therapy or brachytherapy (with or without concomitant androgen deprivation therapy)), immunotherapy, hormone therapy or drug treatment or combinations thereof.

Embodiment 40. A surgery (e.g., radical prostatectomy), chemotherapy, radiation therapy (e.g., external beam radiation therapy or brachytherapy (with or without concomitant androgen deprivation therapy)), immunotherapy, hormone therapy or drug for use in the treatment of aggressive prostate cancer in a test subject, the treatment comprising:
(a) having obtained an analyte level for one or more analytes in the test subject's biological sample, and having obtained a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject is likely to have aggressive CaP by comparison of the subject test score value and the threshold value,
wherein:
the one or more analyte/s comprise or consist of WAP four-disulfide core domain protein 2 (WFDC2 (HE4)), and optionally total prostate surface antigen (PSA),
the one or more clinical variables comprise at least one of: % Free PSA, Free PSA, DRE, Prostate Volume (PV), PIRADs score, Age, Family History (FH) and
the threshold value was determined by:
measuring said one or more analyte/s in a series of biological samples obtained from a population of subjects having aggressive CaP and from a population of control subjects not having aggressive CaP, to thereby obtain an analyte level for each said analyte in each said biological sample of the series;
combining each said analyte level of the series with measurements of said one or more clinical variables obtained from each said subject of the populations, in a manner that allows discrimination between aggressive CaP and an absence of aggressive CaP, to thereby generate the threshold value,
wherein the method further comprises:
(iii) obtaining a biopsy from the test subject to confirm the presence of aggressive CaP; and
(iv) treating the test subject confirmed to have aggressive CaP with one or more of the surgery (e.g., radical prostatectomy), chemotherapy, radiation therapy (e.g., external beam radiation therapy or brachytherapy (with or without concomitant androgen deprivation therapy)), immunotherapy, hormone therapy, drug treatment or combinations thereof.

Embodiment 41. Use of chemotherapy, radiation therapy (e.g., external beam radiation therapy or brachytherapy (with or without concomitant androgen deprivation therapy)), immunotherapy, hormone therapy or drug treatment in the manufacture of a medicament for the treatment of aggressive prostate cancer in a test subject, the treatment comprising:
(a) having obtained an analyte level for one or more analytes in the test subject's biological sample, and having obtained a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject is likely to have aggressive CaP by comparison of the subject test score value and the threshold value,
wherein:
the one or more analyte/s comprise or consist of WAP four-disulfide core domain protein 2 (WFDC2 (HE4)), and optionally total prostate surface antigen (PSA), the one or more clinical variables comprise at least one of: % Free PSA, Free PSA, DRE, Prostate Volume (PV), PIRADs score, Age, Family History (FH) and
the threshold value was determined by:
measuring said one or more analyte/s in a series of biological samples obtained from a population of subjects having aggressive CaP and from a population of control subjects not having aggressive CaP, to thereby obtain an analyte level for each said analyte in each said biological sample of the series;
combining each said analyte level of the series with measurements of said one or more clinical variables obtained from each said subject of the populations, in a manner that allows discrimination between aggressive CaP and an absence of aggressive CaP, to thereby generate the threshold value,
wherein the method further comprises:
(v) obtaining a biopsy from the test subject to confirm the presence of aggressive CaP; and
(vi) treating the test subject confirmed to have aggressive CaP with one or more of the chemotherapy, radiation therapy (e.g., external beam radiation therapy or brachytherapy (with or without concomitant androgen deprivation therapy)), immunotherapy, hormone therapy or drug treatment.

Embodiment 42. The method of embodiment 39, the surgery (e.g., radical prostatectomy), chemotherapy, radiation therapy (e.g., external beam radiation therapy or brachytherapy (with or without concomitant androgen deprivation therapy)), immunotherapy, hormone therapy or drug for use of embodiment 40 or the use of embodiment 41, further comprising one or more of the features or steps defined in any one of embodiments 2 to 36.

Embodiment 43. A kit for use in the method according to any one of embodiments 1 to 36, comprising reagents and/or components for determining the clinical variable/s and the one or more biomarker/s defined in any one of embodiments 1 to 36.

It will be understood that the invention disclosed and defined in this specification extends to all alternative combinations of two or more of the individual features or embodiments above or in the specification. All of these different combinations constitute various alternative aspects of the invention.

### BRIEF DESCRIPTION OF THE FIGURES

Preferred embodiments of the present invention will now be described, by way of example only, with reference to the accompanying figures wherein:

Figure One. PSA in MQ192—Depicts a ROC curve analysis based on PSA levels in the MQ population (model 1, fitting: logistic regression) generated to differentiate [aggressive prostate cancer (AgCaP) versus patients without-aggressive prostate cancer (NotAgCaP)].

Figure Two. PV in MQ 192—Depicts a ROC curve analysis based on Prostate Volume in the MQ population (model 2, fitting: logistic regression) generated to differentiate [aggressive prostate cancer (AgCaP) versus patients without-aggressive prostate cancer (NotAgCaP)].

Figure Three. Percent free PSA in MQ—Depicts a ROC curve analysis based on % free PSA in the MQ population (model 3, fitting: logistic regression) generated to differentiate [aggressive prostate cancer (AgCaP) versus patients without-aggressive prostate cancer (NotAgCaP)].

Figure Four. Free PSA in MQ—Depicts a ROC curve analysis based on free PSA in the MQ population (model 4, fitting: logistic regression) generated to differentiate [aggressive prostate cancer (AgCaP) versus patients without-aggressive prostate cancer (NotAgCaP)].

Figure Five. HE4 in MQ—Depicts a ROC curve analysis based on WFDC2(HE4) in the MQ population (model 5, fitting: logistic regression) generated to differentiate [aggressive prostate cancer (AgCaP) versus patients without-aggressive prostate cancer (NotAgCaP)].

Figure Six. PIRADs in MQ—Depicts a ROC curve analysis based on PIRADs in the MQ population (model 6, fitting: logistic regression) generated to differentiate [aggressive prostate cancer (AgCaP) versus patients without-aggressive prostate cancer (NotAgCaP)].

Figure Seven. Age in MQ 192—Depicts a ROC curve analysis based on Age (model 7, fitting: logistic regression) generated to differentiate [aggressive prostate cancer (AgCaP) versus patients without-aggressive prostate cancer (NotAgCaP)].

Figure Eight. DRE in MQ 192—Depicts a ROC curve analysis based on DRE (model 8, fitting: logistic regression) generated to differentiate [aggressive prostate cancer (AgCaP) versus patients without-aggressive prostate cancer (NotAgCaP)].

Figure Nine. HE4, PSA, % Free PSA in MQ192—Depicts a ROC curve analysis based on WFDC2(HE4), PSA, % free PSA (model 9 fitting: logistic regression) generated to differentiate [aggressive prostate cancer (AgCaP) versus patients without-aggressive prostate cancer (NotAgCaP)].

Figure Ten. HE4, PSA, % free PSA, Age in MQ 192—Depicts a ROC curve analysis based on WFDC2(HE4), PSA, % free PSA, Age (model 16 fitting: logistic regression) generated to differentiate [aggressive prostate cancer (AgCaP) versus patients without-aggressive prostate cancer (NotAgCaP)].

Figure Eleven. HE4, PSA, % free PSA, Age, PV MQ192—Depicts a ROC curve analysis based on WFDC2(HE4), PSA, % free PSA, Age, PV (model 11 fitting: logistic regression) generated to differentiate [aggressive prostate cancer (AgCaP) versus patients without-aggressive prostate cancer (NotAgCaP)].

Figure Twelve. PSA in MQ49—Depicts a ROC curve analysis based on PSA, (model 35, fitting: logistic regression) generated to differentiate [aggressive prostate cancer (AgCaP) versus patients without-aggressive prostate cancer (NotAgCaP)]. Developed on 49 patients.

Figure Thirteen. HE4, PSA, % free PSA, Age, PV in MQ49—Depicts a ROC curve analysis based on WFDC2(HE4), PSA, % free PSA, Age, PV (model 38, fitting: logistic regression) generated to differentiate [aggressive prostate cancer (AgCaP) versus patients without-aggressive prostate cancer (NotAgCaP)]. Developed on 49 patients.

Figure Fourteen. PSA in CUSP 302 on Abbott Architect—Depicts a ROC curve analysis based on PSA (model 42 fitting: logistic regression) generated to differentiate [aggressive prostate cancer (AgCaP) versus patients without-aggressive prostate cancer (NotAgCaP)] in the US population using the Abbott analyzer.

Figure Fifteen. WFDC2(HE4), PSA, % free PSA in CUSP 302—Depicts a ROC curve analysis based on WFDC2(HE4), PSA, % free PSA, (model 44, fitting: logistic regression) generated to differentiate [aggressive prostate cancer (AgCaP) versus patients without-aggressive prostate cancer (NotAgCaP)] in the US population using the Abbott Analyzer.

Figure Sixteen. WFDC2(HE4), PSA, % free PSA, Age, PV, in US 302 on Abbott—Depicts a ROC curve analysis based on WFDC2(HE4), PSA, % free PSA, Age, PV (model 46, fitting: logistic regression) generated to differentiate [aggressive prostate cancer (AgCaP) versus patients without-aggressive prostate cancer (NotAgCaP)] in the US population using the Abbott analyzer.

Figure Seventeen. PSA, in US on Roche 300—Depicts a ROC curve analysis based on PSA (model 43, fitting: logistic regression) generated to differentiate [aggressive prostate cancer (AgCaP) versus patients without-aggressive prostate cancer (NotAgCaP)] in the US population using the Roche analyzer.

Figure Eighteen. HE4, PSA, % free PSA, in US on Roche 300—Depicts a ROC curve analysis based on WFDC2(HE4), PSA, % free PSA (model 48, fitting: logistic regression) generated to differentiate [aggressive prostate cancer (AgCaP) versus patients without-aggressive prostate cancer (NotAgCaP)] in the US population using the Roche analyzer.

Figure Nineteen. WFDC2(HE4), PSA, % free PSA, Age, PV in US on Roche—Depicts a ROC curve analysis based on WFDC2(HE4), PSA, % free PSA, Age, PV (model 50, fitting: logistic regression) generated to differentiate [aggressive prostate cancer (AgCaP) versus patients without-aggressive prostate cancer (NotAgCaP)] in the US population using the Roche analyzer.

Figure Twenty. WFDC2(HE4), PSA, % free PSA, Age, PV CV model in MQ 192—Depicts a ROC curve analysis based on WFDC2(HE4), PSA, % free PSA, Age, PV (model 71, fitting: cross-validated logistic regression) generated to differentiate [aggressive prostate cancer (AgCaP) versus patients without-aggressive prostate cancer (NotAgCaP)] developed on the 192 MQ population and applied to the 192 MQ population.

Figure Twenty One. HE4, PSA, % free PSA, Age, PV CV 192 on 302 CUSP—Depicts a ROC curve analysis based on WFDC2(HE4), PSA, % free PSA, Age, PV (model 72, fitting: cross-validated logistic regression) generated to differentiate [aggressive prostate cancer (AgCaP) versus patients without-aggressive prostate cancer (NotAgCaP)] developed on the 192 MQ population and applied to the 302 CUSP population.

Figure Twenty two. HE4, PSA, % free PSA, Age, PV CV 192 on 49—Depicts a ROC curve analysis based on WFDC2(HE4), PSA, % free PSA, Age, PV (model 73, fitting: cross-validated logistic regression) generated to differentiate [aggressive prostate cancer (AgCaP) versus patients without-aggressive prostate cancer (NotAgCaP)] developed on the 192 MQ population and applied to the 49 PIRADs 1-3 population. The equivalent ROC curve for PSA in this population is also shown (model 35).

Figure Twenty Three. WFDC2(HE4), PSA, % free PSA, Age CV model in 506—Depicts a ROC curve analysis based on WFDC2(HE4), PSA, % free PSA, Age (model 77, fitting: cross-validated logistic regression) generated to differentiate [aggressive prostate cancer (AgCaP) versus patients without-aggressive prostate cancer (NotAgCaP)] developed on the 506 (192 MQ+314 CUSP populations) and applied to the 506 population.

Figure Twenty Four. HE4, PSA, % free PSA, Age CV 506 on 192—Depicts a ROC curve analysis based on WFDC2(HE4), PSA, % free PSA, Age (model 78, fitting: cross-validated logistic regression) generated to differentiate [aggressive prostate cancer (AgCaP) versus patients without-aggressive prostate cancer (NotAgCaP)] developed on the 506 (192 MQ+314 CUSP populations) and applied to the 192 MQ population.

Figure Twenty Five. HE4, PSA, % free PSA, Age CV 506 on 314—Depicts a ROC curve analysis based on WFDC2(HE4), PSA, % free PSA, Age (model 79, fitting: cross-validated logistic regression) generated to differentiate [aggressive prostate cancer (AgCaP) versus patients without-aggressive prostate cancer (NotAgCaP)] developed on the 506 (192 MQ+314 CUSP populations) and applied to the 314 CUSP population.

Figure Twenty Six. Biopsy reductions with MiCheck post-MRI—Shows the reduction in biopsies for no CaP, non-aggressive CaP and Aggressive CaP groups if MiCheck® Prostate were used to guide a biopsy decision in the post-MRI setting.

Figure Twenty Seven. Biopsy reductions with MiCheck pre-MRI—Shows the reduction in biopsies for no CaP, non-aggressive CaP and Aggressive CaP groups if MiCheck® Prostate were used to guide a biopsy decision in the pre-MRI setting.

Figure Twenty Eight. ROC Curve Comparison of PSA (Model 1) and MiCheck® Prostate MRI (Model 73) on MQ192 population—Depicts a ROC curve comparison of PSA (Model 1) vs. Model 73 [WFDC2(HE4), PSA, % free PSA, Age, Prostate Volume, fitting: cross-validated logistic regression] applied to the MQ 192 population.

Figure Twenty Nine. ROC Curve Comparison of PSA (Model 1) and MiCheck® Prostate non-MRI (Model 79) on MQ192 population—Depicts a ROC curve comparison of PSA (Model 1) vs. Model 79 [WFDC2(HE4), PSA, % free PSA, Age, fitting: cross-validated logistic regression] applied to the MQ 192 population.

Figure Thirty. ROC Curve Comparison of MiCheck® Prostate non-MRI (Model 79) and PIRADS (Model 6) on MQ192 population—Depicts a ROC curve comparison of MiCheck® Prostate MRI Model 79 [WFDC2(HE4), PSA, % free PSA, Age, fitting: cross-validated logistic regression] and PIRADs (model 6) applied to the MQ 192 population.

### DEFINITIONS

As used in this application, the singular form “a”, “an” and “the” include plural references unless the context clearly dictates otherwise. For example, the phrase “an antibody” also includes multiple antibodies.

As used herein, the term “comprising” means “including.” Variations of the word “comprising”, such as “comprise” and “comprises,” have correspondingly varied meanings. The term is not intended to be construed as exclusive unless the context suggests otherwise.

As used herein, the terms “aggressive prostate cancer” and “aggressive CaP” refer to prostate cancer with a primary Gleason score of 3 or greater and a secondary Gleason score of 4 or greater (GS≥3+4).

As used herein, the terms “non-aggressive prostate cancer” and “non-aggressive CaP” refer to prostate cancer with a primary Gleason score of less than or equal to 3 and a secondary Gleason score of less than 4 (GS≤3+3). Primary Gleason scores of less than 3 were not reported in the subject sample sets described in this application hence the term GS3+3 is also used for non-aggressive prostate cancer.

As used herein, the terms “WFDC2” and “HE4” will be understood to refer to the same analyte (WAP Four-disulfide core domain protein 2), and can be used together or interchangeably (e.g. WFDC2 (HE4)). A non-limiting example of an WFDC2/HE4 protein is provided under UniProtKB—Q14508 (see https://www.uniprot.org/uniprot/Q14508).

As used herein, the term “clinical variable” encompasses any factor, measurement, physical characteristic relevant in assessing prostate disease, including but not limited to: age, prostate volume (PV), % free PSA, free PSA, PSA velocity, PSA density, digital rectal examination (DRE), Age, ethnic background, family history (FH) of prostate cancer, a prior negative biopsy for prostate cancer or PIRADs score derived from MRI.

As used herein, the term “total PSA” and “Central PSA” are used interchangeably and have the same meaning, referring to a test capable of measuring free plus complexed PSA in a sample.

As used herein, the term “% free PSA” refers to the ratio of free/total PSA in a sample expressed as a percentage.

As used herein, the term “free PSA” refers to PSA that is not attached to other blood proteins.

As used herein, the term “PSA level” refers to nanograms of PSA per milliliter (ng/mL) of blood in a test patient.

As used herein, the term “Free PSA level” refers to nanograms of PSA per milliliter (ng/mL) of blood in a test patient.

As used herein, the term “WFDC2(HE4) level” refers to picomoles of HE4 per milliliter (pmol/mL) of blood in a test patient.

As used herein, the terms “MRI PIRADS” and “PIRADS” are used interchangeably and will be understood to have the same meaning, being a structured reporting scheme for multiparametric prostate MRI in the evaluation of suspected prostate cancer in treatment naive prostate glands.

As used herein, the terms “biological sample” and “sample” encompass any body fluid or tissue taken from a subject including, but not limited to, a saliva sample, a tear sample, a blood sample, a serum sample, a plasma sample, a urine sample, or sub-fractions thereof.

As used herein, the terms “Family History” and its abbreviation “FH” will be understood to mean a determination of whether a family history of prostate cancer exists on either side of the family of given subject including, for example, those with a first-degree relative who was diagnosed at age <65 years.

As used herein, the terms “diagnosing” and “diagnosis” refer to methods by which a person of ordinary skill in the art can estimate and even determine whether or not a subject is suffering from a given disease or condition. A diagnosis may be made, for example, on the basis of one or more diagnostic indicators, such as for example, the detection of a combination of biomarker/s and clinical feature/s as described herein, the levels of which are indicative of the presence, severity, or absence of the condition. As such, the terms “diagnosing” and “diagnosis” thus also include identifying a risk of developing aggressive prostate cancer.

The terms “treatment” or “treating” a subject includes the application or administration of an agent, drug or compound to a subject with the purpose of delaying, slowing, stabilizing, curing, healing, alleviating, relieving, altering, remedying, less worsening, ameliorating, improving, or affecting the disease or condition, the symptom of the disease or condition, or the risk of the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

Suitable treatments as identified by a skilled person are to be administered in an effective amount. The phrase ‘therapeutically effective amount’ or ‘effective amount’ generally refers to an amount of a suitable treatment that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g., side effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

As used herein, the terms “subject” and “patient” are used interchangeably unless otherwise indicated, and encompass any animal of economic, social or research importance including bovine, equine, ovine, primate, avian and rodent species. Hence, a “subject” may be a mammal such as, for example, a human or a non-human mammal. As used herein, the term “isolated,” “recombinant” or “synthetic” in reference to a biological molecule (e.g. an antibody) is a biological molecule that is free from at least some of the components with which it naturally occurs.

As used herein, the terms “antibody” and “antibodies” include IgG (including IgG1, IgG2, IgG3, and IgG4), IgA (including IgAQ1 and IgA2), IgD, IgE, IgM, and IgY, whole antibodies, including single-chain whole antibodies, and antigen-binding fragments thereof. Antigen-binding antibody fragments include, but are not limited to, Fv, Fab, Fab′ and F(ab′)2, Fd, single-chain Fvs (scFv), single-chain antibodies, disulfide-linked Fvs (sdFv) and fragments comprising either a VL or VH domain. The antibodies may be from any animal origin or appropriate production host. Antigen-binding antibody fragments, including single-chain antibodies, may comprise the variable region/s alone or in combination with the entire or partial of the following: hinge region, CH1, CH2, and CH3 domains. Also included are any combinations of variable region/s and hinge region, CH1, CH2, and CH3 domains. Antibodies may be monoclonal, polyclonal, chimeric, multispecific, humanized, and human monoclonal and polyclonal antibodies which specifically bind the biological molecule. The antibody may be a bi-specific antibody, avibody, diabody, tribody, tetrabody, nanobody, single domain antibody, VHH domain, human antibody, fully humanized antibody, partially humanized antibody, anticalin, adnectin, or affibody.

As used herein, the terms “binding specifically” and “specifically binding” in reference to an antibody, antibody variant, antibody derivative, antigen binding fragment, and the like refers to its capacity to bind to a given target molecule preferentially over other non-target molecules. For example, if the antibody, antibody variant, antibody derivative, or antigen binding fragment (“molecule A”) is capable of “binding specifically” or “specifically binding” to a given target molecule (“molecule B”), molecule A has the capacity to discriminate between molecule B and any other number of potential alternative binding partners. Accordingly, when exposed to a plurality of different but equally accessible molecules as potential binding partners, molecule A will selectively bind to molecule B and other alternative potential binding partners will remain substantially unbound by molecule A. In general, molecule A will preferentially bind to molecule B at least 10-fold, preferably 50-fold, more preferably 100-fold, and most preferably greater than 100-fold more frequently than other potential binding partners. Molecule A may be capable of binding to molecules that are not molecule B at a weak, yet detectable level. This is commonly known as background binding and is readily discernible from molecule B-specific binding, for example, by use of an appropriate control.

As used herein, the term “kit” refers to any delivery system for delivering materials. Such delivery systems include systems that allow for the storage, transport, or delivery of reaction reagents (for example labels, reference samples, supporting material, etc. in the appropriate containers) and/or supporting materials (for example, buffers, written instructions for performing an assay etc.) from one location to another. For example, kits may include one or more enclosures, such as boxes, containing the relevant reaction reagents and/or supporting materials.

It will be understood that use of the term “between,” when referring to a range of numerical values, encompasses the numerical values at each endpoint of the range. For example, a polypeptide of between 10 residues and 20 residues in length is inclusive of a polypeptide of 10 residues in length and a polypeptide of 20 residues in length.

Any description of prior art documents herein, or statements herein derived from or based on those documents, is not an admission that the documents or derived statements are part of the common general knowledge of the relevant art. For the purposes of description all documents referred to herein are hereby incorporated by reference in their entirety unless otherwise stated.

### Abbreviations

As used herein the abbreviation “CaP” refers to prostate cancer.

As used herein the abbreviation “PSA” refers to prostate specific antigen.

As used herein the abbreviation “WFDC2” refers to WAP Four-disulfide core domain protein 2, also known in the art as Human Epididymis Protein 4 (HE4).

As used herein the abbreviation “Acc” refers to accuracy.

As used herein the abbreviation “Sens” refers to sensitivity.

As used herein the abbreviations “Spec” or “Specs” refers to specificity.

As used herein the abbreviation “AUC” refers to Area Under the ROC Curve

As used herein the abbreviation “ROC” refers to the Receiver Operator Characteristics Curve

As used herein the abbreviation “Log” refers to the natural logarithm.

As used herein the abbreviation “DRE” refers to digital rectal examination.

As used herein the abbreviation “NPV” refers to negative predictive value.

As used herein the abbreviation “PPV” refers to positive predictive value.

As used herein the abbreviation “AgCaP” refers to aggressive prostate cancer defined as prostate cancer with a Gleason score of 3+4 or greater.

As used herein the abbreviation “NonAgCaP” refers to non-aggressive prostate cancer defined as prostate cancer with a Gleason score of 3+3.

As used herein the abbreviation “NOT-AgCaP” refers to samples from subjects that do not have aggressive prostate cancer. These subjects may have non-aggressive prostate cancer or not have prostate cancer at all.

As used herein the abbreviation “mpMRI” refers to multiparametric Magnetic Resonance Imaging of the prostate.

As used herein the abbreviation “PIRADs” refers to Prostate Imaging Reporting and Data System

### DETAILED DESCRIPTION

The development of reliable, convenient, and accurate tests for the detection of aggressive prostate cancer remains an important objective, particularly during early stages when therapeutic intervention has the highest chance of success. In particular, initial screening procedures such as DRE and PSA are unable to discern between non-aggressive and aggressive prostate cancer effectively. The present invention provides combinations of biomarker/s and clinical variables indicative of aggressive prostate cancer in subjects. In some embodiments the subjects may have previously been determined to have a form of aggressive prostate cancer, or alternatively be suspected of having a form of aggressive prostate cancer on the basis of one or more alternative diagnostic tests (e.g. DRE, PSA testing, MRI). The biomarker/clinical variable combinations may thus be used in various methods and assay formats to differentiate between subjects with aggressive prostate cancer and those who do not have aggressive prostate cancer including, for example, subjects with non-aggressive prostate cancer and subjects who do not have prostate cancer (e.g. subjects with benign prostatic hyperplasia and healthy subjects). The present invention provides for a technical advantage over other available methods in the art. In particular, the biomarker/clinical variable combinations utilised in MiCheck® Prostate can provide for accurate differentiation between subjects with aggressive prostate cancer and those who do not have aggressive prostate cancer in a manner that has previously been unattainable. MiCheck® Prostate can also assist in identifying those patients who may not require a prostate biopsy, or whose biopsy could be delayed, thus providing for a more tailored and streamlined approach to the diagnosis and treatment of prostate cancer.

### Aggressive Prostate Cancer

The present invention provides methods for the detection of aggressive prostate cancer. The methods involve detection of one or more combinations of biomarker/s and clinical variable/s as described herein.

Persons of ordinary skill in the art are well aware of standard clinical tests and parameters used to classify different prostate cancer Gleason grades and Epstein scores (see, for example, “2018 Annual Report on Prostate Diseases”, Harvard Health Publications (Harvard Medical School), 2018; the entire contents of which are incorporated herein by cross-reference).

As known to those of ordinary skill in the art, prostate cancer can be categorized into stages according to the progression of the disease. Under microscopic evaluation, prostate glands are known to spread out and lose uniform structure with increased prostate cancer progression.

By way of non-limiting example, prostate cancer progression may be categorized into stages using the AJCC TNM staging system, the Whitmore-Jewett system and/or the D'Amico risk categories. Ordinarily skilled persons in the field are familiar with such classification systems, their features and their use.

By way of further non-limiting example, a suitable system of grading prostate cancer well known to those of ordinary skill in the field is the “Gleason Grading System”. This system assigns a grade to each of the two largest areas of cancer in tissue samples obtained from a subject with prostate cancer. The grades range from 1-5, 1 being the least aggressive form and 5 the most aggressive form. Metastases are common with grade 4 or grade 5, but seldom occur, for example, in grade 3 tumors. The two grades are then added together to produce a Gleason score. A score of 2-4 is considered low grade; 5-7 intermediate grade; and 8-10 high grade. A tumor with a low Gleason score may typically grow at a slow enough rate to not pose a significant threat to the patient during their lifetime.

As known to those skilled in the art, prostate cancers may have areas with different grades in which case individual grades may be assigned to the two areas that make up most of the prostate cancer. These two grades are added to yield the Gleason score/sum, and in general the first number assigned is the grade which is most common in the tumour. For example, if the Gleason score/sum is written as ‘3+4’, it means most of the tumour is grade 3 and less is grade 4, for a Gleason score/sum of 7.

A Gleason score/sum of 3+4 and above may be indicative of aggressive prostate cancer according to the present invention. Alternatively, a Gleason score/sum of under 3+4 may be indicative of non-aggressive prostate cancer according to the present invention.

An alternative system of grading prostate cancer also known to those of ordinary skill in the field is the “Epstein Grading System”, which assigns overall grade groups ranging from 1-5. A benefit of the Epstein system is assigning a different overall score to Gleason score 7 (3+4) and Gleason score 7 (4+3) since have very different prognoses; Gleason score ‘3+4’ translates to Epstein grade group 2; Gleason score ‘4+3’ translates to Epstein grade group 3.

In other embodiments, Multi-parametric Magnetic Resonance Imaging (mpMRI) may be used in the methods of the present invention, for example, in the initial assessment of patients with suspected prostate cancer (see Tempany et al, 2022. The role of magnetic resonance imaging in prostate cancer. https://www.uptodate.com/contents/the-role-of-magnetic-resonance-imaging-in-prostate-cancer). mpMRI allows visualisation of the prostate and the identification of potentially significant lesions that may represent prostate cancer, or clinically significant prostate cancers. Recent improvements in technology include higher strength magnets and the use of the endorectal coil, although this is not required.

Three imaging sequences are used to generate the mpMRI result:
1. Diffusion weighted imaging (which measures mobility of water molecules)
2. T2 weighted imaging (which reflects local tissue water to allow delineation of the normal prostate anatomy)
3. Dynamic intravenous contrast enhanced imaging (DCE). These images depict the local vascular environment.

According to standard protocols, the imaging results are combined and reported according to the Prostate Imaging Reporting and Data System (PIRADS) classifications developed by the International Prostate MRI Working Group (Hofbauer et al, 2018 Validation of Prostate Imaging Reporting and Data System Version 2 for the Detection of Prostate Cancer. J Urol. 2018; 200(4):767). The PI-RADS system categorizes prostate lesions based on the likelihood of cancer according to a five-point scale, defined as the following:
PI-RADS 1—Clinically significant cancer is highly unlikely to be present.
PI-RADS 2—Clinically significant cancer is unlikely to be present.
PI-RADS 3—The presence of clinically significant cancer is equivocal.
PI-RADS 4—Clinically significant cancer is likely to be present.
PI-RADS 5—Clinically significant cancer is highly likely to be present.

Patients with PIRADS 4 or 5 will typically undergo prostate biopsy due to the high likelihood of detection of aggressive prostate cancer. However, there is no consensus on which PIRADS 3 patients to biopsy and no way of identifying the 18% of PIRADS 1 and 2 patients who have clinically significant prostate cancers. Furthermore, the interpretation of the mpMRI scores varies considerably between operators, with one study demonstrating the significant cancer yield across 9 operators ranging from 3% to 27% for PIRADS 3, from 23% to 65% for PIRADS 4, and from 40% to 80% for PIRADS 5 lesions (Sonn et al, 2019 Prostate Magnetic Resonance Imaging Interpretation Varies Substantially Across Radiologists. Eur. Urol. Focus. 2019; 5(4):592). Therefore, the PIRADs 1-3 group of patients requires development of new technologies to assist clinicians in determining which patients would benefit from proceeding to biopsy and which may be monitored. A test for aggressive prostate cancer can be integrated into the clinical care pathway and have advantages over MRI alone in the following:
1. Decisions to biopsy PIRADs 1, 2 and 3 cancers
2. MRIs can be mis-scored due to operator variability in interpretation of the imaging results
3. Patients can be contraindicated for MRI (contrast allergy, pacemakers, claustrophobia)
4. Biopsy missing cancer due to false negative MRI result.

### Biomarker and Clinical Variable Signatures

In accordance with the methods of the present invention, aggressive prostate cancer can be detected by a combined approach of measuring one or more clinical variables identified herein along with the levels of one or more of the biomarkers identified herein.

A biomarker as contemplated herein may be an analyte. An analyte as contemplated herein is to be given its ordinary and customary meaning to a person of ordinary skill in the art and refers without limitation to a substance or chemical constituent in a biological sample (for example, blood, cerebral spinal fluid, urine, tear/s, lymph fluid, saliva, interstitial fluid, sweat, etc.) that can be detected and quantified. Non-limiting examples include cytokines, chemokines, as well as cell-surface receptors and soluble forms thereof.

A clinical variable as contemplated herein may be associated with or otherwise indicative of prostate cancer (e.g. non-aggressive and/or aggressive forms). The clinical variable may additionally be associated with other disease/s or condition/s. Non-limiting examples of clinical variables relevant to the present invention include subject Age, prostate volume (PV), % free PSA, PSA velocity, PSA density, Prostate Health Index, digital rectal examination (DRE), ethnic background, Age, family history of prostate cancer, prior negative biopsy for prostate cancer.

By way of non-limiting example, a combination of clinical variables and biomarkers according to the present invention can be used for discerning between patients with aggressive forms of prostate cancer and those with non-aggressive forms of prostate cancer or who do not have prostate cancer, and/or for detecting aggressive prostate cancer based on comparisons with a mixed control population of subjects having either non-aggressive prostate cancer or no prostate cancer. The combination of clinical variables and biomarkers may comprise or consist of one, two, three, or more than three individual biomarkers, in combination with one, two, three, or more than three individual clinical variables. The biomarker/s may comprise analytes including, but not limited to WFDC2, % free PSA, free PSA, and/or total PSA.

Without limitation, clinical variable/s, biomarker/s and combinations thereof used for detecting aggressive prostate cancer in accordance with the present invention may comprise or consist of:
WFDC2 (HE4), total PSA, % Free PSA and Age
WFDC2 (HE4), total PSA, % Free PSA, PV and Age
WFDC2 (HE4), total PSA, % Free PSA and PIRADs
WFDC2 (HE4), total PSA, % Free PSA, PV and PIRADs
WFDC2 (HE4), total PSA, % Free PSA, PV, PIRADs and Age
WFDC2 (HE4), total PSA, % Free PSA, PV, PIRADs, Age and DRE
WFDC2 (HE4), total PSA, % Free PSA, Age and DRE
WFDC2 (HE4), total PSA, % Free PSA, Age and FH
WFDC2 (HE4), total PSA, % Free PSA, Age, DRE, FH
WFDC2 (HE4), % Free PSA and Age
WFDC2 (HE4), % Free PSA and PIRADs
WFDC2 (HE4), % Free PSA, PV and Age
WFDC2 (HE4), % Free PSA, PV and PIRADs
WFDC2 (HE4), % Free PSA, PV, Age and PIRADs
WFDC2 (HE4), % Free PSA, PV, Age, PIRADS and DRE
WFDC2 (HE4), total PSA, Free PSA
WFDC2 (HE4), total PSA, Free PSA and PV
WFDC2 (HE4), total PSA, Free PSA, PV and Age
WFDC2 (HE4), total PSA, Free PSA, PV and PIRADs
WFDC2 (HE4), total PSA, Free PSA, PV, PIRADs and Age
WFDC2 (HE4), total PSA, Free PSA and Age
WFDC2 (HE4), total PSA, Free PSA and PIRADs

### Detection and Quantification of Biomarkers

A biomarker or combination of biomarkers according to the present invention may be detected in a biological sample using any suitable method known to those of ordinary skill in the art.

In some embodiments, the biomarker or combination of biomarkers is quantified to derive a specific level of the biomarker or combination of biomarkers in the sample. Level/s of the biomarker/s can be analysed according to the methods provided herein and used in combination with clinical variables to provide a diagnosis.

Detecting the biomarker/s in a given biological sample may provide an output capable of measurement, thus providing a means of quantifying the levels of the biomarker/s present. Measurement of the output signal may be used to generate a figure indicative of the net weight of the biomarker per volume of the biological sample (e.g. pg/mL; μg/mL; ng/mL etc.). Alternatively, measurement of the output signal may be used to generate a figure indicative of the molar amounts per volume of the biological sample (e.g. pmol/mL; pmol/mL; nmol/mL etc.).

By way of non-limiting example only, detection of the biomarker/s may culminate in one or more fluorescent signals indicative of the level of the biomarker/s in the sample. These fluorescent signals may be used directly to make a diagnostic indication according to the methods of the present invention, or alternatively be converted into a different output for that same purpose (e.g. a weight per volume as set out in the paragraph directly above).

Biomarkers according to the present invention can be detected and quantified using suitable methods known in the art including, for example, proteomic techniques and techniques which utilize nucleic acids encoding the biomarkers.

Non-limiting examples of suitable proteomic techniques include mass spectrometry, protein array techniques (e.g. protein chips), gel electrophoresis, and other methods relying on antibodies having specificity for the biomarker/s including immunofluorescence, radiolabelling, immunohistochemistry, immunoprecipitation, Western blot analysis, Enzyme-linked immunosorbent assays (ELISA), fluorescent cell sorting (FACS), immunoblotting, chemiluminescence, and/or other known techniques used to detect protein with antibodies.

Non-limiting examples of suitable techniques relying on nucleic acid detection include those that detect DNA, RNA (e.g. mRNA), cDNA and the like, such as PCR-based techniques (e.g. quantitative real-time PCR; SYBR-green dye staining), UV spectrometry, hybridization assays (e.g. slot blot hybridization), and microarrays.

Antibodies having binding specificity for a biomarker according to the present invention, including monoclonal and polyclonal antibodies, are readily available and can be purchased from a variety of commercial sources (e.g. Sigma-Aldrich, Santa Cruz Biotechnology, Abcam, Abnova, R&D Systems etc.). Additionally or alternatively, antibodies having binding specificity for a biomarker according to the present invention can be produced using standard methodologies in the art. Techniques for the production of hybridoma cells capable of producing monoclonal antibodies are well known in the field. Non-limiting examples include the hybridoma method (see Kohler and Milstein, (1975) Nature, 256:495-497; Coligan et al. section 2.5.1-2.6.7 in Methods In Molecular Biology (Humana Press 1992); and Harlow and Lane Antibodies: A Laboratory Manual, page 726 (Cold Spring Harbor Pub. 1988)), the EBV-hybridoma method for producing human monoclonal antibodies (see Cole, et al. 1985, in Monoclonal Antibodies and Cancer Therapy, Alan R. Liss, Inc., pp. 77-96), the human B-cell hybridoma technique (see Kozbor et al. 1983, Immunology Today 4:72), and the trioma technique.

In some embodiments, detection/quantification of the biomarker/s in a biological sample (e.g. a body fluid or tissue sample) is achieved using an Enzyme-linked immunosorbent assay (ELISA). The ELISA may, for example, be based on colourimetry, chemiluminescence, and/or fluorometry. An ELISA suitable for use in the methods of the present invention may employ any suitable capture reagent and detectable reagent including antibodies and derivatives thereof, protein ligands and the like.

By way of non-limiting example, in a direct ELISA the biomarker of interest can be immobilized by direct adsorption onto an assay plate or by using a capture antibody attached to the plate surface. Detection of the antigen can then be performed using an enzyme-conjugated primary antibody (direct detection) or a matched set of unlabeled primary and conjugated secondary antibodies (indirect detection). The indirect detection method may utilise a labelled secondary antibody for detection having binding specificity for the primary antibody. The capture (if used) and/or primary antibodies may derive from different host species.

In some embodiments, the ELISA is a competitive ELISA, a sandwich ELISA, an in-cell ELISA, or an ELISPOT (enzyme-linked immunospot assay).

Methods for preparing and performing ELISAs are well known to those of ordinary skill in the art. Procedural considerations such as the selection and coating of ELISA plates, the use of appropriate antibodies or probes, the use of blocking buffers and wash buffers, the specifics of the detection step (e.g. radioactive or fluorescent tags, enzyme substrates and the like), are well established and routine in the field (see, for example, “The Immunoassay Handbook. Theory and applications of ligand binding, ELISA and related techniques”, Wild, D. (Ed), 4^{th} edition, 2013, Elsevier).

In other embodiments, detection/quantification of the biomarker/s in a biological sample (e.g. a body fluid or tissue sample) is achieved using Western blotting. Western blotting is well known to those of ordinary skill in the art (see for example, Harlow and Lane. Using antibodies. A Laboratory Manual. Cold Spring Harbor, New York: Cold Spring Harbor Laboratory Press, 1999; Bold and Mahoney, Analytical Biochemistry 257, 185-192, 1997). Briefly, antibodies having binding affinity to a given biomarker can be used to quantify the biomarker in a mixture of proteins that have been separated based on size by gel electrophoresis. A membrane made of, for example, nitrocellulose or polyvinylidene fluoride (PVDF) can be placed next to a gel comprising a protein mixture from a biological sample and an electrical current applied to induce the proteins to migrate from the gel to the membrane. The membrane can then be contacted with antibodies having specificity for a biomarker of interest, and visualized using secondary antibodies and/or detection reagents.

In other embodiments, detection/quantification of multiple biomarkers in a biological sample (e.g. a body fluid or tissue sample) is achieved using a multiplex protein assay (e.g. a planar assay or a bead-based assay). There are numerous multiplex protein assay formats commercially available (e.g. Bio-rad, Luminex, EMD Millipore, R&D Systems), and non-limiting examples of suitable multiplex protein assays are described in the Examples section of the present specification.

In other embodiments, detection/quantification of biomarker/s in a biological sample (e.g. a body fluid or tissue sample) is achieved by flow cytometry, which is a technique for counting, examining and sorting target entities (e.g. cells and proteins) suspended in a stream of fluid. It allows simultaneous multiparametric analysis of the physical and/or chemical characteristics of entities flowing through an optical/electronic detection apparatus (e.g. target biomarker/s quantification).

In other embodiments, detection/quantification of biomarker/s in a biological sample (e.g. a body fluid or tissue sample) is achieved by immunohistochemistry or immunocytochemistry, which are processes of localizing proteins in a tissue section or cell, by use of antibodies or protein binding agent having binding specificity for antigens in tissue or cells. Visualization may be enabled by tagging the antibody/agent with labels that produce colour (e.g. horseradish peroxidase and alkaline phosphatase) or fluorescence (e.g. fluorescein isothiocyanate (FITC) or phycoerythrin (PE)).

Persons of ordinary skill in the art will recognize that the particular method used to detect biomarker/s according to the present invention or nucleic acids encoding them is a matter of routine choice that does not require inventive input.

### Measurement of Clinical Variables

A clinical variable or a combination of clinical variables according to the present invention may be assessed/measured/quantified using any suitable method known to those of ordinary skill in the art.

In some embodiments, the clinical variable/s may comprise relatively straightforward parameter/s (e.g. age) accessible, for example, via medical records.

In other embodiments, the clinical variable/s may require assessment by medical and/or other methodologies known to those of ordinary skill in the art. For example, prostate volume may require measurement by techniques using ultrasound (e.g. transabdominal ultrasonography, transrectal ultrasonography), magnetic resonance imaging, and the like. DRE results are typically scored as normal or abnormal/suspicious.

Clinical variable/s relevant to the diagnostic methods of the present invention may be assessed, measured, and/or quantified using additional or alternative methods including, by way of example, digital rectal exam, biopsy and/or mpMRI fusion (from which both PIRADs score and prostate volume can be derived).

Clinical variable/s such as PSA level, free PSA, total PSA, % free PSA, WFDC2(HE4) may be determined by use of clinical immunoassays such as the Beckman Coulter Access 2 analyzer and associated Hybritech assays, Roche Cobas, Abbott Architect, Abbott Alinity or other similar assays.

### Analysis of Biomarkers, Clinical Variables and Diagnosis

According to methods of the present invention, the assessment of a given combination of clinical variable/s and biomarker/s may be used as a basis to diagnose aggressive prostate cancer, or determine an absence of aggressive prostate cancer in a subject of interest.

In relation to assessing biomarker component/s of the combination, the methods generally involve analyzing the targeted biomarker/s in a given biological sample or a series of biological samples to derive a quantitative measure of the biomarker/s in the sample. Suitable biomarker/s include, but are not limited to, those biomarkers and biomarker combinations referred to above in the section entitled “Biomarker and clinical variable signatures”, and the Examples of the present application. By way of non-limiting example only, the quantitative measure may be in the form of a fluorescent signal or an absorbance signal as generated by an assay designed to detect and quantify the biomarker/s. Additionally or alternatively, the quantitative measure may be provided in the form of weight/volume or moles/volume measurements of the biomarker/s in the sample/s.

Similarly, in relation to assessing clinical variable component/s of the combination, assessment of feature/s such as, for example, subject age and/or prostate volume can be made and a representative value generated (e.g. a numerical value). Suitable clinical variable/s include, but are not limited to, those clinical variable/s referred to above in the section entitled “Biomarker and clinical variable signatures”, and the Examples of the present application.

In some embodiments, the methods of the present invention may comprise a comparison of levels of the biomarker/s and clinical variable/s in patient populations known to suffer from aggressive prostate cancer, known to suffer from non-aggressive cancer, or known not to suffer from prostate cancer (e.g. benign prostatic hyperplasia patient populations and/or healthy patient populations). For example, levels of biomarker/s and measures of clinical variable/s can be ascertained from a series of biological samples obtained from patients having an aggressive prostate cancer compared to patients having a non-aggressive prostate cancer and/or no cancer. Aggressive prostate cancer may be characterized by a minimum Gleason grade or score/sum (e.g. at least 7 (e.g. 3+4 or 4+3, 5+2), or at least 8 (e.g. 4+4, 5+3 or 3+5).

The level of biomarker/s observed in samples from each individual population and clinical variable/s of the individuals within each population may be collectively analysed to determine a threshold value that can be used as a basis to provide a diagnosis of aggressive prostate cancer, or an absence of aggressive prostate cancer. For example, a biological sample from a patient confirmed or suspected to be suffering from aggressive prostate cancer can be analysed and the levels of target biomarker/s according to the present invention determined in combination with an assessment of clinical variable/s. Comparison of levels of the biomarker/s and the clinical variable/s in the patient's sample to the threshold value/s generated from the patient populations can serve as a basis to detect aggressive prostate cancer or an absence of aggressive prostate cancer.

Accordingly, in some embodiments the methods of the present invention comprise diagnosing whether a given patient suffers from aggressive prostate cancer. The patient may have been previously confirmed to have or suspected of having prostate cancer, for example, as a result of a MRI, DRE and/or PSA test. In such situations, it is advantageous for the patient to determine whether the patient is likely to have aggressive prostate cancer or not, in accordance with the methods described herein avoiding the need for a prostate biopsy. In some embodiments, a patient may have previously received a PIRADs score of 1-5, or a PIRADs score of 1, 2 or 3.

Without any particular limitation, a diagnostic method according to the present invention may involve discerning whether a subject has or does not have aggressive prostate cancer. The method may comprise obtaining a first series of biological samples from a first group of patients biopsy-confirmed to be prostate cancer free or suffering from non-aggressive prostate cancer, and a second series of biological samples from a second group of patients biopsy-confirmed to be suffering from aggressive prostate cancer. A threshold value for discerning between the first and second patient groups may be generated by measuring clinical variable/s such as subject age and/or prostate volume and/or DRE status and/or PIRADs score and detecting levels/concentrations of one, two, three, four, five or more than five biomarkers in the first and second series of biological samples to thereby obtain a biomarker level for each biomarker in each biological sample of each series. Clinical variables prostate volume and MRI PIRADs score are considered “variables” in determining the presence or absence of aggressive prostate cancer. The variables may be combined in a manner that allows discrimination between samples from the first and second group of patients. A threshold value or probability score may be selected from the combined variable values in a suitable manner such as any one or more of a method that: reduces the misclassification rate between the first and second group of patients; increases or maximizes the sensitivity in discriminating between the first and second group of patients; and/or increases or maximizes the specificity in discriminating between the first and second group of patients; and/or increases or maximises the accuracy in discriminating between the first and second group of patients. A suitable algorithm and/or transformation of individual or combined variable values obtained from the test subject and its biological sample may be used to generate the variable values for comparison to the threshold value. In some embodiments, one or more variables used in deriving the threshold value and/or the test subject score are weighted.

In some embodiments, the subject may receive a negative indication for aggressive prostate cancer if the subject's score generated from the combined biomarker level/s and clinical variable/s is less than the threshold value. In some embodiments, the subject receives a positive diagnosis for aggressive prostate cancer if the subject's score generated from the combined biomarker level/s and clinical variable/s is less than the threshold value. In some embodiments, the subject receives a negative diagnosis for aggressive prostate cancer if the subject's score generated from the combined biomarker level/s and clinical variable/s is more than the threshold value. In some embodiments, the patient receives a positive diagnosis for aggressive prostate cancer if the subject's score generated from the combined biomarker level/s and clinical variable/s is more than the threshold value.

Suitable and non-limiting methods for conducting these analyses are described in the Examples of the present application.

One non-limiting example of such a method is Receiver Operating Characteristic (ROC) curve analysis. Generally, the ROC analysis may involve comparing a classification for each patient tested to a ‘true’ classification based on an appropriate reference standard.

Classification of multiple patients in this manner may allow derivation of measures of sensitivity and specificity. Sensitivity will generally be the proportion of correctly classified patients among all of those that are truly positive, and specificity the proportion of correctly classified cases among all of those that are truly negative. In general, a trade-off may exist between sensitivity and specificity depending on the threshold value selected for determining a positive classification. A low threshold may generally have a high sensitivity but relatively low specificity. In contrast, a high threshold may generally have a low sensitivity but a relatively high specificity. A ROC curve may be generated by inverting a plot of sensitivity versus specificity horizontally. The resulting inverted horizontal axis is the false positive fraction, which is equal to the specificity subtracted from 1. The area under the ROC curve (AUC) may be interpreted as the average sensitivity over the entire range of possible specificities, or the average specificity over the entire range of possible sensitivities. The AUC represents an overall accuracy measure and also represents an accuracy measure covering all possible interpretation thresholds.

While methods employing an analysis of the entire ROC curve are encompassed, it is also intended that the methods may be extended to statistical analysis of a partial area (partial AUC analysis). The choice of the appropriate range along the horizontal or vertical axis in a partial AUC analysis may depend at least in part on the clinical purpose. In a clinical setting in which it is important to detect the presence of aggressive prostate cancer with high accuracy, a range of relatively high false positive fractions corresponding to high sensitivity (low false negatives) may be used. Alternatively, in a clinical setting in which it is important to exclude the presence of aggressive prostate cancer, a range of relatively low false positive fractions equivalent to high specificities (high true positives) may be used.

Subjects, Samples and Controls A subject or patient referred to herein encompasses any animal of economic, social or research importance including bovine, equine, ovine, canine, primate, avian and rodent species.

A subject or patient may be a mammal such as, for example, a human or a non-human mammal.

Subjects and patients as described herein may or may not suffer from aggressive prostate cancer, or may or may not suffer from a non-aggressive prostate cancer.

In accordance with methods of the present invention, clinical variable/s of a given subject may be assessed and the output combined with levels of biomarker/s measured in a sample from the subject.

A sample used in accordance the methods of the present invention may be in a form suitable to allow analysis by the skilled artisan. Suitable samples include various body fluids such as blood, plasma, serum, semen, urine, tear/s, cerebral spinal fluid, lymph fluid, saliva, interstitial fluid, sweat, etc. The urine may be obtained following massaging of the prostate gland.

The sample may be a tissue sample, such as a biopsy of the tissue, or a superficial sample scraped from the tissue. The tissue may be from the prostate gland. In another embodiment the sample may be prepared by forming a suspension of cells made from the tissue.

The methods of the present invention may, in some embodiments, involve the use of control samples.

A control sample is any corresponding sample (e.g. tissue sample, blood, plasma, serum, semen, tear/s, or urine) that is taken from an individual without aggressive prostate cancer. In certain embodiments, the control sample may comprise or consist of nucleic acid material encoding a biomarker according to the present invention.

In some embodiments, the control sample can include a standard sample. The standard sample can provide reference amounts of biomarker at levels considered to be control levels. For example, a standard sample can be prepared to mimic the amounts or levels of a biomarker described herein in one or more samples (e.g. an average of amounts or levels from multiple samples) from one or more subjects, who may or may not have aggressive prostate cancer.

In some embodiments control data may be utilized. Control data, when used as a reference, can comprise compilations of data, such as may be contained in a table, chart, graph (e.g. database or standard curve) that provide amounts or levels of biomarker/s and/or clinical variable feature/s considered to be control levels. Such data can be compiled, for example, by obtaining amounts or levels of the biomarker in one or more samples (e.g. an average of amounts or levels from multiple samples) from one or more subjects, who may or may not have aggressive prostate cancer. Clinical variable control data can be obtained by assessing the variable in one or more subjects who may or may not have aggressive prostate cancer.

### Treatment

Once the biomarker and clinical variable combinations of the invention have confirmed the presence of aggressive prostate cancer in accordance with the methods described herein, the present invention further contemplates treating the aggressive prostate cancer in a subject in need thereof. In this context, the method will typically involve biopsy of the prostate to confirm aggressive prostate cancer. A suitable treatment will then be assigned to the patient based on the histopathological analysis of the biopsy and/or the knowledge of a skilled person in the art.

In one embodiment, the treatment includes one or more of surgery, chemotherapy, radiation therapy, immunotherapy, hormone therapy or drug treatment.

In another embodiment, the treatment includes one or more drugs selected from the group consisting of an anti-androgenic agent (e.g. Abiraterone Acetate, Apalutamide, Bicalutamide, Daralutomide, Enzalutamide, Flutamide, Nilutamide), an alkylating agent (e.g., Cisplatin, Carboplatin, Oxaliplatin, BBR3464, Chlorambucil, Chlormethine, Cyclophosphamides, Ifosfamide, Melphalan, Carmustine, Fotemustine, Lomustine, Streptozocin, Busulfan, Dacarbazine, Mechlorethamine, Procarbazine, Temozolomide, ThioTPA, and Uramustine); a GnRH agonist/antagonist (e.g. Degarelix, Leuprolide Acetate, relugolix), an LHRH agonist/antagonist (e.g. Gosrelin acetate), an anti-metabolite (e.g., purine (azathioprine, mercaptopurine), pyrimidine (Capecitabine, Cytarabine, Fluorouracil, Gemcitabine), and folic acid (Methotrexate, Pemetrexed, Raltitrexed)); a vinca alkaloid (e.g., Vincristine, Vinblastine, Vinorelbine, Vindesine); a taxane (e.g., paclitaxel, docetaxel, BMS-247550, cabazitaxel); an anthracycline (e.g., Daunorubicin, Doxorubicin, Epirubicin, Idarubicin, Mitoxantrone, Valrubicin, Bleomycin, Hydroxyurea, and Mitomycin); a topoisomerase inhibitor (e.g., Topotecan, Irinotecan Etoposide, and Teniposide); a monoclonal antibody (e.g., Alemtuzumab, Bevacizumab, Cetuximab, Gemtuzumab, Panitumumab, Rituximab, and Trastuzumab); a photosensitizer (e.g., Aminolevulinic acid, Methyl aminolevulinate, Porfimer sodium, and Verteporfin); a tyrosine kinase inhibitor (e.g., Gleevec™); an epidermal growth factor receptor inhibitor (e.g., Iressa™, erlotinib (Tarceva™), gefitinib); an FPTase inhibitor (e.g., FTIs (R1 15777, SCH66336, L-778,123)); a KDR inhibitor (e.g., SU6668, PTK787); a proteosome inhibitor (e.g., PS341); a TS/DNA synthesis inhibitor (e.g., ZD9331, Raltirexed (ZD 1694, Tomudex), ZD9331, 5-FU)); an S-adenosyl-methionine decarboxylase inhibitor (e.g., SAM468A); a DNA methylating agent (e.g., TMZ); a DNA binding agent (e.g., PZA); an agent which binds and inactivates 0—alkylguanine AGT (e.g., BG); a z-raf-X antisense oligo-deoxynucleotide (e.g., ISIS-5132 (CGP-69846A)); tumor immunotherapy; a radio labelled agent (e.g. Lutetium Lu 177 Vipivotide Tetraxetan, radium 223, Strontium 89 or samarium 153), a PARP inhibitor (e.g. olaparib, rucaparib camsylate, talazoparib tosylate) a steroidal and/or non-steroidal anti-inflammatory agent (e.g., corticosteroids, COX-2 inhibitors); or other agents such as Alitretinoin, Altretamine, Amsacrine, Anagrelide, Arsenic trioxide, Asparaginase, Bexarotene, Bortezomib, Celecoxib, Dasatinib, Denileukin Diftitox, Estramustine, Hydroxycarbamide, Imatinib, Pentostatin, Masoprocol, Mitotane, Pegaspargase, and Tretinoin.

Preferable treatments for a subject diagnosed with aggressive prostate cancer will depend on the tumour grade, any metastases present and the patient's life expectancy and can include active surveillance, radical prostatectomy, external beam radiation therapy or brachytherapy (with or without concomitant androgen deprivation therapy), or combinations thereof as outlined in the National Comprehensive Cancer Centre Prostate Cancer Guidelines (2022).

Suitable treatments may also be determined according to a risk score as determined by the Gleason score. For example, an intermediate-risk group would typically have a Gleason score of 7 (primary 3+secondary 4) or (primary 4+secondary 3), and a high/very high risk group would have a Gleason score of 8-10.

If a subject has been diagnosed as being of intermediate risk for aggressive prostate cancer, radiation therapy (external beam or brachytherapy), often with hormone therapy, may be suitable. A radical prostatectomy with pelvic lymph node dissection (PLND) is also an option. Depending on the findings from a biopsy, treatments might also include:
external beam radiation therapy with or without hormone therapy if the cancer is found in the lymph nodes or if it has features that make it more likely to recur;
active surveillance for people whose cancers have favorable features.

If a subject has been diagnosed as being at high risk for aggressive prostate cancer, treatments may include:
radiation therapy (external beam with brachytherapy or external beam radiation alone) along with hormone therapy for 1 to 3 years;
radical prostatectomy with PLND.

If cancer is found in the lymph nodes removed during surgery or if it has features that make it more likely to recur, hormone therapy with or without radiation might be suitable.

If a subject has been diagnosed as being of very high risk for aggressive prostate cancer, treatments may include:
external beam radiation therapy (with or without brachytherapy) along with hormone therapy (ADT) for 1 to 3 years. Sometimes, the chemotherapy drug docetaxel or the hormone drug abiraterone might be added to radiation plus ADT;
radical prostatectomy with PLND.

If cancer is found in the lymph nodes removed during surgery, hormone therapy with or without external beam radiation treatment might be given. Radiation therapy with or without hormone therapy might be recommended if the cancer is not found in the lymph nodes but does have features that make it more likely to recur.

If a subject has been diagnosed with Stage IVA prostate cancer, having spread to nearby lymph nodes but not to distant parts of the body, treatment options include:
external beam radiation treatment with hormone therapy (ADT, with or without abiraterone);
hormone therapy (ADT, with or without abiraterone);
radical prostatectomy with PLND.

If cancer is found in the lymph nodes removed during surgery or it has features that make it more likely to recur, hormone therapy with or without external beam radiation treatment might be given.

If a subject has been diagnosed with Stage IVB prostate cancer, meaning the prostate cancer has spread to distant organs such as the bones, treatment options may include:
hormone therapy (typically ADT, alone or along with a newer hormone drug);
hormone therapy with chemotherapy (usually docetaxel);
hormone therapy with external beam radiation to the tumor in the prostate;
surgery to relieve symptoms such as bleeding or urinary obstruction;
observation (for those who are older or have other serious health issues and do not have major symptoms from the cancer);
clinical trial participation.

Treatment of stage IV prostate cancer may also include treatments to help prevent or relieve symptoms such as pain from bone metastases. This can be done with external radiation or with drugs like denosumab (Xgeva), a bisphosphonate like zoledronic acid (Zometa), or a radiopharmaceutical such as radium-223, strontium-89, or samarium-153.

If the cancer continues to grow and spread or if it recurs, other treatments might be options, such as immunotherapy, targeted drug therapy, chemotherapy, or other forms of hormone therapy.

The present invention also contemplates the treatment of a subject identified as not having aggressive prostate cancer. Typically, these subjects have a Gleason score of 3+3 or do not have prostate cancer.

In one embodiment, if a subject has been determined to have non-aggressive prostate cancer, treatment options include observation, active surveillance, radiation therapy (external beam or brachytherapy) or radical prostatectomy and surgery. These treatment regimens may be carried out with or without hormone therapy.

The existence of, improvement in, or treatment of, aggressive prostate cancer may be determined by any clinically or biochemically relevant method as described herein or known in the art. Other indicators of a positive response to treatment may be assessed and include: less difficulty in urinating, reduced or absent blood in semen, less or absent pain in pelvic area, reduced or absent bone pain, reduced or absent urinary incontinence, and reduced or absent erectile dysfunction.

### Kits

Also contemplated herein are kits for performing the methods of the present invention.

The kits may comprise reagents suitable for detecting one or more biomarker/s described herein, including, but not limited to, those biomarker and biomarker combinations referred to in the section above entitled “Biomarker and clinical variable signatures”.

By way of non-limiting example, the kits may comprise one or a series of antibodies capable of binding specifically to one or a series of biomarkers described herein.

Additionally or alternatively, the kits may comprise reagents and/or components for determining clinical variable/s of a subject (e.g. PSA levels), and/or for preparing and/or conducting assays capable of quantifying one or more biomarker/s described herein (e.g. reagents for performing an ELISA, multiplex bead-based Luminex assay, flow cytometry, Western blot, immunohistochemistry, gel electrophoresis (as suitable for protein and/or nucleic acid separation) and/or quantitative PCR. Such assays may be performed using systems such as the Roche Cobas, Abbott Architect or Alinity, or Beckmann Coulter Access 2 analyzer and associated Hybritech assays.

Additionally or alternatively, the kits may comprise equipment for obtaining and/or processing a biological sample as described herein, from a subject.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be described with reference to specific example(s), which should not be construed as in any way limiting.

### Example 1: Background & Study Design

### 1.1 Clinical Diagnostic Pathway—No MRI

A flow diagram depicting a clinical diagnostic pathway for aggressive prostate cancer is shown in the schematic below. In this instance, MRI is not being used for the treatment pathway. This could be due to the patient not having access to an MRI or being in-eligible for MRI (for instance having contrast allergy, metal implants or severe claustrophobia).

In brief:
1. Primary care physician refers patient with raised PSA result to a urologist.
2. Urologist repeats PSA test.
3. If above the age-adjusted PSA cut-off, the patient proceeds to biopsy.
4. If the biopsy shows a Gleason score 3+4 (or above) treatment with various modalities such as surgery, radiation, drugs is initiated.
5. If biopsy shows Gleason score of 3+3 physician may consider transperineal biopsy, MRI or active surveillance.

The flow diagram below outlines an exemplary strategy for implementation of the diagnostic methods of the present invention.

Briefly:
1. The primary care physician refers patient with raised PSA result to a urologist.
2. The urologist repeats PSA and performs diagnostic method according to the present invention.
3. If the method provides a ‘no aggressive cancer’ determination the patient does not proceed to biopsy but is followed up in 3-6 months, with possible biopsy at 1 year.
4. If the method provides an aggressive diagnosis the urologist orders a biopsy. If the biopsy shows Gleason score 3+4 (or above) treat with various modalities such as surgery, radiation, drugs.
5. If the biopsy shows Gleason score of 3+3 a transperineal biopsy, MRI or active surveillance can be considered.

### 1.2 Clinical Diagnostic Pathway—Post MRI

A flow diagram depicting a clinical diagnostic pathway for aggressive prostate cancer is shown in the schematic below. In this instance, the patient is referred to MRI following a raised PSA.

In brief:
1. Primary care physician refers patient with raised PSA result to a urologist.
2. Urologist performs an MRI.
3. If the MRI PIRADS score is either a 4 or 5, the patient will typically proceed to biopsy.
4. If the MRR PIRADS score is a 1 or 2, the patient will typically be monitored
5. If the MRI PIRADs score is 3, the urologist may recommend a biopsy, or may recommend the patient is monitored.

The flow diagram below outlines an exemplary strategy for implementation of the diagnostic methods of the present invention.

Briefly:
1. The primary care physician refers patient with raised PSA result to a urologist.
2. The urologist orders an MRI scan.
3. If the MRI PIRADS score is either a 4 or 5, the patient will typically proceed to biopsy. Alternatively, the urologist may choose to order the diagnostic method according to the present invention.
4. If the MRI PIRADS score is a 1, 2 or 3 the physician orders the diagnostic method according to the present invention.
3. If the method provides a ‘no aggressive cancer’ determination the patient does not proceed to biopsy but is followed up in 3-6 months.
5. If the method provides an aggressive determination the urologist orders a biopsy.

### 1.3 Clinical Diagnostic Pathway—Pre- and Post-MRI

A flow diagram depicting a clinical diagnostic pathway for aggressive prostate cancer is shown in the schematic below. In this instance, the present invention is being used to firstly determine whether a patient should have an MRI (pre-MRI). This would be used in when a patient did not have easy access to an MRI (e.g. they are remotely located and would need to travel to a center with an MRI) or where the patient is not re-imbursed for the costs of the MRI and hence provides an indication as to whether an MRI should be performed. Once the MRI has been performed, the present invention can be used to determine whether to proceed to prostate biopsy (post-MRI).

Briefly:
1. The primary care physician refers patient with raised PSA result to a urologist.
2. The urologists orders the diagnostic method according to the present invention.
3. Following an indication of aggressive cancer, the urologist orders an MRI scan.
4. If the MRI PIRADS score is either a 4 or 5, the patient will typically proceed to biopsy. Alternatively, the physician may choose to order the diagnostic method according to the present invention.
5. If the MRI PIRADS score is a 1, 2 or 3 the physician orders the diagnostic method according to the present invention.
6. If the method provides a ‘no aggressive cancer’ determination the patient does not proceed to biopsy but is followed up in 3-6 months.
7. If the method provides an aggressive determination the urologist orders a biopsy.

### 1.4 Overview of Model Development

A summary of the strategy used to identify model components follows below:
Samples were collected from a representative contemporary US patient population (‘CUSP’ prospective trial) and a representative Australian population (Macquarie Trial).
Samples were measured using current prostate cancer diagnosis tests: PSA, % free PSA and the WFDC2 (HE4) test previously identified as biomarker able to contribute to models differentiating aggressive CaP from NOT-Aggressive CaP.
Measurements of clinical variables used in risk calculators were made (age, ethnic background, PSA, DRE, prostate volume, family history, prior biopsy results) for the US cohort, and (age, PSA, DRE, prostate volume, family history, PIRADs scores) for the Australian cohort.
The performance of clinical tests/factors at differentiating aggressive vs NOT-aggressive CaP in these cohorts were determined.
Models were developed using existing clinical tests/factors and adding one or more biomarker markers (note this approach minimizes the number of new markers that need to be added to existing tests).

### 1.5 Australian Patient Cohort and Trial Parameters

A prospective study was conducted at Macquarie University Hospital (MUH, Ethics approval 5201500707). Serum samples were collected from patients undergoing prostate biopsy for suspicion of prostate cancer. Blood markers were measured at Douglas Hanley Moir (DHM) laboratories using Abbott Architect and Alinity immunoassays. Samples were classified as either; no cancer (No CaP), non-aggressive cancer (GS3+3, Non-AgCaP) or aggressive cancer (AgCaP) based on the patient's biopsy result. The patient's PIRAD classification and family history of prostate cancer was obtained from their medical record. The prostate volume was obtained from the MRI result (187 patients) or from by TRUS (5 patients).

Overall, 78/192 (40.6%) of patients had no prostate cancer on biopsy with 114 (58.4%) having cancer. 77 (40.1%) patients had aggressive prostate cancer and 37 (32.4%) had non-aggressive prostate cancer.

PIRADs data was available for 184 patients, with 8 (4.2%) either not eligible for MRI or for whom MRI was not performed due to clinician's recommendation. The PIRADs scores for the remaining patients were 2 PIRADs 1 (1%), 16 PIRADS 2 (8%), 23 PIRADs 3 (12%), 88 PIRADs 4 (46%) and 55 PIRADs 5 patients (29%).

Study patient characteristics are outlined in Table 1 below.

TABLE 1
Patient characteristics for Macquarie cohort

| | | | | | P Value | Not AgCaP | | | |
|---|---|---|---|---|---|---|---|---|---|
| | | | | | (non-CaP | (No CaP + | | | |
| | No Cancer | | Cancer | | v CaP) | NonAgCaP) | | AgCaP | |
| Total | 78 | (40.6%) | 114 | (59.4%) | | 115 | (59.9%) | 77 | (40.1%) |
| Age | | | | | <0.01 | | | | |
| Mean (SD) | 62.82 | (7.81) | 66.33 | (8.59) | | 63.10 | (8.12) | 67.61 | (8.22) |
| Median | 63.5 | (43-82) | 66.5 | (36-84) | | 64 | (36-82) | 68 | (49-84) |
| (Range) | | | | | | | | | |
| >50 years, | 73 | (93.6%) | 109 | (95.6%) | | 107 | (93.0%) | 75 | (97.4%) |
| N(%) | | | | | | | | | |
| Prostate | | | | | <0.01 | | | | |
| Volume (cc) | | | | | | | | | |
| N measured | 78 | (100.0%) | 114 | (100.0%) | | 115 | (100.0%) | 77 | (100.0%) |
| (%) | | | | | | | | | |
| Ultrasound | — | | 5 | | | 4 | | 1 | |
| MRI | 78 | | 109 | | | 111 | | 76 | |
| Mean (SD) | 59.29 | (21.12) | 51.14 | (24.18) | | 60.83 | (24.91) | 44.93 | (16.67) |
| Median | 59.00 (22.00- | | 47.00 (7.00- | | | 59.00 (18.00- | | 43.00 (7.00- | |
| (Range) | 112.86) | | 150.00) | | | 150.00) | | 92.00) | |
| Enrolling PSA | | | | | 0.09 | | | | |
| (ng/ml) | | | | | | | | | |
| Mean (SD) | 5.68 | (3.33) | 7.59 | (12.99) | | 5.87 | (3.35 | 8.24 | (15.63) |
| Median | 4.85 (1.70- | | 5.40 (0.38- | | | 4.90 (1.00- | | 6.00 (0.38- | |
| (Range) | 25.00) | | 140.00) | | | 25.00) | | 140.00) | |
| First Degree | | | | | 0.19 | | | | |
| Family | | | | | | | | | |
| History, N (%) | | | | | | | | | |
| Yes | 5 | (6.4%) | 17 | (14.9%) | | 10 | (8.7%) | 12 | (15.6%) |
| No | 45 | (57.7%) | 60 | (52.6%) | | 66 | (57.4%) | 39 | (50.6%) |
| Unknown | 28 | (35.9%) | 37 | (32.5%) | | 39 | (33.9%) | 26 | (33.8%) |
| DRE status | | | | | 0.55 | | | | |
| Normal | 40 | (51.3%) | 63 | (55.3%) | | 65 | (56.5%) | 38 | (49.4%) |
| Suspicious | 17 | (21.8%) | 28 | (24.6%) | | 23 | (20.0%) | 22 | (28.6%) |
| Unknown | 21 | (26.9%) | 23 | (20.2%) | | 27 | (23.5%) | 17 | (22.1%) |
| Gleason | | | | | | | | | |
| Score/Epstein, | | | | | | | | | |
| N (%) | | | | | | | | | |
| 6/1 | | | 34 | (32.5%) | | 37 | (32.2%) | | |
| 7 (3 + 4)/2 | | | 43 | (37.7%) | | | | 43 | (55.8%) |
| 7 (4 + 3)/3 | | | 20 | (14.5%) | | | | 20 | (26.0%) |
| 8/4* | | | 4 | (3.5%) | | | | 4 | (5.2%) |
| 9/5# | | | 10 | (8.8%) | | | | 10 | (13.0%) |
| PIRADS (%) | | | | | | | | | |
| No MRI | 2 | (2.6%) | 6 | (5.3%) | | 6 | (5.2%) | 2 | (2.6%) |
| information | | | | | | | | | |
| 1 | 2 | (2.6%) | 0 | (0.0%) | | 2 | (1.7%) | 0 | (0.0%) |
| 2 | 13 | (16.7%) | 3 | (2.6%) | | 15 | (13.0%) | 1 | (1.3%) |
| 3 | 10 | (12.8%) | 13 | (11.4%) | | 14 12.2%) | | 9 | (11.7%) |
| 4 | 35 | (44.9%) | 53 | (46.5%) | | 55 | (47.8%) | 33 | (42.9%) |
| 5 | 16 | (20.5%) | 39 | (34.2%) | | 23 | (20.0%) | 32 | (41.6%) |

| | P Value | | | | | P Value |
|---|---|---|---|---|---|---|
| | (Not AgCaP | | | | | (Non-AgCaP |
| | v AgCaP) | Non-Ag CaP | | AgCaP | | v AgCaP) |
| Total | | 37 | (32.4%) | 77 | (67.6%) | |
| Age | <0.01 | | | | | 0.03 |
| Mean (SD) | | 63.68 | (8.83) | 67.61 | (8.22) | |
| Median | | 64 | (36-80) | 68 | (49-84) | |
| (Range) | | | | | | |
| >50 years, | | 34 | (91.9%) | 75 | (97.4%) | |
| N(%) | | | | | | |
| Prostate | <0.01 | | | | | <0.01 |
| Volume (cc) | | | | | | |
| N measured | | 37 | (100.0%) | 77 | (100.0%) | |
| (%) | | | | | | |
| Ultrasound | | 4 | | 1 | | |
| MRI | | 33 | | 76 | | |
| Mean (SD) | | 64.07 | (31.53) | 44.93 | (16.67) | |
| Median | | 55.00 (18.00- | | 43.00 (7.00- | | |
| (Range) | | 150.00) | | 92.00) | | |
| Enrolling PSA | 0.13 | | | | | 0.59 |
| (ng/ml) | | | | | | |
| Mean (SD) | | 6.25 | (3.42) | 8.24 | (15.63) | |
| Median | | 5.10 (1.00- | | 6.00 (0.38- | | |
| (Range) | | 17.00) | | 140.00) | | |
| First Degree | 0.32 | | | | | 0.83 |
| Family | | | | | | |
| History, N (%) | | | | | | |
| Yes | | 5 | (13.5%) | 12 | (15.6%) | |
| No | | 21 | (56.8%) | 39 | (50.6%) | |
| Unknown | | 11 | (29.7%) | 26 | (33.8%) | |
| DRE status | 0.38 | | | | | 0.18 |
| Normal | | 25 | (67.6%) | 38 | (49.4%) | |
| Suspicious | | 6 | (16.2%) | 22 | (28.6%) | |
| Unknown | | 6 | (16.2%) | 17 | (22.1%) | |
| Gleason | | | | | | |
| Score/Epstein, | | | | | | |
| N (%) | | | | | | |
| 6/1 | | 37 | (100%) | | | |
| 7 (3 + 4)/2 | | | | 43 | (55.8%) | |
| 7 (4 + 3)/3 | | | | 20 | (26.0%) | |
| 8/4* | | | | 4 | (5.2%) | |
| 9/5# | | | | 10 | (13.0%) | |
| PIRADS (%) | | | | | | |
| No MRI | | 4 | (10.8%) | 2 | (2.6%) | |
| information | | | | | | |
| 1 | | 0 | (0.0%) | 0 | (0.0%) | |
| 2 | | 2 | (5.4%) | 1 | (1.3%) | |
| 3 | | 4 | (10.8%) | 9 | (11.7%) | |
| 4 | | 20 | (54.1%) | 33 | (42.9%) | |
| 5 | | 7 | (18.9%) | 32 | (41.6%) | |

continuous variable: Mann-Whitney
categorical variable: Chi-square
n/a: Chi-square calculations are only valid when all expected values are greater than 1.0 and at least 20% of the expected vaules are greater than 5. These conditions have not been met, and thus the chi-square calculations are not valid.
*For CaP/AgCaP group, GS8 consists of 3 GS4 + 4 and 1 GS5 + 3
#For CaP/AgCaP group, GS9 consists of 8 GS4 + 5 and 2 GS5 + 4

### 1.5 US Patient Cohort and Trial Parameters

A prospective clinical trial was designed to collect a representative contemporary patient population from the United States of America. This meant that the study had representative frequencies of different ethnic groups in the USA and also reflected the contemporary prevalence of either no cancer, non-aggressive prostate cancer or aggressive prostate cancer. All patients who were recruited to the trial presented on the basis of an elevated age adjusted PSA and underwent biopsy at their local clinical site. Serum and plasma samples were collected together with a blood sample for standardized PSA test (performed in a central lab on an Abbott Architect machine). In addition to the biopsy assessment at the local site, a central biopsy review was performed by a single pathologist. The central PSA value and central biopsy classification were used for model development. The full details of the trial are described in Shore et al, Urologic Oncology Apr Volume 38, Issue 8, August 2020, Pages 683.el-683.e10.

The prospective non-randomized case-control study was designed having primary and secondary endpoints:

Primary endpoint: detection of prostate cancer vs non-prostate cancer patients.

Secondary endpoint: differentiation of aggressive (defined as Gleason ≥3+4) vs non-aggressive (defined as Gleason 3+3) prostate cancer.

The study was conducted in 12 US research centers and accrued a total of 384 subjects:
Arm 1 (Healthy Normal): 52 patients
Arm 2 (Prostate Biopsy): 332 (100%) patients
   Cohort A: 148 patients (45%), no cancer
   Cohort B: 64 patients (19%), GS=6, CaP
   Cohort C: 120 patients (36%), GS≥7 (≥3+4), CaP

Serum and plasma samples were collected, and standardized PSA test and centralized pathology were reviewed (both Gleason Score and Epstein scores).

Inclusion criteria were as follows:

### ARM 1: Healthy Normal (HN)

Subjects 50 years or older
Low PSA (performed at most 12 months prior) with low PSA defined as: <1.5 ng/mL between ages 50 and 60, <3 ng/mL above age 60
Signed informed consent

### ARM 2: Prostate Biopsy

Subjects 40 years or older
All subjects who were referred for or had undergone either a de novo or a repeat prostate biopsy for high PSA where high PSA was defined as: ≥1 ng/ml between ages 40 and 49, ≥2 ng/mL between ages 50 and 60, ≥3 ng/mL for age 60 and above
Signed informed consent.
   Exclusion criteria for ARM 1 were as follows:
1. Any subject with medical history of cancer except basal skin cancer or squamous skin cancer.
2. Any subject without PSA result or with PSA not within approved timeframe of at most 12 months.
3. Any subject who has had a DRE, ejaculated, or undertaken vigorous bike riding within 72 hours of blood draw.
4. Any subject with other lower urinary tract manipulation (defined as urological surgery, including prostate biopsy) in the previous 6 weeks from blood draw.
5. Any subject with benign prostatic hyperplasia as defined by the investigators review.
6. Any subject taking Saw Palmetto was excluded unless there was a minimum wash out of 30 days since last dose.
7. Any subject taking Androgen Deprivation Therapy.
8. Any subject taking Casodex was excluded unless there is a minimum wash out of 30 days since the last dose.
9. Any patient currently taking an experimental agent—placebo control or unknown agent.
10. Any subject taking 5 alpha reductase inhibitors is excluded unless there was a minimum 6 weeks washout since the last dose of finasteride and a minimum of 6 months wash out since the last dose of Dutasteride.
11. Any subject confirmed by the investigator to currently be suffering from prostatitis, proctodynia, or urinary tract infection.
   ARM 2 prostate cancer biopsy exclusion criteria were as follows:
1. Any subject with medical history of cancer other than prostate cancer except basal or squamous skin cancer.
2. Any subject without PSA result or with PSA not within approved timeframe of at most 12 months.
3. Any subject who has had a DRE, ejaculated, or undertaken vigorous bike riding within 72 hours of blood draw
4. Any subject with other lower urinary tract manipulation (defined as urological surgery, including prostate biopsy) in the previous 6 weeks from blood draw.
5. Any subject taking Saw Palmetto is excluded unless there was a minimum wash out of 30 days since the last dose.
6. Any subject taking Androgen Deprivation Therapy
7. Any subject taking Casodex was excluded unless there was a minimum wash out of 30 days since the last dose.
8. Any patient currently taking an experimental agent—placebo control or unknown agent.
9. Any subject taking 5 alpha reductase inhibitors is excluded unless there was a minimum of 6 weeks washout since the last dose of finasteride and a minimum of 6 months wash out since the last dose of Dutasteride.
10. Any subject confirmed by the investigator to currently be suffering from prostatitis, proctodynia or urinary tract infection.
   314 Arm 2 patient samples were used for this analysis. Of these, 302 had prostate volume data available.

Study patient characteristics are outlined in Table 2 below.

TABLE 2
patient characteristics for CUSP cohort

| | | | | | P | | | | | P | | | | | P |
|---|---|---|---|---|---|---|---|---|---|---|---|---|---|---|---|
| | | | | | value | | | | | Value | | | | | value |
| | | | | | (non- | | | | | (Not | | | | | (Non- |
| | | | | | CaP | | | | | AgCaP | | | | | AgCaP |
| | | | | | v | Not | | | | v | Non-Ag | | | | v |
| | Non-CaP | | CaP | | CaP) | AgCaP | | AgCaP | | AgCaP) | CaP | | AgCaP | | AgCaP) |
| Total | 139 | (44.3%) | 175 | (55.7%) | | 201 | (64.0%) | 113 | (36.0%) | | 62 | (35.4%) | 113 | (64.6%) | |
| Age | | | | | 0.15 | | | | | <0.01 | | | | | <0.01 |
| Mean (SD) | 63.76 | (7.75) | 65.26 | (8.21) | | 63.40 | (7.70) | 66.72 | (8.20) | | 62.60 | (7.60) | 66.72 | (8.20) | |
| Median | 64 | (40-82) | 65 | (45-85) | | 64 | (40-82) | 66 | (48-85) | | 62.5 | (45-79) | 66 | (48-85) | |
| (Range) | | | | | | | | | | | | | | | |
| >50 years, | 132 | (95.0%) | 169 | (96.6%) | | 190 | (94.5%) | 111 | (98.2%) | | 58 | (93.5%) | 111 | (98.2%) | |
| N(%) | | | | | | | | | | | | | | | |
| Prostate | | | | | <0.01 | | | | | <0.01 | | | | | 0.01 |
| Volume | | | | | | | | | | | | | | | |
| (cc) | | | | | | | | | | | | | | | |
| N measured | 137 | (98.6%) | 165 | (94.3%) | | 193 | (96.0%) | 109 | (96.5%) | | 56 | (90.3%) | 109 | (96.5%) | |
| (%) | | | | | | | | | | | | | | | |
| Biopsy | 81 | | 93 | | | 111 | | 63 | | | 30 | | 63 | | |
| Ultrasound | 50 | | 55 | | | 72 | | 33 | | | 22 | | 33 | | |
| MRI | 6 | | 17 | | | 10 | | 13 | | | 4 | | 13 | | |
| Mean (SD) | 64.19 | (34.95) | 42.42 | (19.47) | | 59.12 | (32.04) | 40.21 | (19.67) | | 46.71 | (18.51) | 40.21 | (19.67 | |
| Median | 51.80 (19.60- | | 38.00 (12.70- | | | 49.00 (18.10- | | 37.00 (12.70- | | | 42.80 (18.10- | | 37.00 (12.70- | | |
| (Range) | 189.00) | | 121.30) | | | 189.00) | | 121.30) | | | 94.80) | | 121.30) | | |
| Enrolling | | | | | <0.01 | | | | | <0.01 | | | | | <0.01 |
| PSA | | | | | | | | | | | | | | | |
| (ng/ml) | | | | | | | | | | | | | | | |
| Mean (SD) | 6.55 | (3.92 | 8.12 | (6.08) | | 6.47 | (3.69) | 9.13 | (7.02) | | 6.30 | (3.14) | 9.13 | (7.02) | |
| Median | 5.42 (1.2- | | 6.30 (2.0- | | | 5.51 (1.2- | | 7.10 (3.65- | | | 5.63 (2.0- | | 7.10 (3.65- | | |
| (Range) | 31.73 | | 48.90 | | | 31.73) | | 48.90) | | | 18.63) | | 48.90) | | |
| Race, N (%) | | | | | 0.63 | | | | | 0.61 | | | | | 0.44 |
| White | 121 | (87.1%) | 157 | (89.7%) | | 175 | (87.1%) | 103 | (91.2%) | | 54 | (87.1%) | 103 | (91.2%) | |
| Black | 16 | (11.5%) | 17 | (9.7%) | | 23 | (11.9%) | 9 | (8.0%) | | 8 | (12.9%) | 9 | (8.0%) | |
| Other/Unknown | 2 | (1.4%) | 1 | (0.6%) | | 2 | (1.0%) | 1 | (0.9%) | | 0 | (0%) | 1 | (0.9%) | |
| Hispanic | | | | | 0.53 | | | | | 0.72 | | | | | 1.00 |
| Ethnicity, | | | | | | | | | | | | | | | |
| N(%) | | | | | | | | | | | | | | | |
| Yes | 15 | (10.8%) | 14 | (8.0%) | | 20 | (10.0%) | 9 | (8.0%) | | 5 | (8.1%) | 9 | (8.0%) | |
| No | 123 | (88.5%) | 158 | (90.3%) | | 179 | (89.1%) | 102 | (90.3%) | | 56 | (90.3%) | 102 | (90.3%) | |
| Unknown | 1 | (0.7%) | 3 | (1.7%) | | 2 | (1.0%) | 2 | (1.8%) | | 1 | (1.6%) | 2 | (1.8%) | |
| First Degree | | | | | 0.20 | | | | | 0.97 | | | | | 0.30 |
| Family | | | | | | | | | | | | | | | |
| History, N(%) | | | | | | | | | | | | | | | |
| Yes | 31 | (22.3%) | 55 | (31.4%) | | 55 | (27.4%) | 31 | (27.4%) | | 24 | (38.7%) | 31 | (27.4%) | |
| No | 94 | (67.6%) | 105 | (60.0%) | | 127 | (63.2%) | 72 | (63.7%) | | 33 | (53.2%) | 72 | (63.7%) | |
| Unknown | 14 | (10.1%) | 15 | (8.6%) | | 19 | (9.5%) | 10 | (8.8%) | | 5 | (8.1%) | 10 | (8.8%) | |
| DRE status | | | | | 0.04 | | | | | <0.01 | | | | | 0.05 |
| Normal | 106 | (76.3%) | 113 | (64.6%) | | 153 | (76.1%) | 66 | (58.4%) | | 47 | (75.8%) | 66 | (58.4%) | |
| Suspicious | 15 | (10.8%) | 36 | (20.6%) | | 22 | (10.9%) | 29 | (25.7%) | | 7 | (11.3%) | 29 | (25.7%) | |
| Unknown | 18 | (12.9%) | 26 | (14.9%) | | 26 | (12.9%) | 18 | (15.9%) | | 8 | (12.9%) | 18 | (15.9%) | |
| Gleason | | | | | | | | | | | | | | | |
| Score/Epstein, | | | | | | | | | | | | | | | |
| N(%) | | | | | | | | | | | | | | | |
| 6/1 | | | 62 | (35.4%) | | 62 | (30.8%) | | | | 62 | (100%) | | | |
| 7 (3 + 4)/2 | | | 56 | (32.0%) | | | | 56 | (49.6%) | | | | 56 | (49.6%) | |
| 7 (4 + 3)/3 | | | 41 | (23.4%) | | | | 41 | (36.3%) | | | | 41 | (36.3%) | |
| 8/4* | | | 5 | (2.9%) | | | | 5 | (4.4%) | | | | 5 | (4.4%) | |
| 9/5# | | | 11 | (6.3%) | | | | 11 | (9.7%) | | | | 11 | (9.7%) | |

continuous variable: Mann-Whitney
categorical variable: Chi-square
n/a: Chi-square calculations are only valid when all expected values are greater than 1.0 and at least 20% of the expected values are greater than 5. These conditions have not been met, and thus chi-square calculations are not valid.
*For CaP/AgCaP, GS8 group consists of 1 GS3 + 5, 4 GS4 + 4
#For CaP/AgCaP, GS9 group consists of 9 GS4 + 5 and 2GS5 + 4

### 1.4 Sample Collection

For the Australian sample set, blood samples were either processed directly in to serum and measured or frozen at −80° C. prior to measurement. For the US sample set, whole blood samples taken from patients were stored at 4° C. and subjected to centrifugation within 2 hours of collection to separate serum components, which were stored at −20° C. Samples were shipped from the collection sites then thawed, aliquoted, and stored at −80° C.

### 1.5 Sample Measurement

Serum samples were measured at either DHM Laboratories (Macquarie Park, Sydney Australia) using Abbott Architect (total PSA and free PSA) or Abbott Alinity analyzers (HE4), or at Minomic Inc laboratories (Gaithersberg, USA) using a Roche Cobas analyzer (PSA, free PSA, HE4) according to the manufacturer's instructions.

### 1.6 Model Development and Results

192 samples from the Macquarie Australian study had complete data for evaluation and were measured using the Abbott analyzers. MRI data and prostate biopsy results were taken from the patient's medical records. DRE status was obtained from the medical record. For model development, missing DRE data was treated as non-suspicious. Patients missing PIRADs values were assigned a value of 0 and added to the PIRADs 1-3 groups for analysis.

314 samples from Arm the CUSP US study were measured by the Abbott analyzers (302 of which had prostate volume). 300 samples with available prostate volume were measured using the Roche analyzer. These samples all had centrally scored biopsy results.

For each cohort, a combined database was generated linking the clinical and demographic factors to the analyte sample values. Out of range and extrapolated data were set to either top or bottom values of standard curve for each analyte.
1. PSA, % free PSA, free PSA and HE4 analyte values were log transformed to achieve normal distribution for model development.
2. No CaP: was defined as patients without prostate cancer (no cancer on biopsy)
3. CaP: patients with prostate cancer (GS≥3+3).
4. NonAgCaP: patients with non-aggressive prostate cancer defined as Gleason Score equal to 3+3.
5. NOT AgCaP=No CaP+NonAgCaP
6. AgCaP: patients with aggressive prostate cancer defined as Gleason Score equal to 3+4 or higher.

312 samples from the CUSP trial had been measured on both the Abbott Architect and Alinity platforms as well as the Roche Cobas platforms. A between platform correlation analysis was performed (Table 3).

TABLE 3
Correlations between Cobas results versus
Architect results for the 3 tumor markers

| Marker | tPSA | fPSA | WFDC2(HE4) |
|---|---|---|---|
| Slope | 0.9 (95% CI, | 1.21(95% CI, | 0.76(95%, 0.73- |
| | 0.88-0.92) | 1.18-1.25) | 0.79) |
| Intercept | −0.14 | −0.05 | −3.56 |
| Correlation | 0.987 | 0.952 | 0.956 |
| Coefficient R | | | |

The results in Table 3 indicate that there is good correlation between the Abbott and Roche platforms (correlation co-efficient R>0.9 for all three markers), however the slope of the correlation line is not 1, indicating that the concentration values obtained for each sample will not be the same between the different platforms. This means that a model developed on one platform may not perform equivalently when the samples are measured on a different platform and that different models may need to be developed for the different platforms.

To develop multi-variate models, the following steps were used:

1. Imported the combined data set into the R computer program (R Core Team (2017). R: A language and environment for statistical computing. R Foundation for Statistical Computing, Vienna, Austria. ISBN 3-900051-07-0, URL http://www.R-project.org/) loaded with the BMA (Raferty et al, 2018 BMA: Bayseian Model Averaging R Package 3.18.8), VSURF (Genuer, R. and Poggi, J. M. and Tuleau-Malot, C. (2010), Variable selection using random forests, Pattern Recognition Letters 31(14), 2225-2236 and Genuer, R. and Poggi, J. M. and Tuleau-Malot, C. (2015), VSURF: An R Package for Variable Selection Using Random Forests, The R Journal 7(2):19-33), caret (Kuhn et al 2018, caret: Classification and Regression Training. R package version 6.0-79. https://CRAN.R-project.org/package=caret), ROCR (Robin et al, 2011, pROC: an open-source package for R and S+ to analyze and compare ROC curves”. BMC Bioinformatics, 7, 77. DOI: 10.1186/1471-2105-12-77), pROC (Sing T, Sander 0, Beerenwinkel N and Lengauer T (2005). “ROCR: visualizing classifier performance in R.” _Bioinformatics_, *21*(20), pp. 7881. <URL: http://rocr.bioinf.mpi-sb.mpg.de) stats packages.
2. Models were developed using combinations of WFDC2 (HE4), PSA, Free PSA, % Free PSA, DRE, PV, Family History and PIRADs scores from the following data sets:
(a) the Macquarie data set measured on the Abbott analyzers
(b) the CUSP data set measured on the Abbott Analyzers
(c) the combined Macquarie and CUSP data sets measured on the Abbott analyzers
(d) the CUSP data set measured on the Roche analyzer.
   3. Models were developed either on the entire available data set, or on a subset thereof

Model development and ROC analyses (aggressive prostate cancer versus non-aggressive and no prostate cancer) were performed for PSA (Figure One), Prostate Volume (Figure Two), % free PSA (Figure Three), Free PSA (Figure Four), WFDC2 (HE4) (Figure Five), PIRADs (Figure Six) Age (Figure Seven) and DRE (Figure Eight). The performance of the different models for the individual components is shown in Table 4.

TABLE 4
Performance of individual components in differentiating aggressive cancer
from non-aggressive and no cancer patients in the MQ population.

| | | | | | | | Specificity | Specificity | Specificity |
|---|---|---|---|---|---|---|---|---|---|
| | | Population | # | | Model | | at 94% | at 92% | at 90% |
| Model | Componenets | Developed | patients | Platform | Type | AUC | sensitivity | sensitivity | sensitivity |
| 1 | PSA | MQ192 | 192 | Abbott | Standard | 0.58 (0.5-0.67) | 13 | 17 | 19 |
| 2 | Prostate | MQ192 | 192 | Abbott | Standard | 0.69 (0.62-0.77) | 31 | 35 | 35 |
| | volume (PV) | | | | | | | | |
| 3 | % free PSA | MQ192 | 192 | Abbott | Standard | 0.68 (0.6-0.76) | 22 | 22 | 25 |
| 4 | Free PSA | MQ192 | 192 | Abbott | Standard | 0.54 (0.46-0.63) | 9 | 13 | 19 |
| 5 | WFDC2 (HE4) | MQ192 | 192 | Abbott | Standard | 0.54 (0.46-0.63) | 10 | 16 | 17 |
| 6 | PIRADs* | MQ192 | 192 | Abbott | Standard | 0.65 (0.57-0.72) | PIRADS ≥3 (96%/20%), ≥4 (84%/32%), 5 | | |
| | | | | | | | (42%/80% | | |
| 7 | Age | MQ192 | 192 | Abbott | Standard | 0.65 (0.57-0.73) | 14 | 17 | 18 |

| | | | | | | | |
|---|---|---|---|---|---|---|---|
| 8 | DRE* | MQ192 | 192 | Abbott | Standard | 0.54 (0.48-0.61) | Suspicious DRE: sens 29%/Spec 80% |

*means that ROC curve shape did not allow determination of specificity at fixed 94%, 92% or 90% sensitivity

The goal of the model development was to improve on currently available clinical tests such as PSA, DRE, % free PSA and/or PIRADs score in the ability to accurately predict the presence of aggressive prostate cancer.

For each Logistic regression model, one or more of PSA, % free PSA, Free PSA and HE4 values were obtained and log transformed. The transformed values were multiplied by their respective log odds ratio co-efficient. If an abnormal/suspicious DRE status was obtained, it was multiplied by its log odds ratio co-efficient. If PIRADs status was obtained, it was multiplied by its log odds ratio co-efficient. If Age was obtained, it was multiplied by its log odds ratio co-efficient. The products were summed to generate a Logit(P) value which was then used in the following equation to generate a probability score P.

The General equation is:

$$\begin{matrix}Logit ( P ) = Log ( P / 1 - P ) \\ = intercept + {\sum }_{i = 1}^{N} ( {coefficient}_{i} \times transformed ( {variable}_{i} ) \\ P = \frac{exp ( Logit ( P ) )}{1 + exp ( Logit ( P ) )}\end{matrix} \qquad ( i )$$

wherein:
P is probability of that the test subject has aggressive prostate cancer,
the coefficientᵢ is the natural log of the odds ratio of the variable,
the transformed variableᵢ is the natural log of the variableᵢ value; or

$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( ii )$$

wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable
the variableⱼ is the numerical value of the variableⱼ; or

$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( iii )$$

wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable,
the variableⱼ is the numerical value of the variableⱼ,
variableⱼ can be one or more of DRE value, Age, or PIRADS score,
if variableⱼ is DRE, a DRE value of 1 indicates an abnormal DRE status and a DRE value of 0 indicates a normal DRE status,
if variableⱼ is PIRADs score, the PIRADS score is 1, 2, 3, 4 or 5, and if a PIRADS score is not available, variableⱼ is 0, and
if variableⱼ is Age, the Age is in years.

The contribution of additional analytes to the performance of different models is shown in Table 5.

TABLE 5
Comparison of models developed using 1-7 markers in the MQ population

| | | | | | | | Specificity | Specificity | Specificity |
|---|---|---|---|---|---|---|---|---|---|
| | | Population | # | | Model | | at 94% | at 92% | at 90% |
| Model | Components | Developed | patients | Platform | type | AUC | Sensitivity | Sensitivity | Sensitivity |
| 9 | WFDC2 (HE4), PSA, % free PSA | MQ192 | 192 | Abbott | Standard | 0.68 (0.6-0.75) | 17 | 18 | 20 |
| 10 | WFDC2 (HE4), PSA, % free PSA + | MQ192 | 192 | Abbott | Standard | 0.72 (0.65-0.8) | 27 | 34 | 37 |
| | PV | | | | | | | | |
| 11 | WFDC2 (HE4), PSA, % free PSA + | MQ192 | 192 | Abbott | Standard | 0.79 (0.73-0.86) | 37 | 39 | 44 |
| | PV + Age | | | | | | | | |
| 12 | WFDC2 (HE4), PSA, % free PSA + | MQ192 | 192 | Abbott | Standard | 0.75 (0.68-0.82) | 33 | 35 | 42 |
| | PV + PIRADs | | | | | | | | |
| 13 | WFDC2 (HE4), PSA, % free PSA + | MQ192 | 192 | Abbott | Standard | 0.8 (0.74-0.87) | 35 | 39 | 44 |
| | PV + PIRADs + Age | | | | | | | | |
| 14 | WFDC2 (HE4), PSA, % free PSA + | MQ192 | 192 | Abbott | Standard | 0.8 (0.74-0.87) | 36 | 40 | 43 |
| | PV + PIRADs + Age + DRE | | | | | | | | |
| 15 | WFDC2 (HE4), PSA, % free PSA + | MQ192 | 192 | Abbott | Standard | 0.71 (0.64-0.79) | 31 | 33 | 34 |
| | PIRADs | | | | | | | | |
| 16 | WFDC2 (HE4), PSA, % free PSA + | MQ192 | 192 | Abbott | Standard | 0.73 (0.66-0.81) | 16 | 28 | 30 |
| | Age | | | | | | | | |
| 17 | WFDC2 (HE4), PSA, % free PSA + | MQ192 | 192 | Abbott | Standard | 0.68 (0.6-0.76) | 19 | 23 | 25 |
| | DRE | | | | | | | | |
| 18 | WFDC2 (HE4), PSA, % free PSA + | MQ192 | 192 | Abbott | Standard | 0.73 (0.66-0.81) | 19 | 26 | 26 |
| | Age + DRE | | | | | | | | |
| 19 | WFDC2 (HE4), PSA, % free PSA + | MQ192 | 192 | Abbott | Standard | 0.75 (0.68-0.83) | 23 | 28 | 30 |
| | Age + FH | | | | | | | | |
| 20 | WFDC2 (HE4), PSA, % free PSA + | MQ192 | 192 | Abbott | Standard | 0.75 (0.68-0.83) | 23 | 25 | 25 |
| | Age + DRE + FH | | | | | | | | |
| 21 | WFDC2 (HE4), % free PSA + PV | MQ192 | 192 | Abbott | Standard | 0.72 (0.65-0.79) | 30 | 37 | 43 |
| 22 | WFDC2 (HE4), % free PSA + PV + | MQ192 | 192 | Abbott | Standard | 0.78 (0.72-0.85) | 25 | 30 | 44 |
| | Age | | | | | | | | |
| 23 | WFDC2 (HE4), % free PSA + PV + | MQ192 | 192 | Abbott | Standard | 0.74 (0.67-0.81) | 30 | 34 | 44 |
| | PIRADs | | | | | | | | |
| 24 | WFDC2 (HE4), % free PSA + PV + | MQ192 | 192 | Abbott | Standard | 0.8 (0.73-0.86) | 28 | 30 | 41 |
| | PIRADs + Age + DRE | | | | | | | | |
| 25 | WFDC2 (HE4), % free PSA + PV + | MQ192 | 192 | Abbott | Standard | 0.8 (0.73-0.86) | 28 | 30 | 41 |
| | PIRADs + Age | | | | | | | | |
| 26 | WFDC2 (HE4), % free PSA + PIRADs | MQ192 | 192 | Abbott | Standard | 0.71 (0.64-0.79) | 26 | 28 | 31 |
| 27 | WFDC2 (HE4), % free PSA + Age | MQ192 | 192 | Abbott | Standard | 0.73 (0.66-0.81) | 14 | 28 | 30 |
| 28 | WFDC2 (HE4), PSA, free PSA | MQ192 | 192 | Abbott | Standard | 0.68 (0.6-0.75) | 17 | 18 | 20 |
| 29 | WFDC2 (HE4), PSA, free PSA + PV | MQ192 | 192 | Abbott | Standard | 0.72 (0.65-0.8) | 27 | 34 | 37 |
| 30 | WFDC2 (HE4), PSA, free PSA + | MQ192 | 192 | Abbott | Standard | 0.79 (0.73-0.86) | 37 | 39 | 44 |
| | PV + Age | | | | | | | | |
| 31 | WFDC2 (HE4), PSA, free PSA + | MQ192 | 192 | Abbott | Standard | 0.75 (0.68-0.82) | 33 | 35 | 41 |
| | PV + PIRADs | | | | | | | | |
| 32 | WFDC2 (HE4), PSA, free PSA + | MQ192 | 192 | Abbott | Standard | 0.8 (0.74-0.87) | 35 | 39 | 44 |
| | PV + PIRADs + Age | | | | | | | | |
| 33 | WFDC2 (HE4), PSA, free PSA + | MQ192 | 192 | Abbott | Standard | 0.71 (0.64-0.79) | 31 | 34 | 34 |
| | PIRADs | | | | | | | | |
| 34 | WFDC2 (HE4), PSA, free PSA + Age | MQ192 | 192 | Abbott | Standard | 0.73 (0.66-0.81) | 16 | 28 | 30 |

Of note, the base model 9 of WFDC2(HE4), PSA and % free PSA had relatively low specificity at sensitivities of 94%, 92% and 90%. Inclusion of additional individual variables into the model such as PV, Age, PIRADs score and to a lesser extent, Age, all increased the model AUC and specificities at the fixed sensitivities in this population (with the exception of Age at 94% sensitivity). Further improvements in specificity were observed when more than one additional variable was added to the base model—e.g. adding PV and Age (Model 11) resulted in increases of specificity from the base model from 17 to 37% (94% sensitivity), 18 to 39% (92% sensitivity) and 20 to 44% (90% sensitivity).

Models without Total PSA.

PSA was not significantly different between the patients with aggressive cancer and those without (p=0.13, Table 1). Therefore, models were developed in which total PSA was not included as a component, but which used WFDC2(HE4) and % free PSA as a base combination and added additional factors to the model. As can be seen from comparing models 10 and 21, a model in which total PSA is omitted resulted in higher specificities at sensitivities of 94, 92 and 90% sensitivities. However, comparison of models 11 and 22 indicated that omission of PSA did not always result in higher specificities and fixed sensitivities.

Models with Free PSA (Instead of % Free PSA)

Free PSA is the analyte measured, while % free PSA is a derived value that incorporates total PSA. Comparison of Models 9 and 28, 10 and 29, 11 and 30, indicated that equivalent model performance could also be achieved by using the Free PSA value rather than the % free PSA value.

### Preferred Models for Pre-MRI

As shown in the workflow diagram for pre-MRI in Sections 1.1 and 1.3, a preferred model would incorporate data and analyte values that do not require an MRI. A preferred model would give high performance, have the minimal number of components and use data that was easily collected. Collection of family history is often dependent on recall of the subject and is not always collected. Collection of the patient DRE status was not recorded in 44 of 192 (23%) subjects and is often not performed in Australia due to patient preference. In contrast, Age is collected for every blood sample and therefore is a reliable marker.

### Development of Standard Logistic Regression Models in the PIRADS 1-3 Population

Due to the current MRI workflow and biopsy practice, there is significant unmet need in identifying aggressive cancer in patients with PIRADs scores of 1-3. There were 41 patients with PIRADs scores of 1-3 in the MQ population and 8 patients who had prostate volume information available but no PIRADs score reported. These 8 patients were grouped in to the PIRADs 1-3 population for the purposes of this analysis.

Logistic regression models were developed using data from these 49 patients and a selection of marker combinations identified from the 192 patient population. Due to the small number of patients, sensitivity/specificity data could not be reported at the 94%, 92% and 90% sensitivity cutpoints, but was instead reported at 92%, 83% and 75% sensitivities. The performance of the different marker combinations is shown in Table 6.

TABLE 6
Comparison of models developed using 1-5 markers in the MQ PIRADs 1-3 population

| | | | | | | | Specificity | Specificity | Specificity |
|---|---|---|---|---|---|---|---|---|---|
| | | Population | # | | Model | | at 94% | at 92% | at 90% |
| Model | Components | Developed | patients | Platform | type | AUC | Sensitivity | Sensitivity | Sensitivity |
| 35 | PSA | MQ49 | 49 | Abbott | Standard | 0.53 (0.36-0.7) | 92/30 | 83/35 | 75/35 |
| 36 | WFDC2 (HE4), PSA, % free PSA | MQ49 | 49 | Abbott | Standard | 0.68 (0.51-0.85) | 92/43 | 83/43 | 75/43 |
| 37 | WFDC2 (HE4), PSA, % free PSA + | MQ49 | 49 | Abbott | Standard | 0.77 (0.62-0.92) | 92/51 | 83/57 | 75/65 |
| | PV | | | | | | | | |
| 38 | WFDC2 (HE4), PSA, % free PSA + | MQ49 | 49 | Abbott | Standard | 0.79 (0.64-0.94) | 92/54 | 83/59 | 75/65 |
| | PV + Age | | | | | | | | |
| 39 | WFDC2 (HE4), PSA, free PSA | MQ49 | 49 | Abbott | Standard | 0.68 (0.51-0.85) | 92/43 | 83/43 | 75/43 |
| 40 | WFDC2 (HE4), PSA, free PSA + | MQ49 | 49 | Abbott | Standard | 0.77 (0.62-0.92) | 92/51 | 83/57 | 75/65 |
| | PV | | | | | | | | |
| 41 | WFDC2 (HE4), PSA, free PSA + | MQ49 | 49 | Abbott | Standard | 0.79 (0.64-0.94) | 92/54 | 83/59 | 75/65 |
| | PV + Age | | | | | | | | |

Similar to the results observed in the whole population, total PSA was relatively poor at identifying aggressive prostate cancer in this subset (AUC 0.53). The base combination of WFDC2(HE4), total PSA, % free PSA improved the AUC to 0.68 and increased specificity at each sensitivity cutpoint. Inclusion of PV further improved the AUC and specificity, and inclusion of PV and age further improved the AUC and specificity (with the exception of 75% sensitivity cutpoint). Performance at 92% sensitivity for the WFDC2(HE4), PSA, % free PSA and PV, and the WFDC2(HE4), PSA, % free PSA, PV and Age models was higher than that observed in the models developed on the whole population (51% specificity vs 34% and 54% specificity vs 39% specificity), albeit that the sample size is small. Replacement of % free PSA with Free PSA in the models did not change the model performance for AUC, sensitivity or specificity.

These results suggest that the marker combinations identified as having utility in the whole population can also show utility in the clinically important PIRADs 1-3 subset.

### Development of Standard Logistic Regression Models in the CUSP US Population

The ability of the same marker combinations identified above to differentiate aggressive prostate cancer was assessed in the US population (Table 7). Given the differences observed for the same samples measured on different analytical platforms (Table 3), a comparison was also made between the performance of models developed using samples measured on the Abbott or the Roche analytical platforms (Table 7). Prostate Volume was available for 302 of the CUSP samples, and sufficient samples were available to measure 302 (Abbott) or 300 (Roche) samples.

A higher performance (AUC 0.81 vs 0.68, 94% sensitivity/41% specificity vs 94% sensitivity/17% specificity) was observed for the core WFDC2(HE4), PSA, % free PSA model in the CUSP sample set compared to the MQ 192 samples when all were measured on the Abbott Architect platform (Model 44 vs Model 9). Similar increases in Algorithm performance were observed in the CUSP (Abbott) algorithms when PV (Model 45), Age (Model 47), or PV and Age (Model 46) were incorporated into the models, with AUC increasing from 0.81 to 0.84, 0.82 or 0.86 respectively, while specificity at 94% increased from 41% to 48%, 55% and 57% respectively. The combination of WFDC2(HE4), PSA, % free PSA, PV and Age (Model 46) had the highest performance, consistent with what was observed in the MQ 192 sample set. Measurement of the CUSP samples on the Roche platform generated model performance that was similar to the Abbott date for the AUC (Models 48-51). For both platforms, the combination of WFDC2(HE4), PSA, % free PSA, PV and Age gave the highest AUC and the highest specificity at 94% and 92% sensitivities. For both Abbott and Roche data sets, use of % free PSA rather than Free PSA resulted in models of equivalent AUC with equivalent sensitivity/specificities.

TABLE 7
Comparison of models developed using 1-5 markers in the CUSP US population

| | | | | | | | Specificity | Specificity | Specificity |
|---|---|---|---|---|---|---|---|---|---|
| | | Population | # | | Model | | at 94% | at 92% | at 90% |
| Model | Components | Developed | patients | Platform | type | AUC | Sensitivity | Sensitivity | Sensitivity |
| 42 | PSA | MICheck 302 | 302 | Abbott | Standard | 0.73 (0.68-0.79) | 41 | 42 | 47 |
| 43 | PSA | MICheck 300 | 300 | Roche | Standard | 0.72 (0.66-0.78) | 32 | 35 | 37 |
| 44 | WFDC2 (HE4), PSA, % | MICheck 302 | 302 | Abbott | Standard | 0.81 (0.76-0.86) | 41 | 52 | 52 |
| | free PSA | | | | | | | | |
| 45 | WFDC2 (HE4), PSA, % | MICheck 302 | 302 | Abbott | Standard | 0.84 (0.79-0.89) | 48 | 49 | 52 |
| | free PSA + PV | | | | | | | | |
| 46 | WFDC2 (HE4), PSA, % | MICheck 302 | 302 | Abbott | Standard | 0.86 (0.82-0.9) | 57 | 60 | 62 |
| | free PSA + PV + Age | | | | | | | | |
| 47 | WFDC2 (HE4), PSA, % | MICheck 302 | 302 | Abbott | Standard | 0.82 (0.78-0.87) | 55 | 55 | 56 |
| | free PSA + Age | | | | | | | | |
| 48 | WFDC2 (HE4), PSA, % | MICheck 300 | 300 | Roche | Standard | 0.81 (0.75-0.86) | 32 | 33 | 44 |
| | free PSA | | | | | | | | |
| 49 | WFDC2 (HE4), PSA, % | MICheck 300 | 300 | Roche | Standard | 0.84 (0.8-0.89) | 43 | 46 | 59 |
| | free PSA + PV | | | | | | | | |
| 50 | WFDC2 (HE4), PSA, % | MICheck 300 | 300 | Roche | Standard | 0.85 (0.81-0.9) | 48 | 58 | 63 |
| | free PSA + PV + Age | | | | | | | | |
| 51 | WFDC2 (HE4), PSA, % | MICheck 300 | 300 | Roche | Standard | 0.81 (0.77-0.86) | 43 | 46 | 48 |
| | free PSA + Age | | | | | | | | |
| 52 | WFDC2 (HE4), PSA, free | MICheck 302 | 302 | Abbott | Standard | 0.81 (0.76-0.86) | 41 | 52 | 52 |
| | PSA | | | | | | | | |
| 53 | WFDC2 (HE4), PSA, free | MICheck 302 | 302 | Abbott | Standard | 0.84 (0.79-0.89) | 48 | 48 | 52 |
| | PSA + PV | | | | | | | | |
| 54 | WFDC2 (HE4), PSA, free | MICheck 302 | 302 | Abbott | Standard | 0.86 (0.82-0.9) | 57 | 60 | 62 |
| | PSA + PV + Age | | | | | | | | |
| 55 | WFDC2 (HE4), PSA, free | MICheck 302 | 302 | Abbott | Standard | 0.82 (0.78-0.87) | 55 | 55 | 56 |
| | PSA + Age | | | | | | | | |
| 56 | WFDC2 (HE4), PSA, free | MICheck 300 | 300 | Roche | Standard | 0.81 (0.75-0.86) | 32 | 33 | 44 |
| | PSA | | | | | | | | |
| 57 | WFDC2 (HE4), PSA, free | MICheck 300 | 300 | Roche | Standard | 0.84 (0.8-0.89) | 43 | 46 | 59 |
| | PSA + PV | | | | | | | | |
| 58 | WFDC2 (HE4), PSA, free | MICheck 300 | 300 | Roche | Standard | 0.85 (0.81-0.9) | 48 | 58 | 63 |
| | PSA + PV + Age | | | | | | | | |
| 59 | WFDC2 (HE4), PSA, free | MICheck 300 | 300 | Roche | Standard | 0.81 (0.77-0.86) | 43 | 46 | 48 |
| | PSA + Age | | | | | | | | |

### Development of Standard Logistic Regression Models Using the Combined CUSP and MQ Data Sets

The CUSP and MQ data sets measured on the Abbott analyzers were combined into a single database to determine the differences in performance between data sets, and whether it was possible to develop a model that would perform with high sensitivity and specificity across both data sets. The combination of WFDC2(HE4), PSA, % free PSA and Age was identified as a preferred variable combination in prior analyses. A model using these analytes was developed on the 506 combined data set (314 CUSP+192 MQ samples, all measured on the Abbott analyzers (Table 8). Model 60 demonstrates that the performance of the pre-MRI preferred variable combination of WDFC2(HE4), PSA, % free PSA and Age is lower in the combined population compared to the CUSP population (Model 47) (AUC 0.78 vs. 0.82, Model 60 vs 47), but higher than in the MQ population (AUC 0.78 vs 0.73, Model 60 vs 16). Applying the 506 combined model 60 to the MQ 192 population produced a slightly lower AUC (0.72 vs 0.73) but higher specificity at defined sensitivities (94% sensitivity 29% specificity vs 16% sensitivity, 92% sensitivity 30% vs 28% specificity, 90% sensitivity 32% specificity vs 30% specificity) than Model 16 developed on the 192 patient population. In contrast applying the 506 combined model to the CUSP 314 population (Model 62) produced a higher AUC (0.82 vs 0.78) and higher specificity at defined sensitivities (94% sensitivity 48% specificity vs 40% sensitivity, 92% sensitivity 52% vs 43% specificity, 90% sensitivity 57% specificity vs 46% specificity) than Model 60 applied to the combined patient population.

TABLE 8
Performance of models developed on the combined CUSP + MQ databases on the combined and individual sample sets

| | | | | | | | Specificity | Specificity | Specificity |
|---|---|---|---|---|---|---|---|---|---|
| | | Population | # | | Model | | at 94% | at 92% | at 90% |
| Model | Components | Developed | patients | Platform | type | AUC | Sensitivity | Sensitivity | Sensitivity |
| 60 | WFDC2 (HE4), PSA, % free | MQ192 + | 506 | Abbott | Standard | 0.78 (0.74-0.82) | 40 | 43 | 46 |
| | PSA + Age | MICheck 314 | | | | | | | |
| 61 | WFDC2 (HE4), PSA, % free | MQ192 + | 192 | Abbott | Standard | 0.72 (0.64-0.79) | 29 | 30 | 32 |
| | PSA + Age | MICheck 314 | | | | | | | |
| 62 | WFDC2 (HE4), PSA, % free | MQ192 + | 314 | Abbott | Standard | 0.82 (0.77-0.86) | 48 | 52 | 57 |
| | PSA + Age | MICheck 314 | | | | | | | |
| 63 | WFDC2 (HE4), PSA, % free | MQ192 + | 494 | Abbott | Standard | 0.79 (0.75-0.83) | 33 | 35 | 37 |
| | PSA + PV | MICheck 302 | | | | | | | |
| 64 | WFDC2 (HE4), PSA, % free | MQ192 + | 494 | Abbott | Standard | 0.82 (0.79-0.86) | 44 | 48 | 56 |
| | PSA + PV + Age | MICheck 302 | | | | | | | |
| 65 | WFDC2 (HE4), PSA, % free | MQ192 | 192 | Abbott | Standard | 0.78 (0.71-0.85) | 28 | 42 | 46 |
| | PSA + PV + Age | MICheck 302 | | | | | | | |
| 66 | WFDC2 (HE4), PSA, % free | MQ192 + | 302 | Abbott | Standard | 0.85 (0.81-0.9) | 50 | 57 | 60 |
| | PSA + PV + Age | MICheck 302 | | | | | | | |
| 67 | WFDC2 (HE4), PSA, free | MQ192 + | 506 | Abbott | Standard | 0.75 (0.71-0.79) | 27 | 36 | 37 |
| | PSA | MICheck 314 | | | | | | | |
| 68 | WFDC2 (HE4), PSA, free | MQ192 + | 494 | Abbott | Standard | 0.79 (0.75-0.83) | 33 | 35 | 37 |
| | PSA + PV | MICheck 302 | | | | | | | |
| 69 | WFDC2 (HE4), PSA, free | MQ192 + | 494 | Abbott | Standard | 0.82 (0.79-0.86) | 44 | 48 | 56 |
| | PSA + PV + Age | MICheck 302 | | | | | | | |
| 70 | WFDC2 (HE4), PSA, free | MQ192 + | 506 | Abbott | Standard | 0.78 (0.74-0.82) | 40 | 43 | 46 |
| | PSA + Age | MICheck 314 | | | | | | | |

### 1.7 Detailed Analysis of Preferred Models

Algorithms for Use Prior to mpMRI

The base model of WFDC2(HE4), PSA, % free PSA (Model 9)

### Standard Logistic Regression Developed on the MQ192 Population Only

Model developed to differentiate AgCaP vs NOTAgCaP
Data for model development: all patients (77 AgCaP, 115 NOTAgCaP)
Data for performance report: all patients (77 AgCaP, 115 NOTAgCaP)
Method: Standard Multivariable Logistic Regression
AUC is 0.68 (0.6-0.75), ROC curve is shown in Figure Nine

TABLE 9
Variables, transformations and Log Odds ratio for Model 9

| Variable | Transformation | Log Odd ratio |
|---|---|---|
| (Intercept) | | 1.4370270334 |
| Total.PSA | Log | 0.3006268516 |
| % Free.PSA | Log | −1.2233700659 |
| HE4 (pmol/L) | Log | 0.3106202049 |

TABLE 10
Sensitivity, Specificity and Accuracy
for Model 9 at different Thresholds

| | Sensitivity | Specificity | Accuracy |
|---|---|---|---|
| Threshold | (%) | (%) | (%) |
| 0.2448329076 | 90 | 20 | 47.9 |
| 0.2399910519 | 91 | 19 | 47.9 |
| 0.2314162824 | 92 | 18 | 47.9 |
| 0.2274226511 | 94 | 17 | 47.4 |
| 0.2216996506 | 95 | 17 | 47.9 |

A preferred model for pre-MRI assessment was selected as WFDC2(HE4), PSA, % free PSA and Age (Model 16).

### Standard Logistic Regression Developed on the MQ192 Population Only

Model developed to differentiate AgCaP vs NOTAgCaP
Data for model development: all patients (77 AgCaP, 115 NOTAgCaP)
Data for performance report: all patients (77 AgCaP, 115 NOTAgCaP)
Method: Standard Multivariable Logistic Regression
AUC is 0.73 (0.66-0.81), ROC curve is shown in Figure Ten

TABLE 11
Variables, transformations and Log Odds ratio for Model 16

| Variable | Transformation | Log Odd ratio |
|---|---|---|
| (Intercept) | | −0.46982517441 |
| Total.PSA | Log | 0.06876585881 |
| % Free.PSA | Log | −1.41956083126 |
| HE4 (pmol/L) | Log | −0.55891998656 |
| Age | | 0.09408487434 |

TABLE 12
Sensitivity, Specificity and Accuracy
for Model 16 at different Thresholds

| | Sensitivity | Specificity | Accuracy |
|---|---|---|---|
| threshold | (%) | (%) | (%) |
| 0.2367708800 | 90 | 30 | 53.6 |
| 0.2256961581 | 91 | 28 | 53.1 |
| 0.2245890687 | 92 | 28 | 53.6 |
| 0.1569765942 | 94 | 16 | 46.9 |
| 0.1482610342 | 95 | 15 | 46.9 |

### Development of Algorithms for Use Post-MRI

A preferred model for post-MRI assessment was selected as WFDC2(HE4), PSA, % free PSA, Age, PV (Model 11).

### Standard Logistic Regression Developed on the MQ192 Population Only

Model developed to differentiate AgCaP vs. NOTAgCaP
Data for model development: all patients (77 AgCaP, 115 NOTAgCaP)
Data for performance report: all patients (77 AgCaP, 115 NOTAgCaP)
Method: Standard Multivariable Logistic Regression
AUC is 0.79 (0.73-0.86), ROC curve is shown in Figure Eleven

TABLE 13
Variables, transformations and Log Odds ratio for Model 11

| Variable | Transformation | Log Odd ratio |
|---|---|---|
| (Intercept) | | 4.2927367713 |
| Total.PSA | Log | 0.4911524327 |
| % Free.PSA | Log | −0.5616217330 |
| HE4 (pmol/L) | Log | −0.9199744925 |
| Age | | 0.1163616293 |
| PV | Log | −2.0630966782 |

TABLE 14
Sensitivity, Specificity and Accuracy
for Model 11 at different Thresholds

| | Sensitivity | Specificity | Accuracy |
|---|---|---|---|
| threshold | (%) | (%) | (%) |
| 0.21783011496 | 90 | 44 | 62.5 |
| 0.21320692301 | 91 | 44 | 63.0 |
| 0.18081398403 | 92 | 39 | 60.4 |
| 0.17329240055 | 94 | 37 | 59.9 |
| 0.14678314198 | 95 | 26 | 53.6 |

### Models Developed on 49 PIRADs 1-3 Patients

A model for post-MRI assessment using PSA, alone (Model 35).

### Standard Logistic Regression Developed on the MQ49 PIRADs 1-3 Population Only

Model developed to differentiate AgCaP vs NOTAgCaP
Data for model development: all patients (12 AgCaP, 37 NOTAgCaP)
Data for performance report: all patients (12 AgCaP, 37 NOTAgCaP)
Method: Standard Multivariable Logistic Regression
AUC is 0.53 (0.36-0.7), ROC curve is shown in Figure Twelve

A model for post-MRI assessment was selected as WFDC2(HE4), PSA, % free PSA, Age, PV (Model 38).

### Standard Logistic Regression Developed on the MQ49 PIRADs 1-3 Population Only

Model developed to differentiate AgCaP vs NOTAgCaP
Data for model development: all patients (12 AgCaP, 37 NOTAgCaP)
Data for performance report: all patients (12 AgCaP, 37 NOTAgCaP)
Method: Standard Multivariable Logistic Regression
AUC is 0.79 (0.64-0.94), ROC curve is shown in Figure Thirteen

TABLE 15
Variables, transformations and Log Odds ratio for Model 38

| Variable | Transformation | Log Odd ratio |
|---|---|---|
| (Intercept) | | 8.1137732787 |
| Total.PSA | Log | 0.7137236053 |
| % Free.PSA | Log | 0.1421348056 |
| HE4 (pmol/L) | Log | −1.6964303143 |
| PV | Log | −2.9903054802 |
| Age | | 0.1184545145 |

TABLE 16
Sensitivity, Specificity and Accuracy
for Model 38 at different Thresholds

| threshold | Sensitivity (%) | Specificity (%) | Accuracy (%) |
|---|---|---|---|
| 0.193935078103 | 75 | 65 | 67.3 |
| 0.164908674102 | 83 | 59 | 65.3 |
| 0.155461994714 | 92 | 54 | 63.3 |

### Standard Logistic Regression Developed on the CUSP 302 Population Using PSA the Abbott Architect Model 42

Model developed to differentiate AgCaP vs NOTAgCaP
Data for model development: all patients (109 AgCaP, 193 NOTAgCaP)
Data for performance report: all patients (109 AgCaP, 193 NOTAgCaP)
Method: Standard Multivariable Logistic Regression
AUC is 0.73 (0.68-0.79), ROC curve is shown in Figure Fourteen

### Standard Logistic Regression Developed on the CUSP 302 Population Using WFDC2(HE4), PSA, % Free PSA the Abbott Architect Model 44

Model developed to differentiate AgCaP vs NOTAgCaP
Data for model development: all patients (109 AgCaP, 193 NOTAgCaP)
Data for performance report: all patients (109 AgCaP, 193 NOTAgCaP)
Method: Standard Multivariable Logistic Regression
AUC is 0.81 (0.76-0.86), ROC curve is shown in Figure Fifteen

Model using WFDC2(HE4), PSA, % free PSA, PV, Age on the CUSP 302 samples measured on the Abbott platform (Model 46).

### Standard Logistic Regression Developed on the CUSP 302 Population Measured Using the Abbott Platform

Model developed to differentiate AgCaP vs NOTAgCaP
Data for model development: all patients (109 AgCaP, 193 NOTAgCaP)
Data for performance report: all patients (109 AgCaP, 193 NOTAgCaP)
Method: Standard Multivariable Logistic Regression
AUC is 0.86 (0.82-0.9), ROC curve is shown in Figure Sixteen

TABLE 17
Variables, transformations and Log Odds ratio for Model 46

| Variable | Transformation | Log Odd ratio |
|---|---|---|
| (Intercept) | | 3.09086918052 |
| Total.PSA | Log | 1.52445761132 |
| % Free.PSA | Log | −1.07708905573 |
| HE4 (pmol/L) | Log | −0.35058317998 |
| Age | | 0.08638096121 |
| PV | Log | −1.96612204558 |

TABLE 18
Sensitivity, Specificity and Accuracy
for Model 46 at different Thresholds

| | Sensitivity | Specificity | Accuracy |
|---|---|---|---|
| Threshold | (%) | (%) | (%) |
| 0.20236979117 | 90 | 62 | 72.2 |
| 0.19091248858 | 91 | 60 | 71.2 |
| 0.18579653642 | 92 | 60 | 71.2 |
| 0.16724140717 | 93 | 57 | 69.9 |
| 0.16703767216 | 94 | 57 | 70.2 |
| 0.15306188312 | 95 | 53 | 68.2 |

Model using PSA on the CUSP 300 samples measured on the Roche platform (Model 43).

### Standard Logistic Regression Developed on the CUSP 300 Population Measured Using the Roche

Model developed to differentiate AgCaP vs NOTAgCaP
Data for model development: all patients (109 AgCaP, 191 NOTAgCaP)
Data for performance report: all patients (109 AgCaP, 191 NOTAgCaP)
Method: Standard Multivariable Logistic Regression
AUC is 0.72 (0.66-0.78), ROC curve is shown in Figure Seventeen

Model using WFDC2(HE4), PSA, % free PSA on the CUSP 300 samples measured on the Roche platform (Model 48).

### Standard Logistic Regression Developed on the CUSP 300 Population Measured Using the Roche

Model developed to differentiate AgCaP vs NOTAgCaP
Data for model development: all patients (109 AgCaP, 191 NOTAgCaP)
Data for performance report: all patients (109 AgCaP, 191 NOTAgCaP)
Method: Standard Multivariable Logistic Regression
AUC is 0.81 (0.75-0.86), ROC curve is shown in Figure Eighteen

Model using WFDC2(HE4), PSA, % free PSA, PV, Age on the CUSP 300 samples measured on the Roche platform (Model 50).

### Standard Logistic Regression Developed on the CUSP 300 Population Measured Using the Roche

Model developed to differentiate AgCaP vs NOTAgCaP
Data for model development: all patients (109 AgCaP, 191 NOTAgCaP)
Data for performance report: all patients (109 AgCaP, 191 NOTAgCaP)
Method: Standard Multivariable Logistic Regression
AUC is 0.85 (0.81-0.9), ROC curve is shown in Figure Nineteen

TABLE 19
Variables, transformations and Log Odds ratio for Model 50

| Variable | Transformation | Log Odd ratio |
|---|---|---|
| (Intercept) | | 2.47538423691 |
| Total.PSA | Log | 1.55314005574 |
| % Free.PSA | Log | −1.25561079578 |
| HE4 (pmol/L) | Log | −0.05605560121 |
| Age | | 0.07741794309 |
| PV | Log | −1.96574559332 |

TABLE 20
Sensitivity, Specificity and Accuracy
for Model 50 at different Thresholds

| | Sensitivity | Specificity | Accuracy |
|---|---|---|---|
| threshold | (%) | (%) | (%) |
| 0.21815701620 | 90 | 63 | 73.0 |
| 0.20612942366 | 91 | 61 | 71.7 |
| 0.19381710754 | 92 | 58 | 70.3 |
| 0.15700689698 | 93 | 49 | 65.0 |
| 0.15327977054 | 94 | 48 | 64.7 |
| 0.12575556875 | 95 | 40 | 60.3 |

### Development of Cross-Validated Models

The preferred variable combinations of WFDC2(HE4), PSA, % free PSA and Age (pre-MRI) and WFDC2(HE4), PSA, % free PSA, PV and Age (post-MRI) were subjected to Monte Carlo cross-validation for the model development to avoid overfitting. The data was randomly split into two thirds for training data and one third for test data, and the split was repeated for 2000 times. The ratio of different disease groups (AgCaP vs NOT-AgCaP) was maintained the same in the training and test data set. For each split, a multivariable logistic regression model consisting of either 4 variables (i.e. WFDC2(HE4), PSA, % free PSA and Age) or 5 variables (WFDC2(HE4), PSA, % free PSA, PV and Age) were developed using the training data set. The model was then compared in the complementary test data set to get the performance (such as AUC, sensitivity and specificity). The optimal model was selected if its performance was closest to the averaged performance of the 2000 models in the training set and also similar to the average performance in the test dataset. In addition, the model was limited with no more than 6% missed AgCaP with GS≥3+4, and 0% missed GS≥8 the whole population. Amongst models meeting the above criteria, the final best model chosen based on highest AUC and highest specificity at 94% sensitivity.

TABLE 21
Performance of cross-validated models developed on either the MQ 192
sample set, the CUSP sample set, or the combined CUSP + MQ sample sets

| | | | | | | | Specificity | Specificity | Specificity |
|---|---|---|---|---|---|---|---|---|---|
| | | Population | # | | | | at 94% | at 92% | at 90% |
| Model | Components | Developed | Patients | Platform | Model Type | AUC | Sensitivity | Sensitivity | Sensitivity |
| 71 | WFDC2 (HE4), PSA, % free | MQ192 | 192 | Abbott | Cross validated | 0.79 (0.73-0.86) | 39 | 42 | 50 |
| | PSA + PV + Age | | | | | | | | |
| 72 | WFDC2 (HE4), PSA, % free | MQ192 | 302 | Abbott | Cross validated | 0.83 (0.78-0.88) | 47 | 50 | 51 |
| | PSA + PV + Age | | | | | | | | |
| 73 | WFDC2 (HE4), PSA, % free | MQ192 | 49 | Abbott | Cross validated | 0.8 (0.65-0.95) | 92/68 | 83/68 | 75/78 |
| | PSA + PV + Age | | | | | | | | |
| 74 | WFDC2 (HE4), PSA, % free | MICheck 302 | 302 | Abbott | Cross validated | 0.86 (0.82-0.9) | 57 | 60 | 63 |
| | PSA + PV + Age | | | | | | | | |
| 75 | WFDC2 (HE4), PSA, % free | MQ192 | 192 | Abbott | Cross validated | 0.78 (0.72-0.85) | 38 | 39 | 41 |
| | PSA + PV + PIRADS + Age | | | | | | | | |
| 76 | WFDC2 (HE4), PSA, % free | MQ192 | 49 | Abbott | Cross validated | 0.78 (0.62-0.94) | 92/49 | 83/78 | 75/78 |
| | PSA + PV + PIRADS + Age | | | | | | | | |
| 77 | WFDC2 (HE4), PSA, % free | MQ192 + | 506 | Abbott | Cross validated | 0.78 (0.74-0.82) | 40 | 43 | 45 |
| | PSA + Age | MICheck 314 | | | | | | | |
| 78 | WFDC2 (HE4), PSA, % free | MQ192 + | 192 | Abbott | Cross validated | 0.71 (0.63-0.79) | 29 | 30 | 31 |
| | PSA + Age | MICheck 314 | | | | | | | |
| 79 | WFDC2 (HE4), PSA, % free | MQ192 + | 314 | Abbott | Cross validated | 0.82 (0.77-0.87) | 49 | 54 | 57 |
| | PSA + Age | MICheck 314 | | | | | | | |
| 80 | WFDC2 (HE4), PSA, % free | MICheck 302 | 302 | Abbott | Cross validated | 0.82 (0.78-0.87) | 55 | 55 | 56 |
| | PSA + Age | | | | | | | | |

### (a) Post-MRI Cross Validated Model Performance Cross-Validated Model Developed on MQ192 Population (Model 71)

Model developed to differentiate AgCaP vs NOTAgCaP
Data for model development: 52 AgCaP, 77 NOTAgCaP
Data for model performance: 77 AgCaP, 115 NOTAgCaP
Method: cross-validated standard Multivariable Logistic Regression
AUC is 0.79 (0.73-0.86), ROC Curve is shown in Figure Twenty

TABLE 22
Variables, transformations and Log Odds ratio for Model 71

| Variable | Transformation | Log Odd ratio |
|---|---|---|
| (Intercept) | | 5.33118408829 |
| Total.PSA | Log | 0.35665793560 |
| % Free.PSA | Log | −0.60600842967 |
| HE4 (pmol/L) | Log | −0.84912964671 |
| Age | | 0.09225312566 |
| PV | Log | −1.92384410063 |

TABLE 23
Sensitivity, Specificity and Accuracy
for Model 71 at different Thresholds

| | Sensitivity | Specificity | Accuracy |
|---|---|---|---|
| Threshold | (%) | (%) | (%) |
| 0.24568152297 | 90 | 50 | 65.6 |
| 0.20753435499 | 91 | 43 | 62.0 |
| 0.19645681951 | 92 | 42 | 62.0 |
| 0.18867425825 | 94 | 39 | 60.9 |
| 0.17804581054 | 95 | 37 | 59.9 |

A cross-validated model (Model 71) using the preferred post-MRI combination of WFDC2(HE4), PSA, % free PSA, PV and Age was developed using the MQ192 population then applied to the CUSP 302 and PIRADs 1-3 MQ populations. Performance of the cross-validated algorithm was superior compared to the standard algorithm (Model 11) when applied to the MQ192 sample set: Sensitivity 94%, specificity 39% vs. 37%, Sensitivity 92%, 42% specificity vs 39%, Sensitivity 90%, 50% specificity vs 44%).

When applied to the CUSP sample set with available PV data measured on the Abbott platform (Model 72), the cross-validated algorithm developed on the MQ192 sample set showed good performance (AUC 0.83 (0.78-0.88), Sensitivity/specificity of 94%/47%, 92%/50% and 90%/51%). The resulting ROC curve is shown in Figure Twenty One.

TABLE 24
Sensitivity, Specificity and Accuracy
for Model 72 at different Thresholds

| | Sensitivity | Specificity | Accuracy |
|---|---|---|---|
| Threshold | (%) | (%) | (%) |
| 0.25207982309 | 90 | 51 | 65.2 |
| 0.24930519024 | 91 | 51 | 65.6 |
| 0.24023069936 | 92 | 50 | 64.9 |
| 0.23135363503 | 93 | 48 | 64.2 |
| 0.22702266043 | 94 | 47 | 63.9 |
| 0.16943954107 | 95 | 30 | 53.6 |

The cross-validated model developed using the MQ192 sample set was applied to the 41 PIRADs 1-3 patient and 8 patient samples with PV available (Model 73). The performance of the cross-validated model was superior to the standard linear regression mode developed on the 49 patients 1 (Model 37), AUC 0.8 (0.65-0.95) vs 0.77 (0.62-0.92), Sensitivity/Specificity 92%/68% vs 92%/51%, 83%/68% vs 83%/57%, 75%/78% vs 75%/65%. The ROC curve is shown in Figure Twenty Two.

The cross-validated model developed using the MQ192 sample set was applied to the 23 PIRADs 3 samples. It showed AUC 0.72 (0.49-0.96) and sensitivity of 89% (8 of 9 aggressive cancers) and specificity of 64% (9 of 14 true negative patients).

### (b) Pre-MRI Cross Validated Model Performance Cross-Validated Model Developed on MQ192+CUSP 314 Population

A cross-validated model using the preferred pre-MRI combination of WFDC2(HE4), PSA, % free PSA and Age was developed using the combined MQ 192 and CUSP 314 data sets (Model 77).
Model developed to differentiate AgCaP vs NOTAgCaP
Data for model development: 127 AgCaP, 211 NOTAgCaP
Data for model performance: 190 AgCaP, 316 NOTAgCaP
Method: cross-validated standard Multivariable Logistic Regression
AUC is 0.78 (0.74-0.82), ROC Curve is shown in Figure Twenty Three

TABLE 25
Variables, transformations and Log Odds ratio for Model 77

| Variable | Transformation | Log Odd ratio |
|---|---|---|
| (Intercept) | | −0.18674647849 |
| Age | | 0.07529468981 |
| Total.PSA | Log | 0.70319237028 |
| % Free.PSA | Log | −1.61358883504 |
| HE4 (pmol/L) | Log | −0.46278747353 |

TABLE 26
Sensitivity, Specificity and Accuracy
for Model 77 at different Thresholds

| | Sensitivity | Specificity | Accuracy |
|---|---|---|---|
| Cutpoint | (%) | (%) | (%) |
| 0.2199096099 | 90 | 45 | 61.9 |
| 0.2160816033 | 91 | 45 | 61.9 |
| 0.2096301552 | 92 | 43 | 61.3 |
| 0.2057715171 | 93 | 42 | 60.9 |
| 0.1982147939 | 94 | 40 | 59.9 |
| 0.1940203479 | 95 | 39 | 59.7 |

When applied to the MQ192 sample set (model 78), this cross-validated model had lower AUC 0.71 (0.63-0.79) vs 0.73 (0.66-0.81) compared to the standard model developed on the MQ192 sample set. However, the cross-validated model had superior sensitivity/specificity in the MQ 192 sample set to the standard logistic regression Model 16 (94% sensitivity, 29% vs 16% specificity, 92% sensitivity, 30% vs 28% specificity, 90% sensitivity 31% vs 30% specificity). The ROC curve is shown in Figure Twenty Four.

TABLE 27
Sensitivity, Specificity and Accuracy
for Model 78 at different Thresholds

| | Sensitivity | Specificity | Accuracy |
|---|---|---|---|
| cutpoint | (%) | (%) | (%) |
| 0.20626589070 | 90 | 31 | 54.7 |
| 0.20577151707 | 91 | 31 | 55.2 |
| 0.19402034792 | 92 | 30 | 55.2 |
| 0.18376046303 | 94 | 29 | 54.7 |
| 0.17645965505 | 95 | 26 | 53.6 |

When the 506 cross-validated model was applied to the CUSP 314 population (Model 79) it showed high performance: AUC 0.82 (0.77-0.87), Sensitivity/Specificity of 94%/49%, 92%/54% and 90%/57%). The ROC curve is shown in Figure Twenty Five.

TABLE 28
Sensitivity, Specificity and Accuracy
for Model 79 at different Thresholds

| | Sensitivity | Specificity | Accuracy |
|---|---|---|---|
| Cutpoint | (%) | (%) | (%) |
| 0.24986146707 | 90 | 57 | 69.1 |
| 0.24895336166 | 91 | 57 | 69.4 |
| 0.22639105703 | 92 | 54 | 67.5 |
| 0.22630555479 | 93 | 54 | 67.8 |
| 0.20963015521 | 94 | 49 | 65.3 |
| 0.19981019180 | 95 | 45 | 63.1 |
| 0.19821479394 | 96 | 44 | 62.7 |
| 0.18236498798 | 97 | 40 | 60.8 |

### Clinical Utility of Post-MRI MiCheck® Prostate

Table 29 shows the clinical performance and the biopsy outcomes of MiCheck® Prostate post-MRI algorithm applied to the MQ192 population (Model 71) using a 94% sensitivity cutpoint. The percentage biopsies saved are shown in Figure Twenty Six.

TABLE 29
Algorithm classifications for MQ192 using the best post-MRI model
Algorithm Outcomes

| | Of 142 positive | Of 50 negative |
|---|---|---|
| Diagnosis | MiCheck tests | MiCheck tests |
| No Cancer | 49 | 29 |
| Non Aggressive CaP | 21 | 16 |
| Aggressive CaP | 72 | 5 (5 GS 3 + 4, |
| | | 0 GS ≥ 3 + 4) |

39% of unnecessary biopsies saved
Negative Predictive Value (GS≥3+4)=90.0%
Negative Predictive Value (GS≥4+3)=100.0%
6.49% GS≥3+4 cancers delayed diagnosis
0% GS≥4+3 cancers delayed diagnosis
0% GS≥8 cancers delayed diagnosis

### Clinical Utility of MiCheck® Prostate Prior to MRI

Table 30 shows the clinical performance and the biopsy outcomes of MiCheck® Prostate post-MRI algorithm applied to the MQ192 population (Model 78) using a 92% sensitivity cutpoint. The percentage biopsies saved are shown in Figure Twenty Seven

TABLE 30
Algorithm classifications for MQ192 using the best pre-MRI model.
Algorithm Outcomes

| | Of 151 positive | Of 41 negative |
|---|---|---|
| Diagnosis | MiCheck tests | MiCheck tests |
| No Cancer | 54 | 24 |
| Non Aggressive CaP | 26 | 11 |
| Aggressive CaP | 71 | 6 (4 GS 3 + 4, |
| | | 2 GS 4 + 3) |

30% of unnecessary biopsies saved
Negative Predictive Value (GS≥3+4)=85.4%
Negative Predictive Value (GS≥4+3)=95.1%
7.79% GS≥3+4 cancers delayed diagnosis
5.88% GS≥4+3 cancers delayed diagnosis
0% GS≥8 cancers delayed diagnosis

### Superiority of MiCheck® Prostate MRI (Model 73) and MiCheck® Prostate Non-MRI (Model 79) to PSA

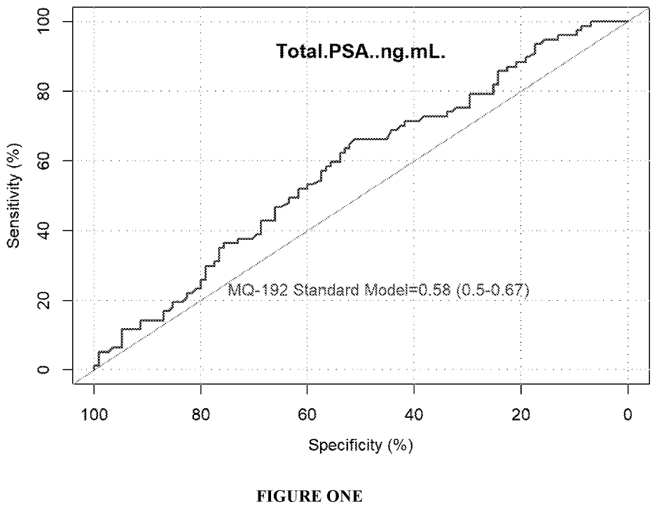

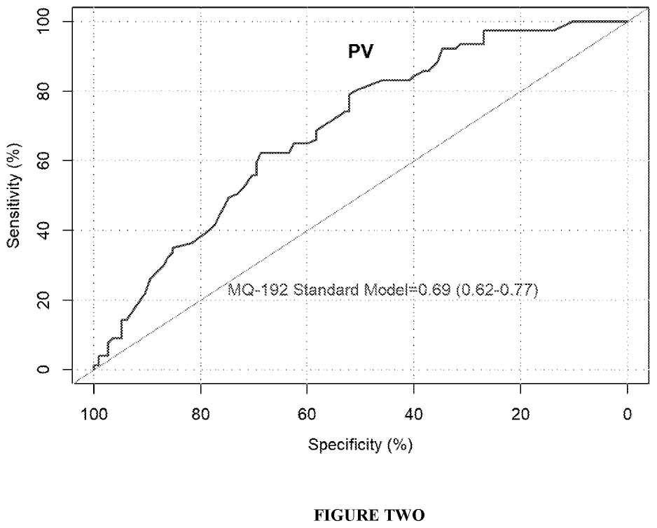

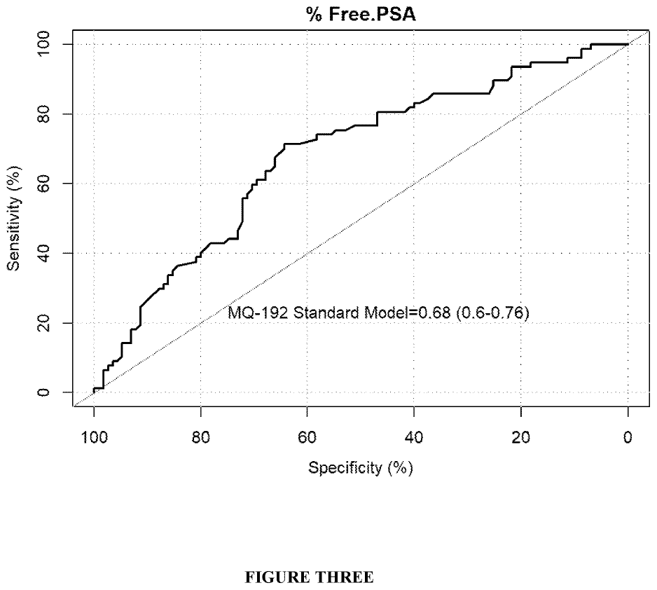

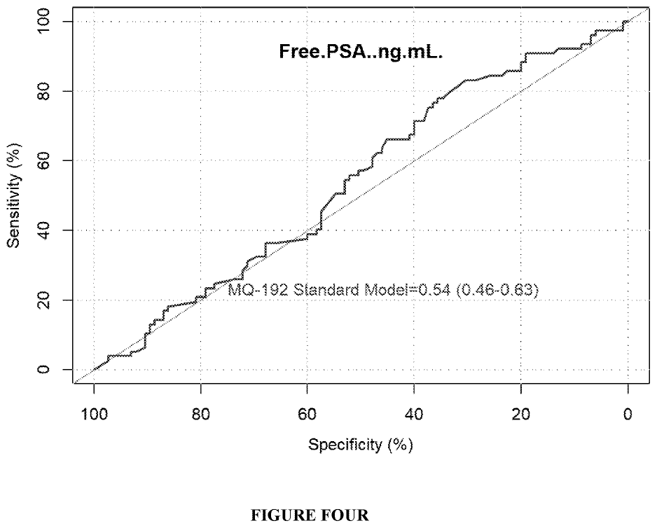

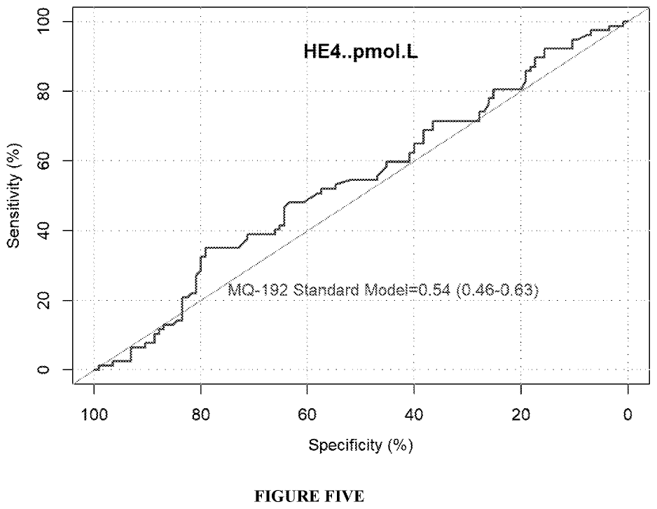

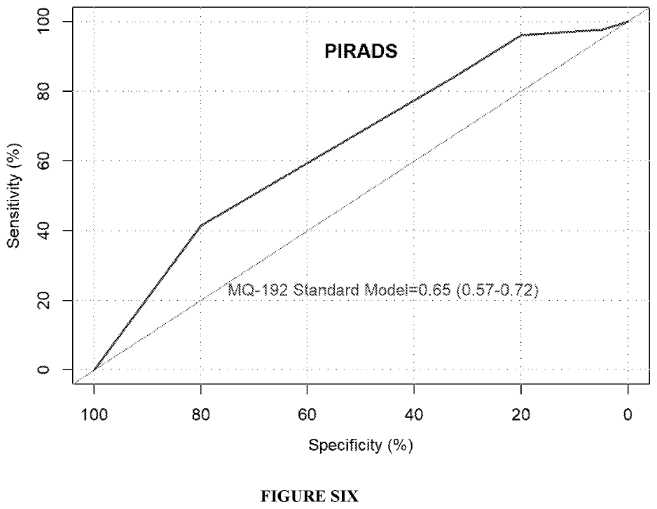

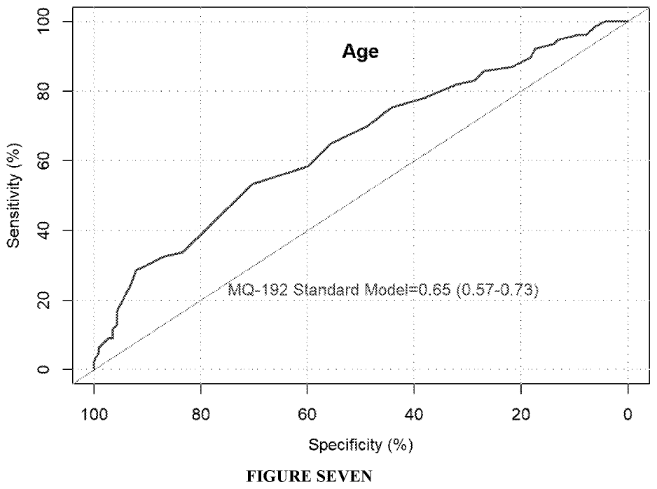

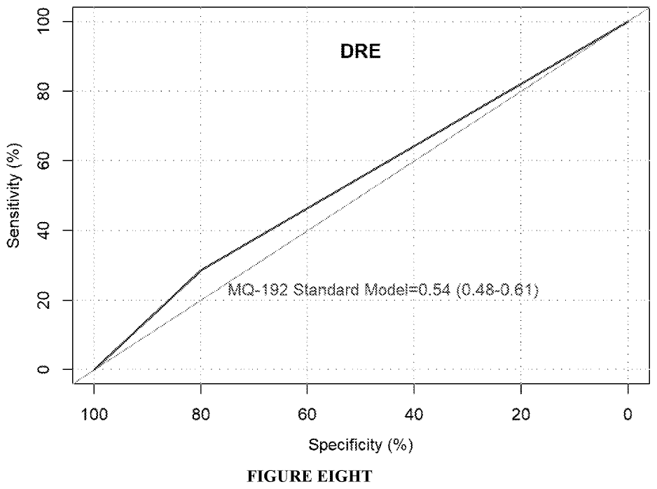

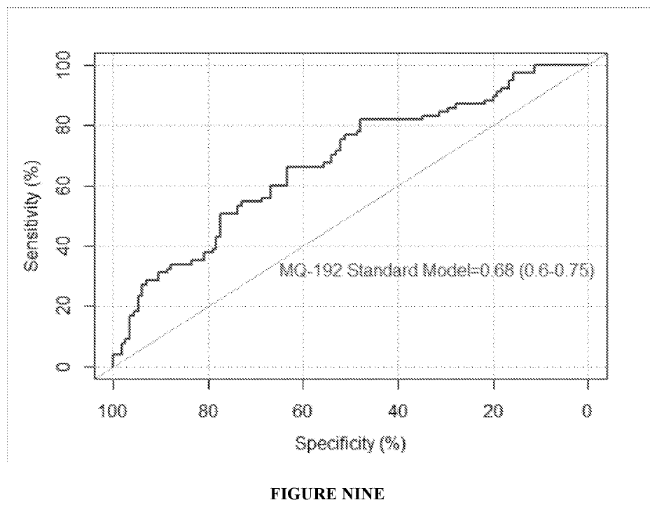

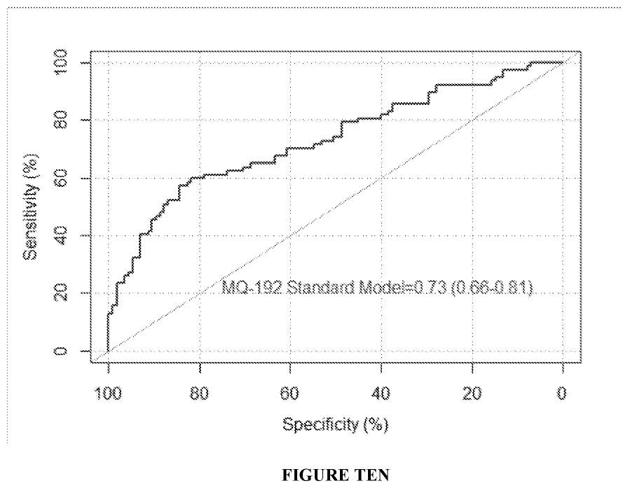

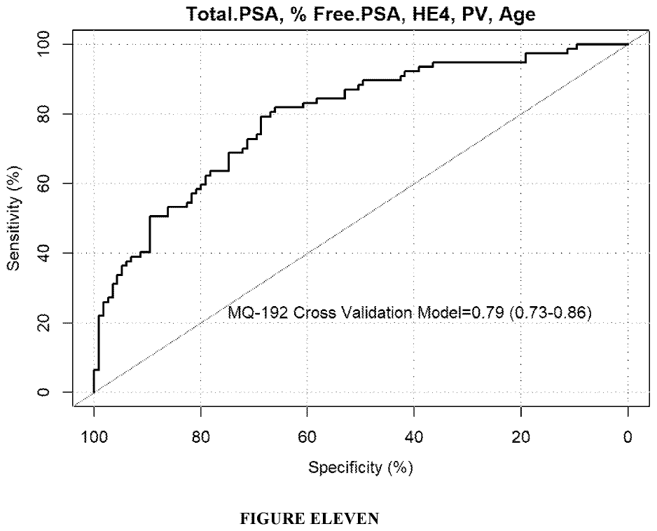

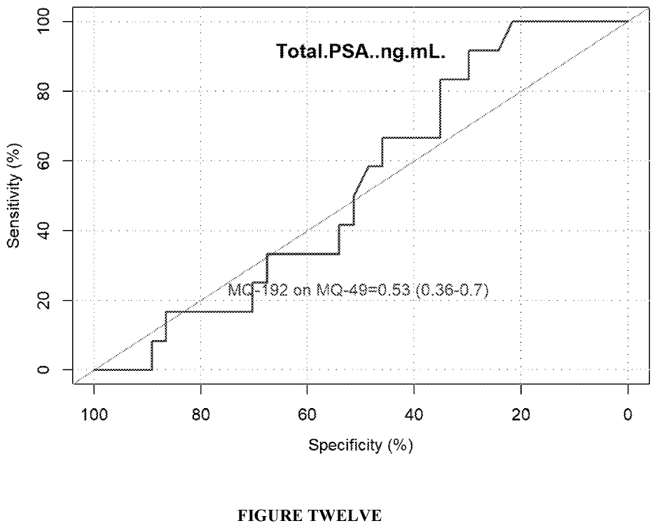

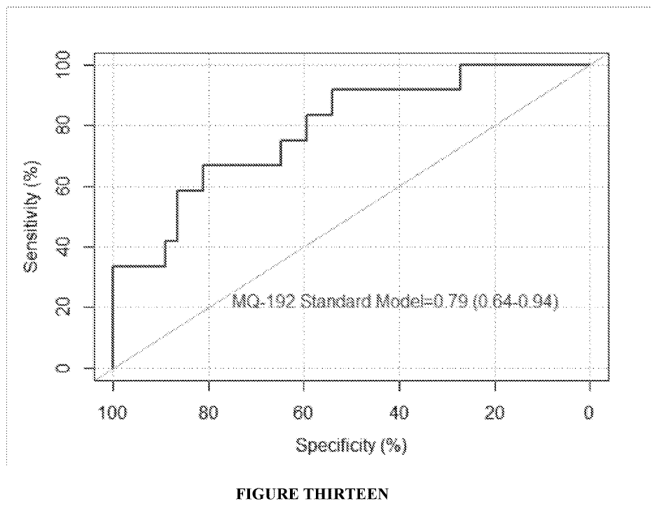

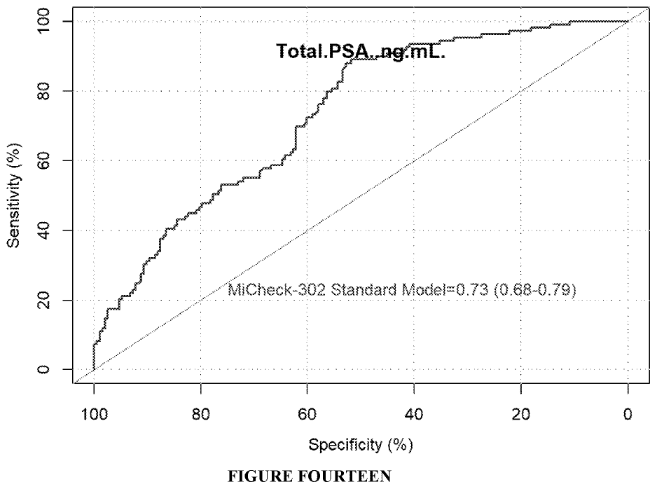

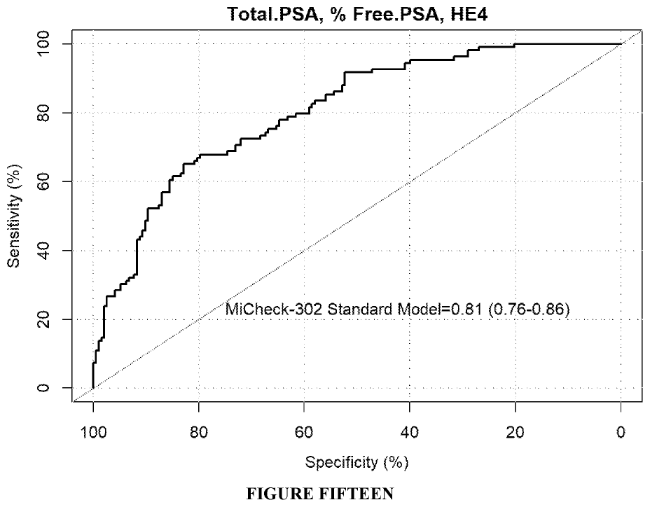

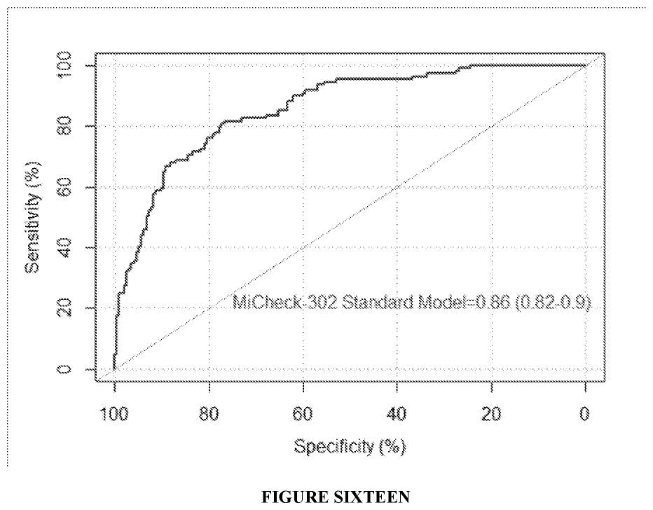

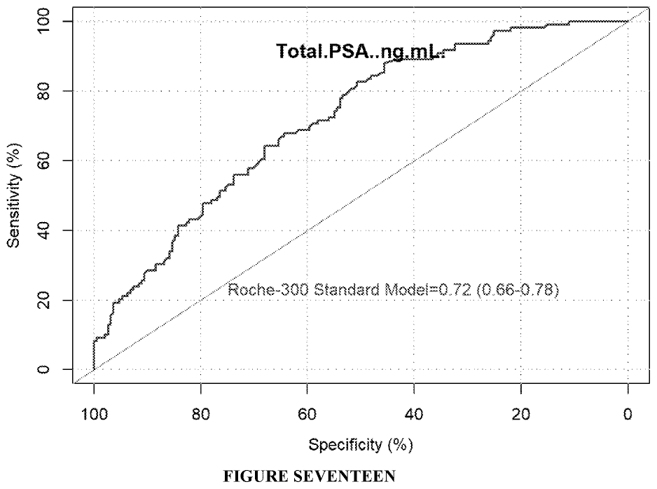

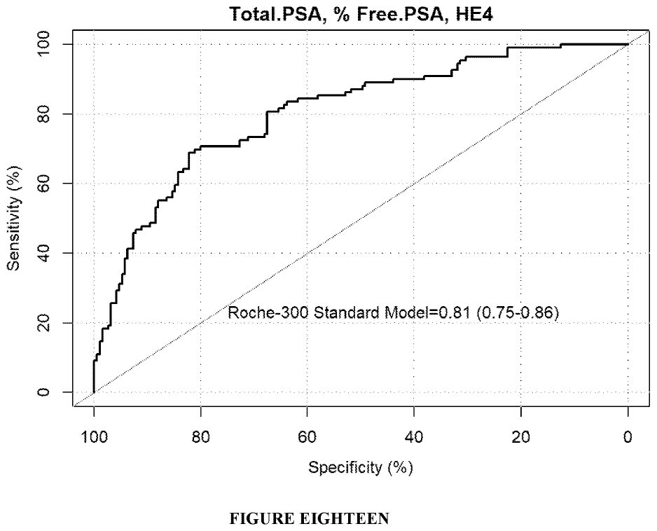

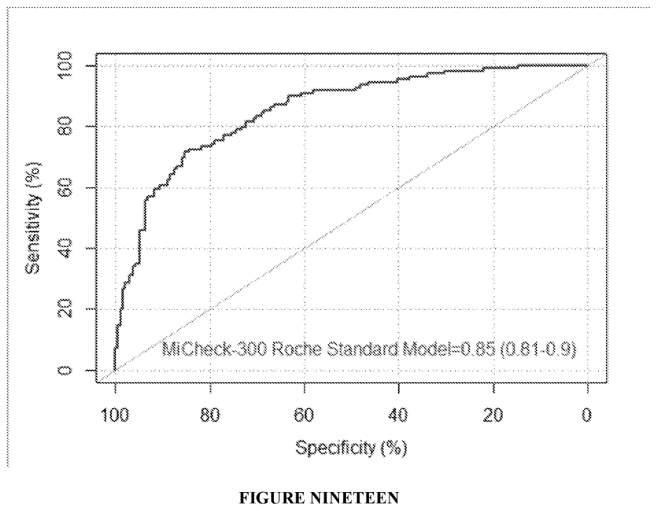

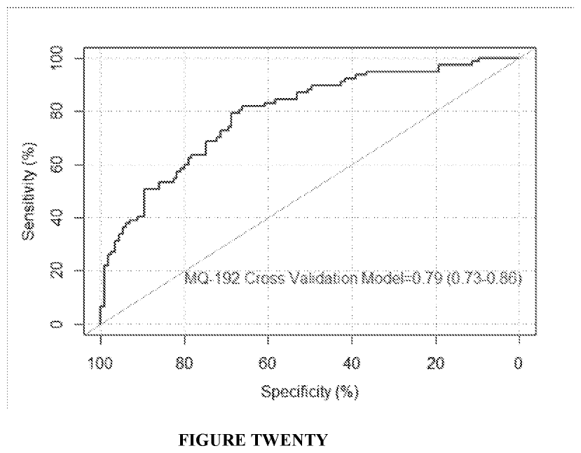

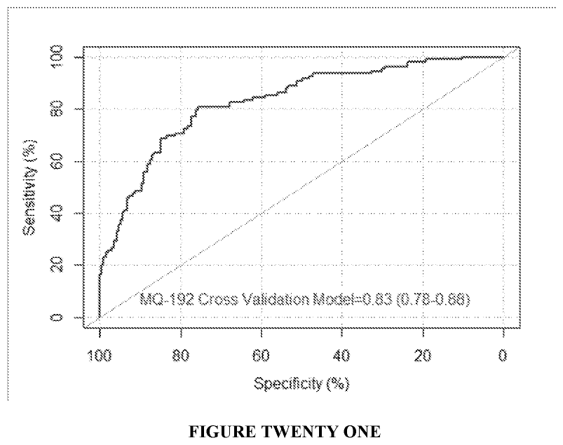

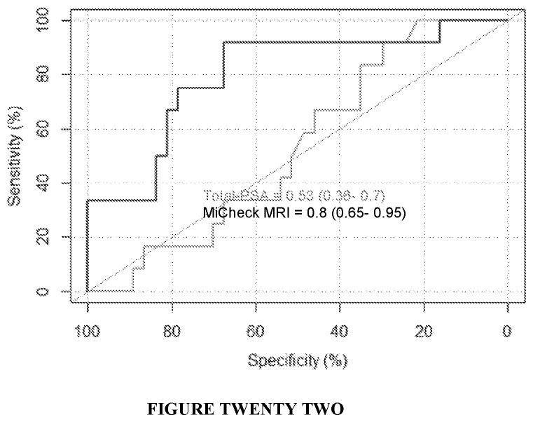

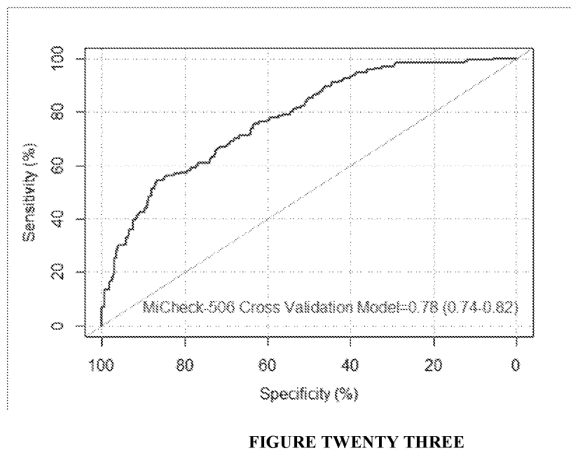

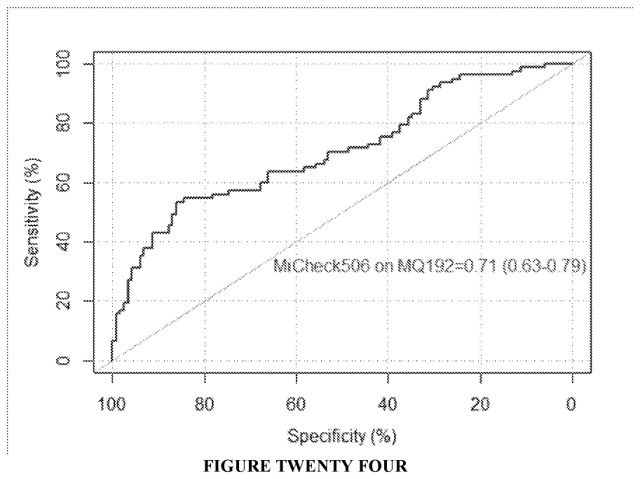

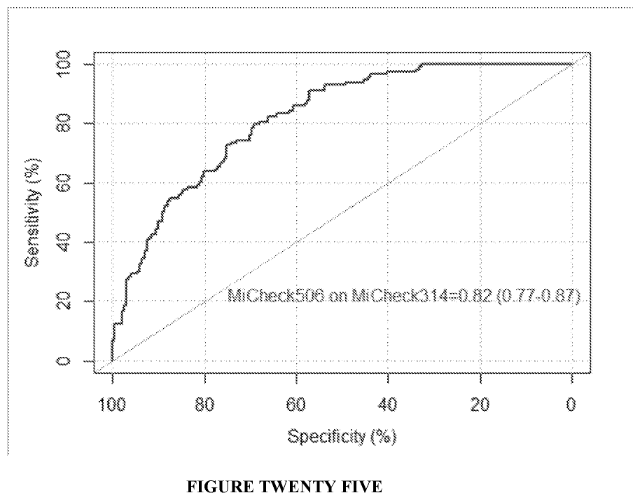

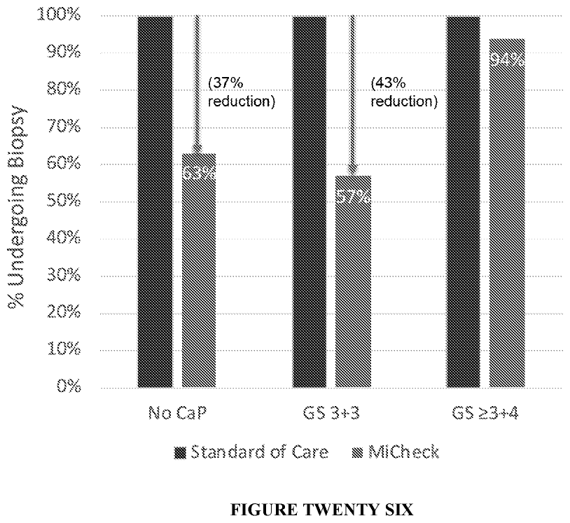

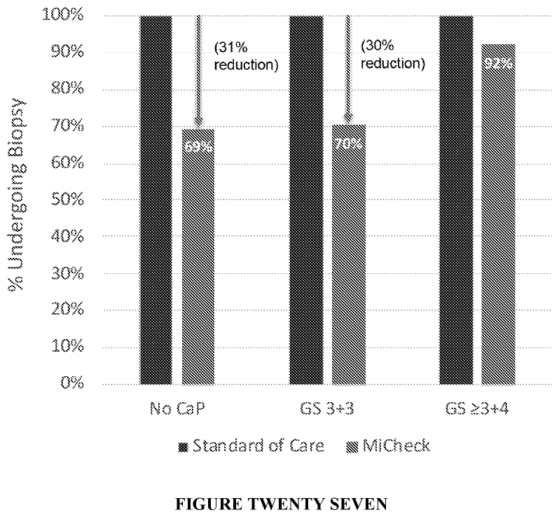

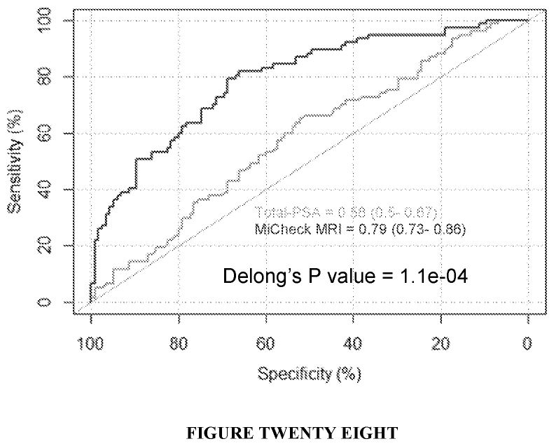

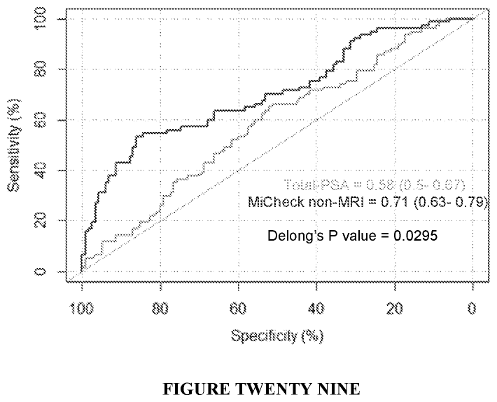

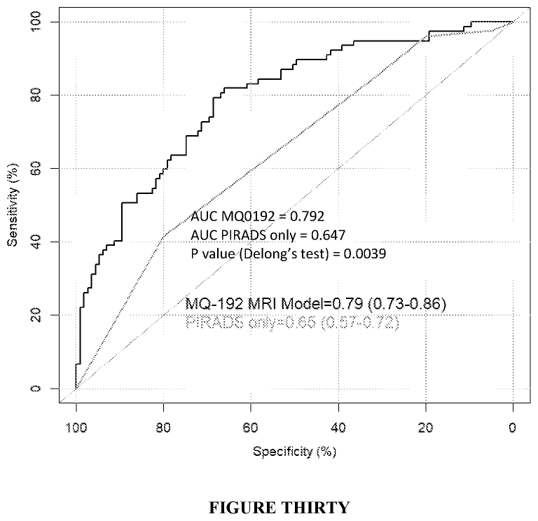

ROC curve comparisons were performed comparing the performance of PSA with MiCheck® Prostate MRI (FIG. 28) and MiCheck® Prostate non-MRI (FIG. 29) models. Both models were statistically significantly superior to PSA (P=1.1e-04 and p=0.0295 respectively).

### Superiority of MiCheck® Prostate MRI (Model 73) to PIRADs (Model 6)

Under current clinical workflows (Section 1.1) a urologist will make biopsy decision on the basis of PIRADs scores. If an MRI has been performed, MRI derived PV data will be available, hence MiCheck® Prostate MRI can be performed prior to making a biopsy decision. The performance of MiCheck® Prostate MRI for the detection of clinically significant cancer was compared the performance of MRI alone.

A ROC curve comparison was performed comparing the performance of MiCheck® Prostate MRI (model 73) and PIRADs (model 6). MiCheck® Prostate MRI was statistically significantly better than PIRADS (p=0.0039).

### MiCheck® Prostate MRI Test Performance in Low PIRADs Patients

Patients who present with MRI PIRADs scores of 4 or 5 will typically proceed to prostate biopsy. Patients with PIRADs scores of 1 or 2 will often not proceed to biopsy, despite up to 18% of these patients having clinically significant prostate cancer (Doan et al, Identifying prostate cancer in men with non-suspicious multi-parametric magnetic resonance imaging of the prostate. ANZ J Surg 2021; 91:578-83. https://doi.org/10.111 1ANS.16583). Patients with PIRADs scores of 3 represent a particularly challenging subgroup as clinically significant cancer rates may be as low as 12% (Eklund et al MRI-Targeted or Standard Biopsy in Prostate Cancer Screening. New England Journal of Medicine 2021; 385:908-20. https://doi.org/10.1056/NEJMOA2100852/SUPPL FILE/NEJMOA2100852 DATA-SHARING.PDF, Mazzone et al Positive Predictive Value of Prostate Imaging Reporting and Data System Version 2 for the Detection of Clinically Significant Prostate Cancer: A Systematic Review and Meta-analysis. Eur. Urol. Oncol 2021; 4:697-713. https://doi.org/10.1016/J.EUO.2020.12.004).

The performance of MiCheck® Prostate (using cutpoints for 94% or 90% sensitivity in the whole MQ population) was compared in the whole MQ 192 population, and in the PIRADs 1-3 and PIRADs 4 and 5 populations (Table 31). Of particular note, performance of 92% sensitivity and 65% specificity was observed in the PIRADS 1-3 population representative of the significant clinical unmet need (Table 31, B).

TABLE 31
Comparison of MiCheck ® Prostate MRI model 73 in the
whole MQ192 population and in PIRADs 1-3 and PIRADS 4, 5
subgroups of the MQ-192 population.
Test performance was assessed at 94% sensitivity (A-C) or 90%
sensitivity (D-F) for either the whole MQ 192 population
(A, D), the PIRADs 1-3 population (B, E) or the PIRADs
4 and 5 population (C, D).
At 94% sensitivity threshold 0.18867425825

| | B. PIRADS 1-3 | C. PIRADS 4-5 |
|---|---|---|
| A. MQ-192 | (n = 49) | (n = 143) |

| TP | FP | TP | FP | TP | FP |
|---|---|---|---|---|---|
| 72 | 70 | 11 | 13 | 61 | 57 |
| 5 | 45 | 1 | 24 | 4 | 21 |
| FN | TN | FN | TN | FN | TN |
| Sensitivity | 94% | Sensitivity | 92% | Sensitivity | 94% |
| Specificity | 39% | Specificity | 65% | Specificity | 27% |
| NPV | 90% | NPV | 96% | NPV | 84% |
| PPV | 51% | PPV | 46% | PPV | 52% |

At 90% sensitivity threshold 0.24568152297

| | E. PIRADS 1-3 | F. PIRADS 4-5 |
|---|---|---|
| D. MQ-192 | (n = 49) | (n = 143) |

| TP | FP | TP | FP | TP | FP |
|---|---|---|---|---|---|
| 69 | 58 | 9 | 10 | 60 | 48 |
| 8 | 57 | 3 | 27 | 5 | 30 |
| FN | TN | FN | TN | FN | TN |
| Sensitivity | 90% | Sensitivity | 75% | Sensitivity | 92% |
| Specificity | 50% | Specificity | 73% | Specificity | 38% |
| NPV | 88% | NPV | 90% | NPV | 86% |
| PPV | 54% | PPV | 47% | PPV | 56% |

TP = True Positive, FP = False Positive, FN = False Negative, TN = True Negative, NPV = Negative Predictive Value, PPV = Positive Predictive Value.

A further breakdown into different PIRADs groups was conducted (Table 32). MiCheck® Prostate MRI had 100% sensitivity and 65% specificity for PIRADs 1 and 2 patients (who would not normally proceed to biopsy, Table 32A). For PIRADs 3 patients, test performance was 89% sensitivity and 64% specificity, with only 1 false negative test result (this patient was a low grade Gleason 3+4 cancer). This suggests that in PIRADs 3 patients, a positive MiCheck® Prostate MRI test result could assist in identifying those patients who do require a prostate biopsy.

For PIRADs 4 and 5 patients, test sensitivity was high (94%, Table 32C and D), with two false negative test results per group (all of which were GS 3+4 cancers). In the PIRADs 4 group (the largest individual PIRADs group) the negative predictive value of the test was 87%, with only 2 false negatives (both Gleason 3+4, Table 32C). This suggests that the MiCheck® Prostate MRI test could assist in identifying those patients who may not require a prostate biopsy, or whose biopsy could be delayed.

TABLE 32
Comparison of MiCheck ® Prostate MRI model 73 in the PIRADs 1-2,
PIRADs 3, PIRADS 4 and PIRADs 5 subgroups of the MQ-192 population.
At 94% sensitivity threshold 0.18867425825

| A. PIRADS 1-2 (n = 26) | | B. PIRADS 3 (n = 23) | | C. PIRADS 4 (n = 88) | | D. PIRADS 5 (n = 55) | |
|---|---|---|---|---|---|---|---|
| TP | FP | TP | FP | TP | FP | TP | FP |
| 3 | 8 | 8 | 5 | 31 | 42 | 30 | 15 |
| 0 | 15 | 1 | 9 | 2 | 13 | 2 | 8 |
| FN | TN | FN | TN | FN | TN | FN | TN |
| Sensitivity | 100% | Sensitivity | 89% | Sensitivity | 94% | Sensitivity | 94% |
| Specificity | 65% | Specificity | 64% | Specificity | 24% | Specificity | 35% |
| NPV | 100% | NPV | 90% | NPV | 87% | NPV | 80% |
| PPV | 27% | PPV | 62% | PPV | 42% | PPV | 67% |

At 90% sensitivity threshold 0.24568152297

| E. PIRADS 1-2 (n = 26) | | F. PIRADS 3 (n = 23) | | G. PIRADS 4 (n = 88) | | H. PIRADS 5 (n = 55) | |
|---|---|---|---|---|---|---|---|
| TP | FP | TP | FP | TP | FP | TP | FP |
| 2 | 5 | 7 | 5 | 30 | 34 | 30 | 14 |
| 1 | 18 | 2 | 9 | 3 | 21 | 2 | 9 |
| FN | TN | FN | TN | FN | TN | FN | TN |
| Sensitivity | 67% | Sensitivity | 78% | Sensitivity | 91% | Sensitivity | 94% |
| Specificity | 78% | Specificity | 64% | Specificity | 38% | Specificity | 39% |
| NPV | 95% | NPV | 82% | NPV | 88% | NPV | 82% |
| PPV | 29% | PPV | 58% | PPV | 47% | PPV | 68% |

Test performance was assessed at 94% sensitivity (A-D) or 90% sensitivity (E-H) for either the PIRADS 1-2 population (A, E), the PIRADs 3 population (B, F), the PIRADs 4 (C, G) or the PIRADs 5 population (D, G). TP = True Positive, FP = False Positive, FN = False Negative, TN = True Negative, NPV = Negative Predictive Value, PPV = Positive Predictive Value.

### REFERENCES

1. SEER Cancer Statistics Factsheets: Prostate Cancer. National Cancer Institute. Bethesda, MD, http://seer.cancer.gov/statfacts/html/prost.html
2. Parekh et al. Eur. Urol. 2015 September; 68(3):464-70
3. Carter H B, Albertsen P C, Barry M J et al: Early detection of prostate cancer: AUA Guideline. J Urol. 2013; 190: 419, 2018 update: https://www.auanet.org/guidelines-and-quality/luidelines/prostate-cancer-early-detection-guideline
4. Fleshner & Carlsson, Nature Reviews Urology, volume 15, pages 532-534, 2018
5. Kasivisvanathan V, Rannikko A S, Borghi M, Panebianco V, Mynderse L A, Vaarala M H, Briganti A, Budaus L, Hellawell G, Hindley R G, Roobol M J, Eggener S, Ghei M, Villers A, Bladou F, Villeirs G M, Virdi J, Boxler S, Robert G, Singh P B, Venderink W, Hadaschik B A, Ruffion A, Hu J C, Margolis D, Crouzet S, Klotz L, Taneja S S, Pinto P, Gill I, Allen C, Giganti F, Freeman A, Morris S, Punwani S, Williams N R, Brew-Graves C, Deeks J, Takwoingi Y, Emberton M, Moore C M, PRECISION Study Group Collaborators. MRI-Targeted or Standard Biopsy for Prostate-Cancer Diagnosis. N Engl J Med. 2018; 378(19):1767. Epub 2018 Mar. 18
6. Doan, P, Lahoud J, Kim, L, Patel, MI. identifying prostate cancer in men with non-suspicious multi-parametric magnetic resonance imaging of the prostate. Eur. Urol. 2021 August; 80(2):260-261. Doi: 10.1016/j.eururo.2021.01.039. Epub 2021 Feb. 13.
7. “2018 Annual Report on Prostate Diseases”, Harvard Health Publications (Harvard Medical School
8. Tempany C, Carroll P, Leapman M (2022). The role of magnetic resonance imaging in prostate cancer. https://www.uptodate.com/contents/the-role-of-magnetic-resonance-imaging-in-prostate-cancer?search=PIRADS %20scores&source=search_result&selectedTitle=1˜150&usagetype=default &display_rank=1 accessed 30/9/22
9. Hofbauer SL, Maxeiner A, Kittner B, Heckmann R, Reimann M, Wiemer L, Asbach P, Haas M, Penzkofer T, Stephan C, Friedersdorff F, Fuller F, Miller K, Cash H. Validation of Prostate Imaging Reporting and Data System Version 2 for the Detection of Prostate Cancer. J Urol. 2018; 200(4):767. Epub 2018 May 5.
10. Sonn G A, Fan R E, Ghanouni P, Wang N N, Brooks J D, Loening A M, Daniel BL, To'o K J, Thong A E, Leppert J T. Prostate Magnetic Resonance Imaging Interpretation Varies Substantially Across Radiologists. Eur. Urol. Focus. 2019; 5(4):592
11. Kohler and Milstein, (1975) Nature, 256:495-497
12. Coligan et al. section 2.5.1-2.6.7 in Methods In Molecular Biology (Humana Press 1992)
13. Harlow and Lane Antibodies: A Laboratory Manual, page 726 (Cold Spring Harbor Pub. 1988)
14. Cole, et al. 1985, in Monoclonal Antibodies and Cancer Therapy, Alan R. Liss, Inc., pp. 77-96
15. Kozbor et al. 1983, Immunology Today 4:72
16. Harlow and Lane. Using antibodies. A Laboratory Manual. Cold Spring Harbor, New York: Cold Spring Harbor Laboratory Press, 1999
17. Bold and Mahoney, Analytical Biochemistry 257, 185-192, 1997
18. National Comprehensive Cancer Centre Practice Guidelines in Oncology. Prostate Cancer Version 1.2023—Septermber 16, 2022, nccn.org
19. Neal D Shore, Christopher M Pieczonka, R Jonathan Henderson, James L Bailen, Daniel R Saltzstein, Raoul S Concepcion, Jennifer L Beebe-Dimmer, Julie J Ruterbusch, Thao Ho Le, Rachel A Levin, Sandra Wissmueller, Philip Prah, Robert Borotkanics, Thomas A Paivanas, Arletta van Breda, Douglas H Campbell, Bradley J Walsh. A Comparison of Prostate Health Index, Total PSA, % free PSA, and proPSA in a Contemporary US population—The MiCheck-01 Prospective Trial. Urol. Oncol. 2020 Volume 38, Issue 8, August 2020, Pages 683.e1-683.e10
20. R Core Team (2017). R: A language and environment for statistical computing. R Foundation for Statistical Computing, Vienna, Austria. ISBN 3-900051-07-0, URL http://www.R-project.org/
21. Adrian Raftery, Jennifer Hoeting, Chris Volinsky, Ian Painter and Ka Yee Yeung (2018). BMA: Bayesian Model Averaging. R package version 3.18.8. https://CRAN.R-project.org/package=BMA
22. Genuer, R. and Poggi, J. M. and Tuleau-Malot, C. (2010), Variable selection using random forests, Pattern Recognition Letters 31(14), 2225-2236
23. Genuer, R. and Poggi, J. M. and Tuleau-Malot, C. (2015), VSURF: An R Package for Variable Selection Using Random Forests, The R Journal 7(2):19-33
24. Max Kuhn. Contributions from Jed Wing, Steve Weston, Andre Williams, Chris Keefer, Allan Engelhardt, Tony Cooper, Zachary Mayer, Brenton Kenkel, the R Core Team, Michael Benesty, Reynald Lescarbeau, Andrew Ziem, Luca Scrucca, Yuan Tang, Can Candan and Tyler Hunt. (2018). caret: Classification and Regression Training. R package version 6.0-79. https://CRAN.R-project.org/package=caret
25. Xavier Robin, Natacha Turck, Alexandre Hainard, et al. (2011) “pROC: an open-source package for R and S+ to analyze and compare ROC curves”. BMC Bioinformatics, 7, 77. DOI: 10.1186/1471-2105-12-77
26. Sing T, Sander O, Beerenwinkel N and Lengauer T (2005). “ROCR: visualizing classifier performance in R.” _Bioinformatics, *21*(20), pp. 7881. <URL: http://rocr.bioinf.mpi-sb.mpg.de>
27. Doan P, Lahoud J, Kim L, Patel M I. Identifying prostate cancer in men with non-suspicious multi-parametric magnetic resonance imaging of the prostate. ANZ J Surg 2021; 91:578-83. https://doi.org/10.1111/ANS. 16583
28. Eklund M, Jaderling F, Discacciati A, Bergman M, Annerstedt M, Aly M, et al. MRI-Targeted or Standard Biopsy in Prostate Cancer Screening. New England Journal of Medicine 2021; 385:908-20. https://doi.org/10.1056/NEJMOA2100852/SUPPL FILE/NEJMOA2100852 DATA-SHARING.PDF.
29. Mazzone E, Stabile A, Pellegrino F, Basile G, Cignoli D, Cirulli G O, et al. Positive Predictive Value of Prostate Imaging Reporting and Data System Version 2 for the Detection of Clinically Significant Prostate Cancer: A Systematic Review and Meta-analysis. Eur. Urol. Oncol 2021; 4:697-713. https://doi.org/10.1016/J.EUO.2020.12.004.
30. The Immunoassay Handbook. Theory and applications of ligand binding, ELISA and related techniques”, Wild, D. (Ed), 4th edition, 2013, Elsevier.
31. Harlow and Lane. Using antibodies. A Laboratory Manual. Cold Spring Harbor, New York: Cold Spring Harbor Laboratory Press, 1999; Bold and Mahoney, Analytical Biochemistry 257, 185-192, 1997.

1. A method for detecting aggressive prostate cancer (CaP) in a test subject, comprising:
(a) having obtained an analyte level for one or more analytes in the test subject's biological sample, and having obtained a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject is likely to have aggressive CaP by comparison of the subject test score value and the threshold value,
wherein:
the one or more analyte/s comprise or consist of WAP four-disulfide core domain protein 2 (WFDC2 (HE4)), and optionally total prostate surface antigen (PSA),
the one or more clinical variables comprise at least one of: % Free PSA, Free PSA, DRE, Prostate Volume (PV), PIRADs score, Age, Family History (FH) and
the threshold value was determined by:
measuring said one or more analyte/s in a series of biological samples obtained from a population of subjects having aggressive CaP and from a population of control subjects not having aggressive CaP, to thereby obtain an analyte level for each said analyte in each said biological sample of the series;
combining each said analyte level of the series with measurements of said one or more clinical variables obtained from each said subject of the populations, in a manner that allows discrimination between aggressive CaP and an absence of aggressive CaP, to thereby generate the threshold value.

2. The method according to claim 1, wherein the population of control subjects comprises or consists of subjects that do not have prostate cancer.

3. The method according to claim 1, wherein the population of control subjects comprises or consists of subjects that do not have aggressive prostate cancer.

4. The method of claim 1 according to claim 3, wherein the population of control subjects has non-aggressive CaP as defined by a Gleason score of 3+3 or do not have prostate cancer.

5. The method according to any one of claims 1 to 4, wherein the threshold value is determined prior to performing the method.

6. The method according to any one of claims 1 to 5, wherein the one or more clinical variables and the one or more analyte/s comprise or consist of any one of the following:
WFDC2 (HE4), total PSA, % Free PSA and Age
WFDC2 (HE4), total PSA, % Free PSA, PV and Age
WFDC2 (HE4), total PSA, % Free PSA and PIRADs
WFDC2 (HE4), total PSA, % Free PSA, PV and PIRADs
WFDC2 (HE4), total PSA, % Free PSA, PV, PIRADs and Age
WFDC2 (HE4), total PSA, % Free PSA, PV, PIRADs, Age and DRE
WFDC2 (HE4), total PSA, % Free PSA, Age and DRE
WFDC2 (HE4), total PSA, % Free PSA, Age and FH
WFDC2 (HE4), total PSA, % Free PSA, Age, DRE, FH
WFDC2 (HE4), % Free PSA and Age
WFDC2 (HE4), % Free PSA and PIRADs
WFDC2 (HE4), % Free PSA, PV and Age
WFDC2 (HE4), % Free PSA, PV and PIRADs
WFDC2 (HE4), % Free PSA, PV, Age and PIRADs
WFDC2 (HE4), % Free PSA, PV, Age, PIRADS and DRE
WFDC2 (HE4), total PSA, Free PSA
WFDC2 (HE4), total PSA, Free PSA and PV
WFDC2 (HE4), total PSA, Free PSA, PV and Age
WFDC2 (HE4), total PSA, Free PSA, PV and PIRADs
WFDC2 (HE4), total PSA, Free PSA, PV, PIRADs and Age
WFDC2 (HE4), total PSA, Free PSA and Age
WFDC2 (HE4), total PSA, Free PSA and PIRADs

7. The method according to any one of claims 1 to 6, comprising selecting a subset of the combined analyte/s and/or clinical variable measurements to generate the threshold value.

8. The method according to any one of claims 1 to 7, wherein said combining of each said analyte level of the series with said measurements of the one or more clinical variables comprises combining a logistic regression score of the clinical variable measurements and analyte level/s in a manner that maximizes said discrimination, in accordance with the formula:
$$\begin{matrix}Logit ( P ) = Log ( P / 1 - P ) \\ = intercept + {\sum }_{i = 1}^{N} ( {coefficient}_{i} \times transformed ( {variable}_{i} ) \\ P = \frac{exp ( Logit ( P ) )}{1 + exp ( Logit ( P ) )}\end{matrix} \qquad ( i )$$
wherein:
P is probability of that the test subject has aggressive prostate cancer,
the coefficientᵢ is the natural log of the odds ratio of the variable,
the transformed variableᵢ is the natural log of the variableᵢ value; or
$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( ii )$$
wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable,
the variableⱼ is the numerical value of the variableⱼ; or
$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( iii )$$
wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable,
the variableⱼ is the numerical value of the variableⱼ,
variableⱼ can be one or more of DRE value, Age, or PIRADS score,
if variableⱼ is DRE, a DRE value of 1 indicates an abnormal DRE status and a DRE value of 0 indicates a normal DRE status,
if variableⱼ is PIRADs score, the PIRADS score is 1, 2, 3, 4 or 5, and if a PIRADS score is not available, variableⱼ is 0, and
if variableⱼ is Age, the Age is in years.

9. The method according to any one of claims 1 to 8, wherein said applying a suitable algorithm and/or transformation to the combination of the clinical variable measurements and analyte level/s comprises use of an exponential function, a logarithmic function, a power function and/or a root function.

10. The method according to any one of claims 1 to 9, wherein the suitable algorithm and/or transformation applied to the combination of the clinical variable measurements and analyte level/s of the test subject is in accordance with the formula:
$$\begin{matrix}Logit ( P ) = Log ( P / 1 - P ) \\ = intercept + {\sum }_{i = 1}^{N} ( {coefficient}_{i} \times transformed ( {variable}_{i} ) \\ P = \frac{exp ( Logit ( P ) )}{1 + exp ( Logit ( P ) )}\end{matrix} \qquad ( i )$$
wherein:
P is probability of that the test subject has aggressive prostate cancer,
the coefficientᵢ is the natural log of the odds ratio of the variable,
the transformed variableᵢ is the natural log of the variableᵢ value; or
$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( ii )$$
wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable,
the variableⱼ is the numerical value of the variableⱼ; or
$$\begin{matrix}Logit ( P ) = Log ( P / 1 - P ) \\ = intercept + {\sum }_{i = 1}^{N} ( {coefficient}_{i} \times transformed ( {variable}_{i} ) + \\ {\sum }_{j = 1}^{N} ( {coefficient}_{j} \times ( {variable}_{j} ) \\ P = \frac{exp ( Logit ( P ) )}{1 + exp ( Logit ( P ) )}\end{matrix} \qquad ( iii )$$
wherein:
P is probability that the test subject has aggressive prostate cancer,
the coefficientᵢ is the natural log of the odds ratio of the variable,
the transformed variableᵢ is the natural log of the variableᵢ value,
the coefficientⱼ is the natural log of the odds ratio of the variable,
the variableⱼ is the numerical value of the variableⱼ,
variableⱼ can be one or more of DRE value, Age, or PIRADS score,
if variableⱼ is DRE, a DRE value of 1 indicates an abnormal DRE status and a DRE value of 0 indicates a normal DRE status,
if variableⱼ is PIRADs score, the PIRADS score is 1, 2, 3, 4 or 5, and if a PIRADS score is not available, variableⱼ is 0, and
if variableⱼ is Age, the Age is in years.

11. The method according to any one of claims 1 to 10, wherein said combining of each said analyte level of the series with measurements of said one or more clinical variables obtained from each said subject of the populations maximizes said discrimination.

12. The method according to any one of claims 1 to 11, wherein said combining of each said analyte level of the series with the measurements of one or more clinical variables obtained from each said subject of the populations is conducted in a manner that:
(i) reduces the misclassification rate between the subjects likely to have aggressive CaP and said control subjects; and/or
(ii) increases sensitivity in discriminating between the subjects likely to have aggressive CaP and said control subjects; and/or
(iii) increases specificity in discriminating between the subjects likely to have aggressive CaP and said control subjects.

13. The method according to claim 12, wherein said combining in a manner that reduces the misclassification rate between the subjects likely to have aggressive CaP and said control subjects comprises selecting a suitable true positive and/or true negative rate.

14. The method according to claim 12, wherein said combining in a manner that reduces the misclassification rate between the subjects likely to have aggressive CaP and said control subjects minimizes the misclassification rate.

15. The method according to claim 12, wherein said combining in a manner that reduces the misclassification rate between the subjects likely to have aggressive CaP and said control subjects comprises minimizing the misclassification rate between the subjects likely to have aggressive CaP and said control subjects by identifying a point where the true positive rate intersects the true negative rate.

16. The method according to claim 12, wherein said selecting the threshold value from the combined clinical variable measurement/s and combined analyte level/s in a manner that increases sensitivity in discriminating between the subjects likely to have aggressive CaP and said control subjects increases or maximizes said sensitivity.

17. The method according to claim 12, wherein said selecting the threshold value from the combined clinical variable measurement/s and combined analyte level/s in a manner that increases specificity in discriminating between the subjects likely to have aggressive CaP and said control subjects increases or maximizes said specificity.

18. The method according to any one of claims 1 to 17, wherein the one or more clinical variables and the one or more analytes comprise or consist of:
total PSA, % free PSA, WFDC2 (HE4), Age or
total PSA, % free PSA, WFDC2 (HE4), Age, PV.

19. The method according to any one of claims 1 to 18, wherein the test subject has previously received a positive indication of prostate cancer.

20. The method according to any one of claims 1 to 19, wherein the test subject has previously received a positive indication of prostate cancer by digital rectal exam (DRE) and/or by PSA testing.

21. The method according to any one of claims 1 to 19, wherein the test subject has previously received a positive indication of prostate cancer by MRI PIRADs score.

22. The method according to any one of claims 1 to 19, wherein the test subject has previously received a positive indication of prostate cancer by DRE and/or PSA testing with MRI PIRADs score of 1-3.

23. The method according to any one of claims 1 to 19, wherein the test subject has previously received a positive indication of prostate cancer by DRE and/or PSA testing with MRI PIRADs score of 3.

24. The method according to any one of claims 1 to 23, wherein the series of biological samples obtained from each said population and/or the test subject's biological sample are selected from: whole blood, serum, plasma, saliva, tear/s, urine, tissue, and any combination thereof.

25. The method according to any one of claims 1 to 24, wherein said test subject, said population of subjects likely to have aggressive CaP, and said population of control subjects are human.

26. The method according to any one of claims 1 to 25, further comprising measuring one or more analyte/s in the test subject's biological sample to thereby obtain the analyte level for each said one or more analytes.

27. The method according to claim 26, wherein said measuring of one or more analyte/s in the test subject's biological sample comprises:
(i) measuring one or more fluorescent signals indicative of each said analyte level;
(ii) obtaining a measurement of weight/volume or molarity of said analyte/s in the biological sample;
(iii) measuring an absorbance signal indicative of each said analyte level; or
(iv) using a technique selected from the group consisting of: electrochemiluminescence, mass spectrometry, a protein array technique, high performance liquid chromatography (HPLC), gel electrophoresis, radiolabeling, and any combination thereof.

28. The method according to claim 26 or claim 27, wherein the test subject's biological sample is contacted, or the series of biological samples was contacted, with first and second antibody populations for detection of each said analyte, wherein each said antibody population has binding specificity for one of said analytes, and the first and second antibody populations have different analyte binding specificities.

29. The method according to claim 28, wherein the first and/or second antibody populations are labelled.

30. The method according to claim 29, wherein the first and/or second antibody populations comprise a label selected from the group consisting of a radiolabel, a fluorescent label, a biotin-avidin amplification system, a chemiluminescence system, microspheres, and colloidal gold.

31. The method according to claim 28 or 29, wherein binding of each said antibody population to the analyte is detected by a technique selected from the group consisting of: immunofluorescence, radiolabeling, immunoblotting, Western blotting, enzyme-linked immunosorbent assay (ELISA), flow cytometry, immunoprecipitation, immunohistochemistry, biofilm test, affinity ring test, antibody array, optical density test, and chemiluminescence.

32. The method according to any one of claims 26 to 31, wherein said measuring of each said analyte in the biological sample from the test subject or the series of biological samples obtained from each said population comprises measuring the analytes directly.

33. The method according to any one of claims 26 to 31, wherein said measuring of each said analyte in the biological sample from the test subject or the series of biological samples obtained from each said population comprises detecting a nucleic acid encoding the analytes.

34. The method according to any one of claims 1 to 33, further comprising measuring the two one or more clinical variables in the test subject.

35. The method of any one of claims 1 to 34, further comprising determining said threshold value.

36. The method according to claim 35, wherein determining said threshold value comprises measuring said one or more analyte/s in a series of biological samples obtained from a population of subjects having aggressive CaP and from a population of control subjects not having aggressive CaP, to thereby obtain an analyte level for each said analyte in each said biological sample of the series.

37. The method of any one of claims 1 to 36, further comprising a step of obtaining a biopsy from the test subject to confirm whether the test subject has aggressive CaP.

38. The method of claim 37, further comprising a step of treating a test subject confirmed to have aggressive CaP, optionally wherein the treatment is selected from one or more of surgery (e.g., radical prostatectomy), chemotherapy, radiation therapy (e.g., external beam radiation therapy or brachytherapy (with or without concomitant androgen deprivation therapy)), immunotherapy, hormone therapy, drug treatment and combinations thereof.

39. A method of treating aggressive prostate cancer in a test subject comprising:
(a) having obtained an analyte level for one or more analytes in the test subject's biological sample, and having obtained a measurement of one or more clinical variables from the test subject; and
(b) applying a suitable algorithm and/or transformation to a combination of the clinical variable measurements and analyte level/s of the test subject to thereby generate a test subject score value for comparison to a threshold value; and
(c) determining whether the test subject is likely to have aggressive CaP by comparison of the subject test score value and the threshold value,
wherein:
the one or more analyte/s comprise or consist of WAP four-disulfide core domain protein 2 (WFDC2 (HE4)), and optionally total prostate surface antigen (PSA),
the one or more clinical variables comprise at least one of: % Free PSA, Free PSA, DRE, Prostate Volume (PV), PIRADs score, Age, Family History (FH) and
the threshold value was determined by:
measuring said one or more analyte/s in a series of biological samples obtained from a population of subjects having aggressive CaP and from a population of control subjects not having aggressive CaP, to thereby obtain an analyte level for each said analyte in each said biological sample of the series;
combining each said analyte level of the series with measurements of said one or more clinical variables obtained from each said subject of the populations, in a manner that allows discrimination between aggressive CaP and an absence of aggressive CaP, to thereby generate the threshold value,
wherein the method further comprises:
(i) obtaining a biopsy from the test subject to confirm the presence of aggressive CaP; and
(ii) treating the test subject confirmed to have aggressive CaP, preferably with one or more of surgery (e.g., radical prostatectomy), chemotherapy, radiation therapy (e.g., external beam radiation therapy or brachytherapy (with or without concomitant androgen deprivation therapy)), immunotherapy, hormone therapy, drug treatment or combinations thereof.

40. The method of claim 39, further comprising one or more of the features or steps defined in any one of claims 2 to 36.

41. A kit for use in the method according to any one of claims 1 to 36, comprising reagents and/or components for determining the clinical variable/s and the one or more biomarker/s defined in any one of claims 1 to 36.
